(19) United States
(12) Patent Application Publication
(10) Pub. No.: US 2015/0376590 A1
(43) Pub. Date: Dec. 31, 2015

(54) Thermotolerant Beta-Glucosidase Variants

(71) Applicants: Jon Peter Flash BARTNEK, Naperville, IL (US); Justin Trent STEGE, Naperville, IL (US); BP Corporation North America Inc., Naperville, IL (US)

(72) Inventors: Jon Peter Flash Bartnek, San Diego, CA (US); Justin Trent Stege, San Diego, CA (US)

(73) Assignee: BP Corporation North America Inc., Naperville, IL (US)

(21) Appl. No.: 14/440,273

(22) PCT Filed: Oct. 30, 2013
(86) PCT No.: PCT/US13/67447

§ 371 Date: May 1, 2015

Related U.S. Application Data
(60) Provisional application No. 61/721,799, filed on Nov. 2, 2012.

(51) Int. Cl. C12N 9/42; C12P 7/14

(52) U.S. Cl. CPC C12N 9/2445; C12P 7/14; C12Y 302/01021

(57) ABSTRACT

The present disclosure relates to variant β-glucosidase polypeptides that have enhanced thermostability, and compositions, e.g., cellulase compositions, comprising variant β-glucosidase polypeptides. The variant β-glucosidase polypeptides and related compositions can be used in variety of agricultural and industrial applications. The present disclosure further relates to nucleic acids encoding variant β-glucosidase polypeptides and host cells that recombinantly express the variant β-glucosidase polypeptides.

### REFERENCE TO SEQUENCE LISTING SUBMITTED VIA EFS-WEB

This application is being transmitted by EFS-Web, as authorized and set forth in MPEP §502.05, including a sequence listing submitted under 37 C.F.R. §1.821 in ASCII text file (.txt) format.

### BACKGROUND

Cellulosic biomass is a significant renewable resource for the generation of soluble sugars. These sugars can be used as reactants in various metabolic processes, including fermentation, to produce biofuels, chemical compounds, and other commercially valuable products. For example, fermentation of plant biomass to ethanol is an attractive carbon neutral energy option since the combustion of ethanol from biomass produces no net carbon dioxide in the earth's atmosphere. Further, biomass is readily available, and its fermentation provides an attractive way to dispose of many industrial and agricultural waste products. Finally, plant biomass is a highly renewable resource. Many dedicated energy crops can provide high energy biomass, which may be harvested multiple times each year.

Cellulose is a polymer of the simple sugar glucose covalently bonded by β-1,4-linkages. Cellulose is the most abundant organic compound on earth, making up about 33 percent of all plant matter, about 50 percent of wood, and about 90 percent of products such as cotton. In nature, cellulose is present as part of the lignocellulosic biomass of plants, which is composed of cellulose, hemicellulose, and lignin. The carbohydrate polymers (cellulose and hemicelluloses) are tightly bound to the lignin, by hydrogen and covalent bonds. Cellulose may be pretreated chemically, mechanically, enzymatically or in other ways to increase the susceptibility of cellulose to hydrolysis. Such pretreatment may be followed by the enzymatic conversion of cellulose to cellobiose, cello-oligosaccharides, glucose, and other sugars and sugar polymers, using enzymes that break down the β-1-4 glycosidic bonds of cellulose. These enzymes are collectively referred to as “cellulases.”

Cellulases are divided into three sub-categories of enzymes: 1,4-β-D-glucan glucanohydrolase (“endoglucanase” or “EG”); 1,4-β-D-glucan cellobiohydrolase (“exoglucanase”, “cellobiohydrolase”, or “CBH”); and β-D-glucoside-glucohydrolase (“β-glucosidase”, “beta-glucosidase”, “cellobiase” or “BGL”). Endoglucanases break internal bonds and disrupt the crystalline structure of cellulose, exposing individual cellulose polysaccharide chains (“glucans”). Cellobiohydrolases incrementally shorten the glucan molecules, releasing mainly cellobiose units (a water-soluble β-1,4-linked dimer of glucose) as well as glucose, cellotriose, and cellotetraose. β-Glucosidases split cellobiose into glucose monomers.

Typically, the digestion of cellulose is carried out at high temperatures, at which intermolecular hydrogen bonds are disrupted and recalcitrant cellulose polymers becomes accessible to the cellulase enzymes. Therefore, cellulases used commercially in such processes should be able to withstand high temperatures so as to permit breakdown of cellulose under commercially viable conditions.

Cellulases with improved properties, such as thermal stability, for use in processing cellulosic biomass would reduce costs and increase the efficiency of production of biofuels and other commercially valuable compounds.

### SUMMARY

The present disclosure relates to variant β-glucosidase polypeptides. Most naturally occurring β-glucosidases do not perform well at the high temperatures that are optimal for commercial reasons, for example biomass saccharification reactions that utilize cellulase cocktails. The variant β-glucosidase polypeptides of the disclosure have one or more amino acid substitutions that improve performance at temperatures above 50° C. (e.g., 60° C., 66° C., 70° C., or 80° C.). Such variants are sometimes referred to herein as “thermally tolerant” or “thermotolerant.” In some instances, the variants have an increased specific activity towards a β-glucosidase substrate at ambient temperatures (e.g., 22-25° C.).

Accordingly, the present disclosure provides polypeptides (variant β-glucosidase polypeptides) which have been engineered to incorporate an amino acid substitution that results in increased thermal tolerance, increased specific activity, or both. The variant β-glucosidase polypeptides of the disclosure minimally contain one or more amino acid substitutions selected from Table 1, below, which lists substitution names and corresponding positions in SEQ ID NO:379 and SEQ ID NO:378.

TABLE 1

| | Corresponding position | Corresponding position |
|---|---|---|
| Substitution Name | in SEQ ID NO: 379 | in SEQ ID NO: 378 |
| I63 substitution | I63 | V67 |
| A68 substitution | A68 | E72 |
| A73 substitution | A73 | A77 |
| Y74 substitution | Y74 | Y78 |
| V167 substitution | V167 | C171 |
| V203 substitution | V203 | T207 |
| I216 substitution | I216 | V219 |
| T219 substitution | T219 | T222 |
| K231 substitution | K231 | (none) |
| M246 substitution | M246 | F248 |
| F292 substitution | F292 | I293 |
| S296 substitution | S296 | S297 |
| M325 substitution | M325 | M323 |
| N326 substitution | N326 | N324 |
| E365 substitution | E365 | D363 |
| Y399 substitution | Y399 | F397 |
| V400 substitution | V400 | A398 |
| W401 substitution | W401 | W399 |
| R410 substitution | R410 | A408 |
| D414 substitution | D414 | D412 |
| L427 substitution | L427 | Q425 |
| T441 substitution | T441 | (none) |
| E450 substitution | E450 | A444 |

The one or more amino acid substitutions can be introduced into a β-glucosidase of SEQ ID NO:378, a β-glucosidase of SEQ ID NO:379, or another, preferably bacterial, β-glucosidase. Amino acid positions in other exemplary β-glucosidase polypeptides corresponding to the foregoing amino acid positions in SEQ ID NO:378 and SEQ ID NO:379 are shown in Tables 9A-9C. One, two, three, four, five, six, seven, eight, nine, or ten or more of the amino acid substitutions listed in Table 1 can by introduced into the β-glucosidase. In certain aspects, one or more amino acid substituents are selected from:
(a) an A to G substitution in the amino acid corresponding to amino acid 73 of SEQ ID NO:379 (an “A73G substitution”);
(b) an A to S substitution in the amino acid corresponding to amino acid 73 of SEQ ID NO:379 (an “A73S substitution”);
(c) a Y to L substitution in the amino acid corresponding to amino acid 74 of SEQ ID NO:379 (a “Y74L substitution”);
(d) a V to A substitution in the amino acid corresponding to amino acid 167 of SEQ ID NO:379 (a “V167A substitution”);
(e) a T to A substitution in the amino acid corresponding to amino acid 219 of SEQ ID NO:379 (a “T219A substitution”);
(f) a T to S substitution in the amino acid corresponding to amino acid 219 of SEQ ID NO:379 (a “T219S substitution”);
(g) a K to E substitution in the amino acid corresponding to amino acid 231 of SEQ ID NO:379 (a “K231E substitution”);
(h) a M to H substitution in the amino acid corresponding to amino acid 246 of SEQ ID NO:379 (a “M246H substitution”);
(i) a M to K substitution in the amino acid corresponding to amino acid 246 of SEQ ID NO:379 (a “M246K substitution”);
(j) a F to I substitution in the amino acid corresponding to amino acid 292 of SEQ ID NO:379 (a “F292I substitution”);
(k) a F to V substitution in the amino acid corresponding to amino acid 292 of SEQ ID NO:379 (a “F292V substitution”);
(l) a S to T substitution in the amino acid corresponding to amino acid 296 of SEQ ID NO:379 (a “S296T substitution”);
(m) a M to T substitution in the amino acid corresponding to amino acid 325 of SEQ ID NO:379 (a “M325T substitution”);
(n) a N to G substitution in the amino acid corresponding to amino acid 326 of SEQ ID NO:379 (a “N326G substitution”);
(o) a Y to F substitution in the amino acid corresponding to amino acid 399 of SEQ ID NO:379 (a “Y399F substitution”);
(p) a W to F substitution in the amino acid corresponding to amino acid 401 of SEQ ID NO:379 (a “W401F substitution”);
(q) a T to V substitution in the amino acid corresponding to amino acid 441 of SEQ ID NO:379 (a “T441V substitution”); and
(r) an A to C substitution in the amino acid corresponding to amino acid 449 of SEQ ID NO:379 (a “A449C substitution”).

In certain embodiments, the β-glucosidase variants of the disclosure include one or more, two or more, or three or more of: an M246H substitution, an I216V substitution, and a T219A substitution.

In yet other embodiments, the β-glucosidase variants of the disclosure include a polypeptide comprising a variant β-glucosidase polypeptide as compared to a reference β-glucosidase polypeptide, comprising one or more, two or more, three or more, four or more, five or more, six or more, seven or more, or eight substitutions selected from:
(a) a D7H substitution;
(b) a D154N substitution;
(c) an I216V substitution;
(d) a D243H substitution;
(e) a D302R substitution;
(f) an S317H substitution;
(g) an E365G substitution; and
(h) a V400Y substitution.

In some embodiments, a β-glucosidase variant of the disclosure comprises SEQ ID NO:267.

The β-glucosidase polypeptides of the disclosure generally retain at least 1%, at least 2%, at least 5%, at least 10% and more preferably at least 20% of their specific activity at ambient temperature (22-25° C.) following a 30-minute thermal challenge at 66° C., as compared to wild type β-glucosidase which does not include the same amino acid substitutions. In specific embodiments, the β-glucosidase polypeptides of the disclosure generally retain at least 30%, at least 40%, at least 50%, at least 60%, at least 70% or at least 80% of their specific activity at ambient temperature (22-25° C.) following a 30-minute thermal challenge at 66° C. In certain aspects, the β-glucosidase polypeptides of the disclosure generally retain a percentage of specific activity following a 30-minute thermal challenge at 66° C. that ranges from 1%-50%, 1%-90%, 2%-80%, 2%-40%, 5%-50%, 5%-70%, 10%-90%, 20%-60%, 30%-90%, 30%-80%, or 40%-80% of their activity at ambient temperature (22-25° C.), or a percentage of specific activity in a range bounded by any two of these values (for example 1%-60%, 20%-70%, and so on and so forth).

In certain embodiments, the β-glucosidase polypeptides of the disclosure also retain at least 1%, at least 2%, at least 5%, at least 10% and more preferably at least 20% of their specific activity at ambient temperature (22-25° C.) following a 30-minute thermal challenge at 70° C., 80° C., 84° C. or 86° C. as compared to wild type β-glucosidase which does not include the same amino acid substitutions. In specific embodiments, the β-glucosidase polypeptides of the disclosure generally retain at least 30%, at least 40%, at least 50%, at least 60%, at least 70% or at least 80% of their specific activity at ambient temperature (22-25° C.) following a 30-minute thermal challenge at 70° C., 80° C., 84° C. or 86° C. In certain aspects, the β-glucosidase polypeptides of the disclosure generally retain a percentage of specific activity following a 30-minute thermal challenge at 70° C., 80° C., 84° C. or 86° C. that ranges from 1%-50%, 1%-90%, 2%-80%, 2%-40%, 5%-50%, 5%-70%, 10%-90%, 20%-60%, 30%-90%, 30%-80%, or 40%-80% of their activity at ambient temperature (22-25° C.), or a percentage of specific activity in a range bounded by any two of these values (for example 1%-60%, 20%-70%, and so on and so forth).

The variant β-glucosidase polypeptides of the disclosure typically include an amino acid sequence having at least 40%, at least 45%, at least 48%, at least 50%, at least 60%, at least 70%, at least 80%, at least 90%, at least 95%, or at least 97% sequence identity to the amino acid sequence of SEQ ID NO:378 and/or the amino acid sequence of SEQ ID NO:379. The variant β-glucosidase polypeptides can further include a purification tag, e.g., a histidine tag. Additional embodiments of variant β-glucosidase polypeptides are provided in Section 4.1.

The present disclosure further provides compositions (including cellulase compositions, e.g., whole cellulase compositions, and fermentation broths) comprising variant β-glucosidase polypeptides. Additional embodiments of compositions comprising variant β-glucosidase polypeptides are provided in Section 4.4. The variant β-glucosidase polypeptides and compositions comprising them can be used, inter alia, in processes for saccharifying biomass. Additional details of saccharification reactions, and additional applications of the variant β-glucosidase polypeptides, are provided in Section 4.5.

The present disclosure further provides nucleic acids (e.g., vectors) comprising nucleotide sequences encoding variant β-glucosidase polypeptides as described herein in section 4.2, and recombinant cells engineered to express the variant β-glucosidase polypeptides. The recombinant cell can be a prokaryotic (e.g., bacterial) or eukaryotic (e.g., yeast or filamentous fungal) cell. For recombinant expression in eukaryotic cells, the variant β-glucosidase polypeptides of the disclosure can further include a signal peptide for secretion in the culture media. Further provided are methods of producing and optionally recovering the variant β-glucosidase polypeptides. Additional embodiments of the recombinant expression system suitable for expression and production of the variant β-glucosidase polypeptides are provided in Section 4.3.

### BRIEF DESCRIPTION OF THE FIGURES

FIG. 1 provides a map of a vector used for constructing hexahistidine-tagged β-glucosidase constructs.

FIG. 2 provides data showing the residual activity of wild-type β-glucosidase after 30-minute thermal challenges at the indicated temperatures, serving as the rationale for selecting 66° C. as the temperature for screening the GSSM library.

FIG. 3 provides GSSM thermotolerance screen data for various β-glucosidase variants, with wild-type β-glucosidase activity indicated by the arrows, negative controls marked with an asterisk (*), and a putative thermotolerant β-glucosidase variant highlighted by a circle.

FIG. 4 provides GSSM re-confirmation data, showing results for triplicate assays of β-glucosidase activity for various β-glucosidase variants, with wild-type β-glucosidase activity indicated by the arrows, negative controls marked with an asterisk (*), and putative thermotolerant β-glucosidase variants highlighted by a circle.

TABLE 1 shows amino acid positions that can be substituted in various β-glucosidase variants.

TABLE 2 shows the residual activity remaining following 30-minute thermal challenges at the indicated temperatures for the polypeptides given by SEQ ID NOs:378 and 379.

TABLE 3 shows secondary thermotolerance screen of GSSM mutants.

TABLE 4 shows activity of three thermostable GSSM mutants.

TABLE 5 shows the specific activity after challenge at the indicated temperatures, for the indicated β-glucosidase variants.

TABLE 6A shows the specific activity after challenge at the indicated temperatures, for the indicated β-glucosidase variants of the reassembly library for original parental β-glucosidase.

TABLE 6B shows the specific activity after challenge at the indicated temperatures, for the indicated β-glucosidase variants of the reassembly library for alternate parental β-glucosidase.

TABLE 8 shows substitutions and specific activity after challenge at the indicated temperatures, for the indicated β-glucosidase variants of the reassembly library for original parental β-glucosidase.

TABLE 8 shows substitutions and specific activity after challenge at the indicated temperatures, for the indicated β-glucosidase variants of the reassembly library for alternate parental β-glucosidase.

TABLES 9A-9C show the amino acids in a β-glucosidase of SEQ ID NO:379 that can be substituted to generate thermotolerant β-glucosidase variants, and the corresponding amino acid in other β-glucosidases, including β-glucosidase, of SEQ ID NO:378 and SEQ ID NO:380 (a fusion protein of β-glucosidase SEQ ID NO:379 with a C-terminal histidine tag). Other β-glucosidases are identified by patent publication number and sequence identifier within the patent publication. Thus, “U.S. Pat. No. 8,101,393-0094” refers to SEQ ID NO:94 in U.S. Pat. No. 8,101,393. Table 9A-9C also include β-glucosidases identified by their Protein Data Bank (PDB) and European Molecular Biology Laboratory (EMBL) database accession numbers. All the sequences referred to in Tables 9A-9C are incorporated by reference herein.

### DETAILED DESCRIPTION

The present disclosure relates to variants of a parent β-glucosidase polypeptide, comprising one or more substitutions that result in improved thermal stability and/or specific activity. In various embodiments the variant has improved thermostability compared to the β-glucosidase polypeptide given by SEQ ID NO:378 and/or SEQ ID NO:379. The following subsections describe in greater detail the variant β-glucosidase polypeptides and nucleic acids, as well as exemplary methods of their production, exemplary cellulase compositions comprising them, and some industrial applications of the polypeptides and cellulase compositions.

1.1. Variant β-Glucosidase Polypeptides

β-Glucosidases are cellulase enzymes that split cellobiose into glucose monomers. In some embodiments, the present disclosure provides variants of a parent β-glucosidase polypeptide, comprising one or more substitutions that result in improved thermal stability and/or specific activity. A “parent” β-glucosidase refers to a reference polypeptide sequence, with respect to which one or more amino acid substitutions described herein may be made. A parent β-glucosidase can, but need not be a wild-type β-glucosidase. The term “wild-type” β-glucosidase denotes a β-glucosidase expressed by a naturally occurring microorganism, such as bacterium or yeast found in nature.

In some embodiments, the disclosure provides a polypeptide comprising the amino acid sequence of a variant β-glucosidase, said variant β-glucosidase comprising one or more substitutions as compared to a reference β-glucosidase polypeptide, said one or more substitutions being selected from: a substitution at the amino acid position corresponding to Q3 of SEQ ID NO:379 (a “Q3 substitution”); a substitution at the amino acid position corresponding to K6 of SEQ ID NO:379 (a “K6 substitution”); a substitution at the amino acid position corresponding to D7 of SEQ ID NO:379 (a “D7 substitution”); a substitution at the amino acid position corresponding to T24 of SEQ ID NO:379 (a “T24 substitution”); a substitution at the amino acid position corresponding to V60 of SEQ ID NO:379 (a “V60 substitution”); a substitution at the amino acid position corresponding to I63 of SEQ ID NO:379 (an “I63 substitution”); a substitution at the amino acid position corresponding to A68 of SEQ ID NO:379 (an “A68 substitution”); a substitution at the amino acid position corresponding to A73 of SEQ ID NO:379 (an “A73 substitution”); a substitution at the amino acid position corresponding to Y74 of SEQ ID NO:379 (a “Y74 substitution”); a substitution at the amino acid position corresponding to E105 of SEQ ID NO:379 (an “E105 substitution”); a substitution at the amino acid position corresponding to Q139 of SEQ ID NO:379 (a “Q139 substitution”); a substitution at the amino acid position corresponding to Q142 of SEQ ID NO:379 (a “Q142 substitution”); a substitution at the amino acid position corresponding to D154 of SEQ ID NO:379 (a “D154 substitution”); a substitution at the amino acid position corresponding to V167 of SEQ ID NO:379 (a “V167 substitution”); a substitution at the amino acid position corresponding to N175 of SEQ ID NO:379 (a “N175 substitution”); a substitution at the amino acid position corresponding to V203 of SEQ ID NO:379 (a “V203 substitution”); a substitution at the amino acid position corresponding to I216 of SEQ ID NO:379 (a “I216 substitution”); a substitution at the amino acid position corresponding to T219 of SEQ ID NO:379 (a “T219 substitution”); a substitution at the amino acid position corresponding to K231 of SEQ ID NO:379 (a “K231 substitution”); a substitution at the amino acid position corresponding to D243 of SEQ ID NO:379 (a “D243 substitution”); a substitution at the amino acid position corresponding to M246 of SEQ ID NO:379 (a “M246 substitution”); a substitution at the amino acid position corresponding to F292 of SEQ ID NO:379 (a “F292 substitution”); a substitution at the amino acid position corresponding to S296 of SEQ ID NO:379 (a “S296 substitution”); a substitution at the amino acid position corresponding to T297 of SEQ ID NO:379 (a “T297 substitution”); a substitution at the amino acid position corresponding to D302 of SEQ ID NO:379 (a “D302 substitution”); a substitution at the amino acid position corresponding to H315 of SEQ ID NO:379 (an “H315 substitution”); a substitution at the amino acid position corresponding to S317 of SEQ ID NO:379 (an “S317 substitution”); a substitution at the amino acid position corresponding to M325 of SEQ ID NO:379 (an “M325 substitution”); a substitution at the amino acid position corresponding to N326 of SEQ ID NO:379 (an “N326 substitution”); a substitution at the amino acid position corresponding to N332 of SEQ ID NO:379 (an “N332 substitution”); a substitution at the amino acid position corresponding to E365 of SEQ ID NO:379 (an “E365 substitution”); a substitution at the amino acid position corresponding to Q366 of SEQ ID NO:379 (a “Q366 substitution”); a substitution at the amino acid position corresponding to I378 SEQ ID NO:379 (an “I378 substitution”); a substitution at the amino acid position corresponding to Y399 SEQ ID NO:379 (a “Y399 substitution”); a substitution at the amino acid position corresponding to V400 SEQ ID NO:379 (a “V400 substitution”); a substitution at the amino acid position corresponding to W401 SEQ ID NO:379 (a “W401 substitution”); a substitution at the amino acid position corresponding to S402 SEQ ID NO:379 (an “S402 substitution”); a substitution at the amino acid position corresponding to R410 SEQ ID NO:379 (an “R410 substitution”); a substitution at the amino acid position corresponding to D414 SEQ ID NO:379 (a “D414 substitution”); a substitution at the amino acid position corresponding to K415 SEQ ID NO:379 (a “K415 substitution”); a substitution at the amino acid position corresponding to R416 SEQ ID NO:379 (an “R416 substitution”); a substitution at the amino acid position corresponding to V420 SEQ ID NO:379 (a “V420 substitution”); a substitution at the amino acid position corresponding to L427 SEQ ID NO:379 (an “L427 substitution”); a substitution at the amino acid position corresponding to E428 SEQ ID NO:379 (an “E428 substitution”); a substitution at the amino acid position corresponding to T441 SEQ ID NO:379 (a “T441 substitution”); a substitution at the amino acid position corresponding to L447 SEQ ID NO:379 (an “L447 substitution”); a substitution at the amino acid position corresponding to A449 SEQ ID NO:379 (an “A449 substitution”); a substitution at the amino acid position corresponding to E450 SEQ ID NO:379 (an “E450 substitution”); and a substitution at the amino acid position corresponding to K451 SEQ ID NO:379 (a “K451 substitution”); wherein the one or more substitutions increases thermotolerance as compared to the reference β-glucosidase polypeptide.

In some embodiments, the each of the one or more substitutions is selected from: an A73 substitution selected from A73G and A73S; a Y74 substitution that is Y74L; a V167 substitution that is V167A; a T219 substitution selected from T219A and T219S; a K231 substitution that is K231E; an M246 substitution selected from M246H and M246K; an F292 substitution selected from F292I and F292V; an S296 substitution that is S296T; an M325 substitution that is M325T; an N326 substitution that is N326G; a Y399 substitution that is Y399F; a W401 substitution that is W401F; a T441 substitution that is T441V; and an A449 substitution that is A449C.

The variant β-glucosidase polypeptides of the disclosure can have amino acid substitutions with respect to the reference polypeptide given by SEQ ID NO:379, or with respect to the reference polypeptide given by SEQ ID NO:378, or with respect to any of the reference polypeptides.

With respect to the reference polypeptide given by SEQ ID NO:379, a variant β-glucosidase polypeptide can have one or more, two or more, three or more, four or more, five or more, six or more, seven or more, eight or more, nine or more, ten or more, eleven or more, twelve or more, or thirteen substitutions selected from the group consisting of: D7H, Y74L, D154N, I216V, T219A, T219S, M246H, M246K, F292V, F292I, S296T, Y399F, V400Y, and W401F.

With respect to the reference polypeptide given by SEQ ID NO:379, a variant β-glucosidase polypeptide can have one or more, two or more, three or more, four or more, five or more, six or more, seven or more, eight or more, or nine substitutions selected from the group consisting of D11H, T222A, T222S, D245H, F248H, F248K, I293V, S297T, H303R, R315H, and D363G.

Amino acid positions in other β-glucosidase polypeptides that correspond to substitutions listed herein can be identified through alignment of their sequences with a β-glucosidase of SEQ ID NO:378 or SEQ ID NO:379. Optimal alignment of sequences for comparison can be conducted, e.g., by the local homology algorithm of Smith & Waterman, 1981, Adv. Appl. Math. 2:482-89; by the homology alignment algorithm of Needleman & Wunsch, 1970, J. Mol. Biol. 48:443-53; by the search for similarity method of Pearson & Lipman, 1988, Proc. Nat'l Acad. Sci. USA 85:2444-48, by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

The variant β-glucosidase polypeptides of the disclosure have one or more amino acid substitutions that improve performance at temperatures above 50° C. (e.g., 60° C., 66° C., 70° C., or 80° C.). Such variants are sometimes referred to herein as “thermally tolerant” or “thermotolerant.” In some instances, the variants have an increased specific activity towards a β-glucosidase substrate at ambient temperatures (e.g., 22-25° C.).

In some instances, the variants have increased residual activity following a thermal challenge, compared to a reference β-glucosidase. A thermal challenge involves incubating a variant at a temperature of about 50° C., about 55° C., about 60° C., about 65° C., about 66° C., about 68° C., about 70° C., about 80° C., about 82° C., about 84° C., about 86° C., about 88° C., about 90° C., or greater than 90° C. for a period of time, which can be 10 minutes, 15 minutes, 20 minutes, 25 minutes, 30 minutes, 35 minutes, 40 minutes, 45 minutes, 50 minutes, 60 minutes, or greater than one hour.

a (e.g., a 30-minute thermal challenge at 66° C., a 30-minute thermal challenge at 70° C., or a 30-minute thermal challenge at 80° C.).

The β-glucosidase variants can have improved thermal activity compared to wild-type β-glucosidase. In some embodiments, the thermal activity of the variant β-glucosidase is at least 1.5-fold, preferably at least 2-fold, more preferably at least 5-fold, most preferably at least 7-fold, and most preferably at least 10-fold, and most preferable at least 20-fold more thermally active than the parent enzyme when residual activity is compared following a 30-minute thermal challenge, for example as described in Example(s) 1-3, below.

The property of improved thermal activity can also be referred to as increased thermotolerance or thermal stability.

1.2. Nucleic Acids Encoding Variant β-Glucosidases

The present disclosure further provides nucleic acids (e.g., vectors) comprising nucleotide sequences encoding variant β-glucosidase polypeptides as described herein, and recombinant cells engineered to express the variant β-glucosidase polypeptides.

The disclosure provides isolated, synthetic or recombinant nucleic acids comprising a nucleic acid sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity (homology) to an exemplary nucleic acid of the disclosure, including SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO:15; SEQ ID NO:16; SEQ ID NO:17; SEQ ID NO:18; SEQ ID NO:19; SEQ ID NO:20; SEQ ID NO:21; SEQ ID NO:22; SEQ ID NO:23; SEQ ID NO:24; SEQ ID NO:5; SEQ ID NO:26; SEQ ID NO:27; SEQ ID NO:28; SEQ ID NO:29; SEQ ID NO:30; SEQ ID NO:31; SEQ ID NO:32; SEQ ID NO:33; SEQ ID NO:34; SEQ ID NO:35; SEQ ID NO:36; SEQ ID NO:37; SEQ ID NO:38; SEQ ID NO:39; SEQ ID NO:40; SEQ ID NO:41; SEQ ID NO:42; SEQ ID NO:43; SEQ ID NO:44; SEQ ID NO:45; SEQ ID NO:46; SEQ ID NO:47; SEQ ID NO:48; SEQ ID NO:49; SEQ ID NO:50; SEQ ID NO:51; SEQ ID NO:52; SEQ ID NO:53; SEQ ID NO:54; SEQ ID NO:55; SEQ ID NO:56; SEQ ID NO:57; SEQ ID NO:58; SEQ ID NO:59; SEQ ID NO:60; SEQ ID NO:61; SEQ ID NO:62; SEQ ID NO:63; SEQ ID NO:64; SEQ ID NO:65; SEQ ID NO:66; SEQ ID NO:67; SEQ ID NO:68; SEQ ID NO:69; SEQ ID NO:70; SEQ ID NO:71; SEQ ID NO:72; SEQ ID NO:73; SEQ ID NO:74; SEQ ID NO:75; SEQ ID NO:76; SEQ ID NO:77; SEQ ID NO:78; SEQ ID NO:79; SEQ ID NO:80; SEQ ID NO:81; SEQ ID NO:82; SEQ ID NO:83; SEQ ID NO:84; SEQ ID NO:85; SEQ ID NO:86; SEQ ID NO:87; SEQ ID NO:88; SEQ ID NO:89; SEQ ID NO:90; SEQ ID NO:91; SEQ ID NO:92; SEQ ID NO:93; SEQ ID NO:94; SEQ ID NO:95; SEQ ID NO:96; SEQ ID NO:97; SEQ ID NO:98; SEQ ID NO:99; SEQ ID NO:100; SEQ ID NO:101; SEQ ID NO:102; SEQ ID NO:103; SEQ ID NO:104; SEQ ID NO:105; SEQ ID NO:106; SEQ ID NO:107; SEQ ID NO:108; SEQ ID NO:109; SEQ ID NO:110; SEQ ID NO:111; SEQ ID NO:112; SEQ ID NO:113; SEQ ID NO:114; SEQ ID NO:115; SEQ ID NO:116; SEQ ID NO:117; SEQ ID NO:118; SEQ ID NO:119; SEQ ID NO:120; SEQ ID NO:121; SEQ ID NO:122; SEQ ID NO:123; SEQ ID NO:124; SEQ ID NO:15; SEQ ID NO:126; SEQ ID NO:127; SEQ ID NO:128; SEQ ID NO:129; SEQ ID NO:130; SEQ ID NO:131; SEQ ID NO:132; SEQ ID NO:133; SEQ ID NO:134; SEQ ID NO:135; SEQ ID NO:136; SEQ ID NO:137; SEQ ID NO:138; SEQ ID NO:139; SEQ ID NO:140; SEQ ID NO:141; SEQ ID NO:142; SEQ ID NO:143; SEQ ID NO:144; SEQ ID NO:145; SEQ ID NO:146; SEQ ID NO:147; SEQ ID NO:148; SEQ ID NO:149; SEQ ID NO:150; SEQ ID NO:151; SEQ ID NO:152; SEQ ID NO:153; SEQ ID NO:154; SEQ ID NO:155; SEQ ID NO:156; SEQ ID NO:157; SEQ ID NO:158; SEQ ID NO:159; SEQ ID NO:160; SEQ ID NO:161; SEQ ID NO:162; SEQ ID NO:163; SEQ ID NO:164; SEQ ID NO:165; SEQ ID NO:166; SEQ ID NO:167; SEQ ID NO:168; SEQ ID NO:169; SEQ ID NO:170; SEQ ID NO:171; SEQ ID NO:172; SEQ ID NO:173; SEQ ID NO:174; SEQ ID NO:175; SEQ ID NO:176; SEQ ID NO:177; SEQ ID NO:178; SEQ ID NO:179; SEQ ID NO:180; SEQ ID NO:181; SEQ ID NO:182; SEQ ID NO:183; SEQ ID NO:184; SEQ ID NO:185; SEQ ID NO:186; SEQ ID NO:187; SEQ ID NO:188; SEQ ID NO:189; SEQ ID NO:190; SEQ ID NO:191; SEQ ID NO:192; SEQ ID NO:193; SEQ ID NO:194; SEQ ID NO:195; SEQ ID NO:196; SEQ ID NO:197; SEQ ID NO:198; and/or SEQ ID NO:381; which in alternative embodiments include complementary (partially or completely complementary) (e.g., antisense) sequence, cDNA coding sequences and genomic (e.g., “gDNA”) sequences, and optionally include sequences over a region of at least about 10, 15, 20, 25, 30, 35, 40, 45, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2200, 2250, 2300, 2350, 2400, 2450, 2500, or more residues; or over a region consisting of the protein coding region (e.g., the cDNA) or the genomic sequence; and all of these nucleic acid sequences, and the polypeptides and peptides they encode, encompass “sequences of the disclosure”.

Nucleic acids of the disclosure also include isolated, synthetic or recombinant nucleic acids encoding an exemplary polypeptide (or peptide) of the disclosure which include polypeptides (e.g., enzymes) of the disclosure having the sequence of (or the subsequences of, or enzymatically active fragments of) SEQ ID NO:200; SEQ ID NO:201; SEQ ID NO:202; SEQ ID NO:203; SEQ ID NO:204; SEQ ID NO:205; SEQ ID NO:206; SEQ ID NO:207; SEQ ID NO:208; SEQ ID NO:209; SEQ ID NO:210; SEQ ID NO:211; SEQ ID NO:212; SEQ ID NO:213; SEQ ID NO:214; SEQ ID NO:215; SEQ ID NO:216; SEQ ID NO:217; SEQ ID NO:218; SEQ ID NO:219; SEQ ID NO:220; SEQ ID NO:221; SEQ ID NO:222; SEQ ID NO:223; SEQ ID NO:224; SEQ ID NO:25; SEQ ID NO:226; SEQ ID NO:227; SEQ ID NO:228; SEQ ID NO:229; SEQ ID NO:230; SEQ ID NO:231; SEQ ID NO:232; SEQ ID NO:233; SEQ ID NO:234; SEQ ID NO:235; SEQ ID NO:236; SEQ ID NO:237; SEQ ID NO:238; SEQ ID NO:239; SEQ ID NO:240; SEQ ID NO:241; SEQ ID NO:242; SEQ ID NO:243; SEQ ID NO:244; SEQ ID NO:245; SEQ ID NO:246; SEQ ID NO:247; SEQ ID NO:248; SEQ ID NO:249; SEQ ID NO:250; SEQ ID NO:251; SEQ ID NO:252; SEQ ID NO:253; SEQ ID NO:254; SEQ ID NO:255; SEQ ID NO:256; SEQ ID NO:257; SEQ ID NO:258; SEQ ID NO:259; SEQ ID NO:260; SEQ ID NO:261; SEQ ID NO:262; SEQ ID NO:263; SEQ ID NO:264; SEQ ID NO:265; SEQ ID NO:266; SEQ ID NO:267; SEQ ID NO:268; SEQ ID NO:269; SEQ ID NO:270; SEQ ID NO:271; SEQ ID NO:272; SEQ ID NO:273; SEQ ID NO:274; SEQ ID NO:275; SEQ ID NO:276; SEQ ID NO:277; SEQ ID NO:278; SEQ ID NO:279; SEQ ID NO:280; SEQ ID NO:281; SEQ ID NO:282; SEQ ID NO:283; SEQ ID NO:284; SEQ ID NO:285; SEQ ID NO:286; SEQ ID NO:287; SEQ ID NO:288; SEQ ID NO:289; SEQ ID NO:290; SEQ ID NO:291; SEQ ID NO:292; SEQ ID NO:293; SEQ ID NO:294; SEQ ID NO:295; SEQ ID NO:296; SEQ ID NO:297; SEQ ID NO:298; SEQ ID NO: 299; SEQ ID NO:300; SEQ ID NO:301; SEQ ID NO:302; SEQ ID NO:303; SEQ ID NO:304; SEQ ID NO:305; SEQ ID NO:306; SEQ ID NO:307; SEQ ID NO:308; SEQ ID NO:309; SEQ ID NO:310; SEQ ID NO:311; SEQ ID NO:312; SEQ ID NO:313; SEQ ID NO:314; SEQ ID NO:315; SEQ ID NO:316; SEQ ID NO:317; SEQ ID NO:318; SEQ ID NO:319; SEQ ID NO:320; SEQ ID NO:321; SEQ ID NO:322; SEQ ID NO:323; SEQ ID NO:324; SEQ ID NO:35; SEQ ID NO:326; SEQ ID NO:327; SEQ ID NO:328; SEQ ID NO:329; SEQ ID NO:330; SEQ ID NO:331; SEQ ID NO:332; SEQ ID NO:333; SEQ ID NO:334; SEQ ID NO:335; SEQ ID NO:336; SEQ ID NO:337; SEQ ID NO:338; SEQ ID NO:339; SEQ ID NO:340; SEQ ID NO:341; SEQ ID NO:342; SEQ ID NO:343; SEQ ID NO:344; SEQ ID NO:345; SEQ ID NO:346; SEQ ID NO:347; SEQ ID NO:348; SEQ ID NO:349; SEQ ID NO:350; SEQ ID NO:351; SEQ ID NO:352; SEQ ID NO:353; SEQ ID NO:354; SEQ ID NO:355; SEQ ID NO:356; SEQ ID NO:357; SEQ ID NO:358; SEQ ID NO:359; SEQ ID NO:360; SEQ ID NO:361; SEQ ID NO:362; SEQ ID NO:363; SEQ ID NO:364; SEQ ID NO:365; SEQ ID NO:366; SEQ ID NO:367; SEQ ID NO:368; SEQ ID NO:369; SEQ ID NO:370; SEQ ID NO:371; SEQ ID NO:372; SEQ ID NO:373; SEQ ID NO:374; SEQ ID NO:375; SEQ ID NO:376; SEQ ID NO:377; SEQ ID NO:378; SEQ ID NO:379; and/or SEQ ID NO:380;

1.3. Recombinant Expression of Variant β-Glucosidase Polypeptides

1.3.1. Cell Culture Systems

The disclosure also provides recombinant cells engineered to express variant β-glucosidase polypeptides. Suitably, the variant β-glucosidase polypeptide is encoded by a nucleic acid operably linked to a promoter.

Where recombinant expression in a filamentous fungal host is desired, the promoter can be a filamentous fungal promoter. The nucleic acids can be, for example, under the control of heterologous promoters. The variant β-glucosidase polypeptides can also be expressed under the control of constitutive or inducible promoters. Examples of promoters that can be used include, but are not limited to, a cellulase promoter, a xylanase promoter, the 1818 promoter (previously identified as a highly expressed protein by EST mapping Trichoderma), and a viral promoter. For example, the promoter can suitably be a cellobiohydrolase, endoglucanase, or β-glucosidase promoter. A particularly suitable promoter can be, for example, a T. reesei cellobiohydrolase, endoglucanase, or β-glucosidase promoter. Non-limiting examples of promoters include a cbh1, cbh2, egl1, egl2, egl3, egl4, egl5, pki1, gpd1, xyn1, or xyn2 promoter.

Suitable host cells include cells of any microorganism (e.g., cells of a bacterium, a protist, an alga, a fungus (e.g., a yeast or filamentous fungus), or other microbe), and are preferably cells of a bacterium, a yeast, or a filamentous fungus.

When expressing in a eukaryotic host cell, a recombinant variant β-glucosidase polypeptide could be fused to a signal peptide (also known as a signal sequence) in order to promote secretion. Signal peptides and methods of attaching them to recombinant polypeptides are known in the art.

Suitable host cells of the bacterial genera include, but are not limited to, cells of Escherichia, Bacillus, Lactobacillus, Pseudomonas, and Streptomyces. Suitable cells of bacterial species include, but are not limited to, cells of Escherichia coli, Bacillus subtilis, Bacillus licheniformis, Lactobacillus brevis, Pseudomonas aeruginosa, and Streptomyces lividans.

Suitable host cells of the genera of yeast include, but are not limited to, cells of Saccharomyces, Schizosaccharomyces, Candida, Hansenula, Pichia, Kluyveromyces, and Phaffia. Suitable cells of yeast species include, but are not limited to, cells of Saccharomyces cerevisiae, Schizosaccharomyces pombe, Candida albicans, Hansenula polymorpha, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaetomium, Cryptococcus, Filobasidium, Fusarium, Gibberella, Humicola, Hypocrea, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma. More preferably, the recombinant cell is a Trichoderma sp. (e.g., Trichoderma reesei), Penicillium sp., Humicola sp. (e.g., Humicola insolens); Aspergillus sp. (e.g., Aspergillus niger), Chrysosporium sp., Fusarium sp., or Hypocrea sp. Suitable cells can also include cells of various anamorph and teleomorph forms of these filamentous fungal genera.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum, Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

The engineered host cells can be cultured in conventional nutrient media modified as appropriate for activating promoters, selecting transformants, or amplifying the nucleic acid sequence encoding the variant β-glucosidase polypeptide. Culture conditions, such as temperature, pH and the like, are those previously used with the host cell selected for expression, and will be apparent to those skilled in the art. As noted, many references are available for the culture and production of many cells, including cells of bacterial and fungal origin. Cell culture media in general are set forth in Atlas and Parks (eds.), 1993, The Handbook of Microbiological Media, CRC Press, Boca Raton, Fla., which is incorporated herein by reference. For recombinant expression in filamentous fungal cells, the cells are cultured in a standard medium containing physiological salts and nutrients, such as described in Pourquie et al., 1988, Biochemistry and Genetics of Cellulose Degradation, eds. Aubert, et al., Academic Press, pp. 71-86; and Ilmen et al., 1997, Appl. Environ. Microbiol. 63:1298-1306. Culture conditions are also standard, e.g., cultures are incubated at 28° C. in shaker cultures or fermenters until desired levels of variant β-glucosidase expression are achieved. Preferred culture conditions for a given filamentous fungus may be found in the scientific literature and/or from the source of the fungi such as the American Type Culture Collection (ATCC). After fungal growth has been established, the cells are exposed to conditions effective to cause or permit the expression of a variant β-glucosidase.

In cases where a variant β-glucosidase coding sequence is under the control of an inducible promoter, the inducing agent, e.g., a sugar, metal salt or antibiotics, is added to the medium at a concentration effective to induce variant β-glucosidase expression.

In one embodiment, the recombinant cell is an Aspergillus niger, which is a useful strain for obtaining overexpressed polypeptide. For example A. niger var. awamori dgr246 is known to product elevated amounts of secreted cellulases (Goedegebuur et al., 2002, Curr. Genet. 41:89-98). Other strains of Aspergillus niger var awamori such as GCDAP3, GCDAP4 and GAP3-4 are known (Ward et al., 1993, Appl. Microbiol. Biotechnol. 39:738-743).

In another embodiment, the recombinant cell is a Trichoderma reesei, which is a useful strain for obtaining overexpressed polypeptide. For example, RL-P37, described by Sheir-Neiss et al., 1984, Appl. Microbiol. Biotechnol. 20:46-53, is known to secrete elevated amounts of cellulase enzymes. Functional equivalents of RL-P37 include Trichoderma reesei strain RUT-C30 (ATCC No. 56765) and strain QM9414 (ATCC No. 26921). It is contemplated that these strains would also be useful in overexpressing variant β-glucosidase polypeptides.

Cells expressing the variant β-glucosidase polypeptides of the disclosure can be grown under batch, fed-batch or continuous fermentations conditions. Classical batch fermentation is a closed system, wherein the compositions of the medium is set at the beginning of the fermentation and is not subject to artificial alternations during the fermentation. A variation of the batch system is a fed-batch fermentation in which the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

1.3.2. Recombinant Expression in Plants

The disclosure provides transgenic plants and seeds that recombinantly express a variant β-glucosidase polypeptide. The disclosure also provides plant products, e.g., oils, seeds, leaves, extracts and the like, comprising a variant β-glucosidase polypeptide.

The transgenic plant can be dicotyledonous (a dicot) or monocotyledonous (a monocot). The disclosure also provides methods of making and using these transgenic plants and seeds. The transgenic plant or plant cell expressing a variant β-glucosidase can be constructed in accordance with any method known in the art. See, for example, U.S. Pat. No. 6,309,872. T. reesei β-glucosidase has been successfully expressed in transgenic tobacco (Nicotiana tabaccum) and potato (Solanum tuberosum). See Hooker et al., 2000, in Glycosyl Hydrolases for Biomass Conversion, ACS Symposium Series, Vol. 769, Chapter 4, pp. 55-90.

In a particular aspect, the present disclosure provides for the expression of β-glucosidase variants in transgenic plants or plant organs and methods for the production thereof. DNA expression constructs are provided for the transformation of plants with a nucleic acid encoding the variant β-glucosidase polypeptide, preferably under the control of regulatory sequences which are capable of directing expression of the variant β-glucosidase polypeptide. These regulatory sequences include sequences capable of directing transcription in plants, either constitutively, or in stage and/or tissue specific manners.

The expression of variant β-glucosidase polypeptides in plants can be achieved by a variety of means. Specifically, for example, technologies are available for transforming a large number of plant species, including dicotyledonous species (e.g., tobacco, potato, tomato, Petunia, Brassica) and monocot species. Additionally, for example, strategies for the expression of foreign genes in plants are available. Additionally still, regulatory sequences from plant genes have been identified that are serviceable for the construction of chimeric genes that can be functionally expressed in plants and in plant cells (e.g., Klee, 1987, Ann. Rev. of Plant Phys. 38:467-486; Clark et al., 1990, Virology 179(2):640-7; Smith et al., 1990, Mol. Gen. Genet. 224(3):477-81.

The introduction of nucleic acids into plants can be achieved using several technologies including transformation with Agrobacterium tumefaciens or Agrobacterium rhizogenes. Non-limiting examples of plant tissues that can be transformed include protoplasts, microspores or pollen, and explants such as leaves, stems, roots, hypocotyls, and cotyls. Furthermore, DNA encoding a variant β-glucosidase can be introduced directly into protoplasts and plant cells or tissues by microinjection, electroporation, particle bombardment, and direct DNA uptake.

Variant β-glucosidase polypeptides can be produced in plants by a variety of expression systems. For instance, the use of a constitutive promoter such as the 35S promoter of Cauliflower Mosaic Virus (Guilley et al., 1982, Cell 30:763-73) is serviceable for the accumulation of the expressed protein in virtually all organs of the transgenic plant. Alternatively, promoters that are tissue-specific and/or stage-specific can be used (Higgins, 1984, Annu. Rev. Plant Physiol. 35:191-221; Shotwell and Larkins, 1989, In: The Biochemistry of Plants Vol. 15 (Academic Press, San Diego: Stumpf and Conn, eds.), p. 297), permit expression of variant β-glucosidase polypeptides in a target tissue and/or during a desired stage of development.

A recombinant cell of the disclosure can be engineered to express, in addition to a β-glucosidase polypeptide of the disclosure, one or more cellulase and/or other proteins useful in a cellulotyic reaction, for example a hemicellulase or an accessory polypeptide, optionally in secreted form. Cellulases are known in the art as enzymes that hydrolyze cellulose (β-1,4-glucan or β-D-glucosidic linkages) resulting in the formation of glucose, cellobiose, cellooligosaccharides, and the like. Hemicellulases are enzymes that hydrolyze hemicellulose (a branched polymer of D-xylose linked by β-1,4-glucosyl linkages, arabinose and other attached sugars) and other substrates to their constituent sugars. Accessory polypeptides are present in cellulase preparations that aid in the enzymatic digestion of cellulose. Thus, such recombinant cells can be advantageously used to produce cellulase compositions, as described in 4.4 below.

Cellulase enzymes have been traditionally divided into three major classes: endoglucanases (“EG”), exoglucanases or cellobiohydrolases (“CBH”) and β-glucosidases (“BG”) (Knowles et al., 1987, TIBTECH 5:255-261; Schulein, 1988, Methods in Enzymology 160(25):234-243).

Endoglucanases:

Endoglucanases break internal bonds and disrupt the crystalline structure of cellulose, exposing individual cellulose polysaccharide chains (“glucans”). Endoglucanases include polypeptides classified as Enzyme Commission no. (“EC”) 3.2.1.4) or which are capable of catalyzing the endohydrolysis of 1,4-β-D-glucosidic linkages in cellulose, lichenin or cereal β-D-glucans. Enzyme Commission numbering is a numerical classification scheme for enzymes.

Examples of suitable bacterial endoglucanases include, but are not limited to, Acidothermus cellulolyticus endoglucanase (WO 91/05039; WO 93/15186; U.S. Pat. No. 5,275,944; WO 96/02551; U.S. Pat. No. 5,536,655, WO 00/70031, WO 05/093050); Thermobifida fusca endoglucanase III (WO 05/093050); and Thermobifida fusca endoglucanase V (WO 05/093050).

Examples of suitable fungal endoglucanases include, but are not limited to, Trichoderma reesei endoglucanase I (Penttila et al., 1986, Gene 45: 253-263; GenBank accession no. M15665); Trichoderma reesei endoglucanase II (Saloheimo et al., 1988, Gene 63:11-22; GenBank accession no. M19373); Trichoderma reesei endoglucanase III (Okada et al., 1988, Appl. Environ. Microbiol. 64: 555-563; GenBank accession no. AB003694); Trichoderma reesei endoglucanase IV (Saloheimo et al., 1997, Eur. J. Biochem. 249: 584-591; GenBank accession no. Y11113); and Trichoderma reesei endoglucanase V (Saloheimo et al., 1994, Molecular Microbiology 13: 219-228; GenBank accession no. Z33381); Aspergillus aculeatus endoglucanase (Ooi et al., 1990, Nucleic Acids Research 18: 5884); Aspergillus kawachii endoglucanase (Sakamoto et al., 1995, Current Genetics 27: 435-439); Chrysosporium sp. C1 endoglucanase (U.S. Pat. No. 6,573,086; GenPept accession no. AAQ38150); Corynascus heterothallicus endoglucanase (U.S. Pat. No. 6,855,531; GenPept accession no. AAY00844); Erwinia carotovara endoglucanase (Saarilahti et al., 1990, Gene 90: 9-14); Fusarium oxysporum endoglucanase (GenBank accession no. L29381); Humicola grisea var. thermoidea endoglucanase (GenBank accession no. AB003107); Melanocarpus albomyces endoglucanase (GenBank accession no. MAL515703); Neurospora crassa endoglucanase (GenBank accession no. XM.sub.—324477); Piromyces equi endoglucanase (Eberhardt et al., 2000, Microbiology 146: 1999-2008; GenPept accession no. CAB92325); Rhizopus oryzae endoglucanase (Moriya et al., 2003, J. Bacteriology 185: 1749-1756; GenBank accession nos. AB047927, AB056667, and AB056668); and Thielavia terrestris endoglucanase (WO 2004/053039; EMBL accession no. CQ827970).

Cellobiohydrolases:

Cellobiohydrolases incrementally shorten the glucan molecules, releasing mainly cellobiose units (a water-soluble β-1,4-linked dimer of glucose) as well as glucose, cellotriose, and cellotetraose. Cellobiohydrolases include polypeptides classified as EC 3.2.1.91 or which are capable of catalyzing the hydrolysis of 1,4-β-D-glucosidic linkages in cellulose or cellotetraose, releasing cellobiose from the ends of the chains. Exemplary cellobiohydrolases include Trichoderma reesei cellobiohydrolase I (CEL7A) (Shoemaker et al., 1983, Biotechnology (N.Y.) 1: 691-696); Trichoderma reesei cellobiohydrolase II (CEL6A) (Teen et al., 1987, Gene 51: 43-52); Chrysosporium lucknowense CEL7 cellobiohydrolase (WO 2001/79507); Myceliophthora thermophila CEL7 (WO 2003/000941); and Thielavia terrestris cellobiohydrolase (WO 2006/074435).

β-Glucosidases:

β-Glucosidases split cellobiose into glucose monomers. β-glucosidases include polypeptides classified as EC 3.2.1.21 or which are capable of catalyzing the hydrolysis of terminal, non-reducing β-D-glucose residues with release of β-D-glucose. Exemplary β-glucosidases can be obtained from Cochliobolus heterostrophus (SEQ ID NO:34), Aspergillus oryzae (WO 2002/095014), Aspergillus fumigatus (WO 2005/047499), Penicillium brasilianum (e.g., Penicillium brasilianum strain IBT 20888) (WO 2007/019442), Aspergillus niger (Dan et al., 2000, J. Biol. Chem. 275: 4973-4980), Aspergillus aculeatus (Kawaguchi et al., 1996, Gene 173: 287-288), Penicillium funiculosum (WO 2004/078919), S. pombe (Wood et al., 2002, Nature 415: 871-880), T. reesei (e.g., β-glucosidase 1 (U.S. Pat. No. 6,022,725), β-glucosidase 3 (U.S. Pat. No. 6,982,159), β-glucosidase 4 (U.S. Pat. No. 7,045,332), β-glucosidase 5 (U.S. Pat. No. 7,005,289), β-glucosidase 6 (U.S. Publication No. 20060258554), or β-glucosidase 7 (U.S. Publication No. 20060258554)).

Hemicellulases:

The recombinantly expressed hemicellulase can be any class of hemicellulase, including an endoxylanase, a β-xylosidase, an α-L-arabionofuranosidase, an α-D-glucuronidase, an acetyl xylan esterase, a feruloyl esterase, a coumaroyl esterase, an α-galactosidase, a α-galactosidase, a β-mannanase or a β-mannosidase.

Endoxylanases include any polypeptide classified EC 3.2.1.8 or which is capable of catalyzing the endohydrolysis of 1,4-β-D-xylosidic linkages in xylans. Endoxylanases also include polypeptides classified as EC 3.2.1.136 or which are capable of hydrolyzing 1,4 xylosidic linkages in glucuronoarabinoxylans.

β-xylosidases include any polypeptide classified as EC 3.2.1.37 or which is capable of catalyzing the hydrolysis of 1,4-β-D-xylans to remove successive D-xylose residues from the non-reducing termini. β-xylosidases may also hydrolyze xylobiose.

α-L-arabinofuranosidases include any polypeptide classified as EC 3.2.1.55 or which is capable of acting on α-L-arabinofuranosides, α-L-arabinans containing (1,2) and/or (1,3)- and/or (1,5)-linkages, arabinoxylans or arabinogalactans.

α-D-glucuronidases include any polypeptide classified as EC 3.2.1.139 or which is capable of catalyzing a reaction of the following form: α-D-glucuronoside+H(2)O=an alcohol+D-glucuronate. α-D-glucuronidases may also hydrolyse 4-O-methylated glucoronic acid, which can also be present as a substituent in xylans. α-D-glucuronidases also include polypeptides classified as EC 3.2.1.131 or which are capable of catalying the hydrolysis of α-1,2-(4-O-methyl)glucuronosyl links.

Acetyl xylan esterases include any polypeptide classified as EC 3.1.1.72 or which is capable of catalyzing the deacetylation of xylans and xylo-oligosaccharides. Acetyl xylan esterases may catalyze the hydrolysis of acetyl groups from polymeric xylan, acetylated xylose, acetylated glucose, α-napthyl acetate or p-nitrophenyl acetate but, typically, not from triacetylglycerol. Acetyl xylan esterases typically do not act on acetylated mannan or pectin.

Feruloyl esterases include any polypeptide classified as EC 3.1.1.73 or which is capable of catalyzing a reaction of the form: feruloyl-saccharide+H(2)O=ferulate+saccharide. The saccharide may be, for example, an oligosaccharide or a polysaccharide. A feruloyl esterase may catalyze the hydrolysis of the 4-hydroxy-3-methoxycinnamoyl (feruloyl) group from an esterified sugar, which is usually arabinose in natural substrates, while p-nitrophenol acetate and methyl ferulate are typically poorer substrates. Feruloyl esterase are sometimes considered hemicellulase accessory enzymes, since they may help xylanases and pectinases to break down plant cell wall hemicellulose and pectin.

Coumaroyl esterases include any polypeptide classified as EC 3.1.1.73 or which is capable of catalyzing a reaction of the form: coumaroyl-saccharide+H(2)O=coumarate+saccharide. The saccharide may be, for example, an oligosaccharide or a polysaccharide. Because some coumaroyl esterases are classified as EC 3.1.1.73 they may also be referred to as feruloyl esterases.

α-galactosidases include any polypeptide classified as EC 3.2.1.22 or which is capable of catalyzing the hydrolysis of of terminal, non-reducing α-D-galactose residues in α-D-galactosides, including galactose oligosaccharides, galactomannans, galactans and arabinogalactans. α-galactosidases may also be capable of hydrolyzing α-D-fucosides.

β-galactosidases include any polypeptide classified as EC 3.2.1.23 or which is capable of catalyzing the hydrolysis of terminal non-reducing β-D-galactose residues in β-D-galactosides. β-galactosidases may also be capable of hydrolyzing α-L-arabinosides.

β-mannanases include any polypeptide classified as EC 3.2.1.78 or which is capable of catalyzing the random hydrolysis of 1,4-β-D-mannosidic linkages in mannans, galactomannans and glucomannans.

β-mannosidases include any polypeptide classified as EC 3.2.1.25 or which is capable of catalyzing the hydrolysis of terminal, non-reducing β-D-mannose residues in β-D-mannosides.

Suitable hemicellulases include T. reesei α-arabinofuranosidase I (ABF1), α-arabinofuranosidase I1 (ABF2), α-arabinofuranosidase III (ABF3), α-galactosidase I (AGL1), α-galactosidase I1 (AGL2), α-galactosidase III (AGL3), acetyl xylan esterase I (AXE1), acetyl xylan esterase III (AXE3), endoglucanase V1 (EG6), endoglucanase VIII (EG8), α-glucuronidase I (GLR1), β-mannanase (MAN1), polygalacturonase (PEC2), xylanase I (XYN1), xylanase I1 (XYN2), xylanase III (XYN3), and β-xylosidase (BXL1).

Accessory Polypeptides:

Accessory polypeptides are present in cellulase preparations that aid in the enzymatic digestion of cellulose (see, e.g., WO 2009/026722 and Harris et al., 2010, Biochemistry, 49:3305-3316). In some embodiments, the accessory polypeptide is an expansin or swollenin-like protein. Expansins are implicated in loosening of the cell wall structure during plant cell growth (see, e.g., Salheimo et al., 2002, Eur. J. Biochem., 269:4202-4211). Expansins have been proposed to disrupt hydrogen bonding between cellulose and other cell wall polysaccharides without having hydrolytic activity. In this way, they are thought to allow the sliding of cellulose fibers and enlargement of the cell wall. Swollenin, an expansin-like protein, contains an N-terminal Carbohydrate Binding Module Family 1 domain (CBD) and a C-terminal expansin-like domain. In some embodiments, an expansin-like protein and/or swollenin-like protein comprises one or both of such domains and/or disrupts the structure of cell walls (e.g., disrupting cellulose structure), optionally without producing detectable amounts of reducing sugars. Other types of accessory proteins include cellulose integrating proteins, scaffoldins and/or a scaffoldin-like proteins (e.g., CipA or CipC from Clostridium thermocellum or Clostridium cellulolyticum respectively). Other exemplary accessory proteins are cellulose induced proteins and/or modulating proteins (e.g., as encoded by cip1 or cip2 gene and/or similar genes from Trichoderma reesei; see e.g., Foreman et al., 2003, J. Biol. Chem., 278:31988-31997.

1.4. Compositions of Variant β-Glucosidase Polypeptides

In general, a variant β-glucosidase polypeptide produced in cell culture is secreted into the medium and may be purified or isolated, e.g., by removing unwanted components from the cell culture medium. However, in some cases, a variant β-glucosidase polypeptide may be produced in a cellular form necessitating recovery from a cell lysate. In such cases the variant β-glucosidase polypeptide is purified from the cells in which it was produced using techniques routinely employed by those of skill in the art. Examples include, but are not limited to, affinity chromatography (Van Tilbeurgh et al., 1984, FEBS Lett. 169(2):215-218), ion-exchange chromatographic methods (Goyal et al., 1991, Bioresource Technology, 36:37-50; Fliess et al., 1983, Eur. J. Appl. Microbiol. Biotechnol. 17:314-318; Bhikhabhai et al., 1984, J. Appl. Biochem. 6:336-345; Ellouz et al., 1987, Journal of Chromatography, 396:307-317), including ion-exchange using materials with high resolution power (Medve et al., 1998, J. Chromatography A, 808:153-165), hydrophobic interaction chromatography (Tomaz and Queiroz, 1999, J. Chromatography A, 865:123-128), and two-phase partitioning (Brumbauer et al., 1999, Bioseparation 7:287-295).

The variant β-glucosidase polypeptides of the disclosure are suitably used in cellulase compositions. Cellulases are known in the art as enzymes that hydrolyze cellulose (beta-1,4-glucan or beta D-glucosidic linkages) resulting in the formation of glucose, cellobiose, cellooligosaccharides, and the like. Cellulase enzymes have been traditionally divided into three major classes: endoglucanases (“EG”), exoglucanases or cellobiohydrolases (EC 3.2.1.91) (“CBH”) and β-glucosidases (EC 3.2.1.21) (“BG”) (Knowles et al., 1987, TIBTECH 5:255-261; Schulein, 1988, Methods in Enzymology 160(25):234-243).

Certain fungi produce complete cellulase systems which include exo-cellobiohydrolases or CBH-type cellulases, endoglucanases or EG-type cellulases and β-glucosidases or BG-type cellulases (Schulein, 1988, Methods in Enzymology 160(25):234-243). Such cellulase compositions are referred to herein as “whole” cellulases.

The cellulase compositions of the disclosure typically include, in addition to a variant β-glucosidase polypeptide, one or more cellobiohydrolases and/or endoglucanases and, optionally, one or more β-glucosidases other than the variant β-glucosidase polypeptides of the disclosure can be included. In their crudest form, cellulase compositions contain the microorganism culture that produced the enzyme components. “Cellulase compositions” also refers to a crude fermentation product of the microorganisms. A crude fermentation is preferably a fermentation broth that has been separated from the microorganism cells and/or cellular debris (e.g., by centrifugation and/or filtration). In some cases, the enzymes in the broth can be optionally diluted, concentrated, partially purified or purified and/or dried. The variant β-glucosidase polypeptide can be co-expressed with one or more of the other components of the cellulase composition or it can be expressed separately, optionally purified and combined with a composition comprising one or more of the other cellulase components.

When employed in cellulase compositions, the variant β-glucosidase is generally present in an amount sufficient to allow release of soluble sugars from the biomass. The amount of variant β-glucosidase enzymes added depends upon the type of biomass to be saccharified which can be readily determined by the skilled artisan. In certain embodiments, the weight percent of variant β-glucosidase polypeptide is suitably at least 1, at least 5, at least 10, or at least 20 weight percent of the total polypeptides in a cellulase composition. Exemplary cellulase compositions include a variant β-glucosidase of the disclosure in an amount ranging from about 1 to about 5 weight percent, from about 1 to about 10 weight percent, from about 1 to about 15 weight percent, from about 1 to about 20 weight percent, from about 1 to about 25 weight percent, from about 5 to about 10 weight percent, from about 5 to about 15 weight percent, from about 5 to about 20 weight percent, from about 5 to about 25 weight percent, from about 5 to about 30 weight percent, from about 5 to about 35 weight percent, from about 5 to about 40 weight percent, from about 5 to about 45 weight percent, from about 5 to about 50 weight percent, from about 10 to about 20 weight percent, from about 10 to about 25 weight percent, from about 10 to about 30 weight percent, from about 10 to about 35 weight percent, from about 10 to about 40 weight percent, from about 10 to about 45 weight percent, from about 10 to about 50 weight percent, from about 15 to about 20 weight percent, from about 15 to about 25 weight percent, from about 15 to about 30 weight percent, or from about 15 to about 35 weight percent of the total polypeptides in the composition.

1.5. Utility of Variant β-Glucosidase Polypeptides

It can be appreciated that the variant β-glucosidase polypeptides of the disclosure and compositions comprising the variant β-glucosidase polypeptides find utility in a wide variety of applications, for example in detergent compositions that exhibit enhanced cleaning ability, function as a softening agent and/or improve the feel of cotton fabrics (e.g., “stone washing” or “biopolishing”), or in cellulase compositions for degrading wood pulp into sugars (e.g., for biofuel production). Other applications include the treatment of mechanical pulp (Pere et al., 1996, Tappi Pulping Conference, pp. 693-696 (Nashville, Tenn., Oct. 27-31, 1996)), for use as a feed additive (see, e.g., WO 91/04673) and in grain wet milling.

1.5.1. Saccharification Reactions

Biofuels such as ethanol can be produced via saccharification and fermentation processes from cellulosic biomass such as trees, herbaceous plants, municipal solid waste and agricultural and forestry residues. However, the ratio of individual cellulase enzymes within a naturally occurring cellulase mixture produced by a microbe may not be the most efficient for rapid conversion of cellulose in biomass to glucose. The use of optimized β-glucosidase activity may greatly enhance the production of ethanol.

Cellulase compositions comprising one or more of the variant β-glucosidase polypeptides of the disclosure can be used in saccharification reaction to produce simple sugars for fermentation. Accordingly, the present disclosure provides methods for saccharification comprising contacting biomass with a cellulase composition comprising a variant β-glucosidase polypeptide of the disclosure and, optionally, subjecting the resulting sugars to fermentation by a microorganism.

The term “biomass,” as used herein, refers to any composition comprising cellulose (optionally also hemicellulose and/or lignin). As used herein, biomass includes, without limitation, seeds, grains, tubers, plant waste or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), wood (including, e.g., wood chips, processing waste), paper, pulp, and recycled paper (including, e.g., newspaper, printer paper, and the like). Other biomass materials include, without limitation, potatoes, soybean (e.g., rapeseed), barley, rye, oats, wheat, beets, and sugar cane bagasse.

The saccharified biomass (e.g., lignocellulosic material processed by enzymes of the disclosure) can be made into a number of bio-based products, via processes such as, e.g., microbial fermentation and/or chemical synthesis. As used herein, “microbial fermentation” refers to a process of growing and harvesting fermenting microorganisms under suitable conditions. The fermenting microorganism can be any microorganism suitable for use in a desired fermentation process for the production of bio-based products. Suitable fermenting microorganisms include, without limitation, filamentous fungi, yeast, and bacteria. The saccharified biomass can, for example, be made it into a fuel (e.g., a biofuel such as a bioethanol, biobutanol, biomethanol, a biopropanol, a biodiesel, a jet fuel, or the like) via fermentation and/or chemical synthesis. The saccharified biomass can, for example, also be made into a commodity chemical (e.g., ascorbic acid, isoprene, 1,3-propanediol), lipids, amino acids, polypeptides, and enzymes, via fermentation and/or chemical synthesis.

Thus, in certain aspects, the variant β-glucosidase polypeptides of the disclosure find utility in the generation of ethanol from biomass in either separate or simultaneous saccharification and fermentation processes. Separate saccharification and fermentation is a process whereby cellulose present in biomass is saccharified into simple sugars (e.g., glucose) and the simple sugars subsequently fermented by microorganisms (e.g., yeast) into ethanol. Simultaneous saccharification and fermentation is a process whereby cellulose present in biomass is saccharified into simple sugars (e.g., glucose) and, at the same time and in the same reactor, microorganisms (e.g., yeast) ferment the simple sugars into ethanol.

Prior to saccharification, biomass is preferably subject to one or more pretreatment step(s) in order to render cellulose material more accessible or susceptible to enzymes and thus more amenable to hydrolysis by the variant β-glucosidase polypeptides of the disclosure.

1.5.2. Detergent Compositions

The present disclosure also provides detergent compositions comprising a variant β-glucosidase polypeptide of the disclosure. The detergent compositions may employ besides the variant β-glucosidase polypeptide one or more of a surfactant, including anionic, non-ionic and ampholytic surfactants; a hydrolase; a bleaching agents; a bluing agent; a caking inhibitors; a solubilizer; and a cationic surfactant. All of these components are known in the detergent art.

The variant β-glucosidase polypeptide is preferably provided as part of cellulase composition. The cellulase composition can be employed from about 0.00005 weight percent to about 5 weight percent or from about 0.0002 weight percent to about 2 weight percent of the total detergent composition. The cellulase composition can be in the form of a liquid diluent, granule, emulsion, gel, paste, and the like. Such forms are known to the skilled artisan. When a solid detergent composition is employed, the cellulase composition is preferably formulated as granules.

### EXAMPLES

### Example 1

### Selecting a Lead β-Glucosidase for Thermotolerance Evolution

Identification of a lead β-glucosidase enzyme for evolution for thermotolerance was carried out utilizing an internal collection of β-glucosidase enzymes identified by either sequence homology to known β-glucosidases, or by the demonstration of activity on cellobiose or other substrates using known methods. The two β-glucosidase enzymes that were selected for further screening are given by SEQ ID NO:378 and SEQ ID NO:379.

### Expression of β-Glucosidase Candidate Enzymes

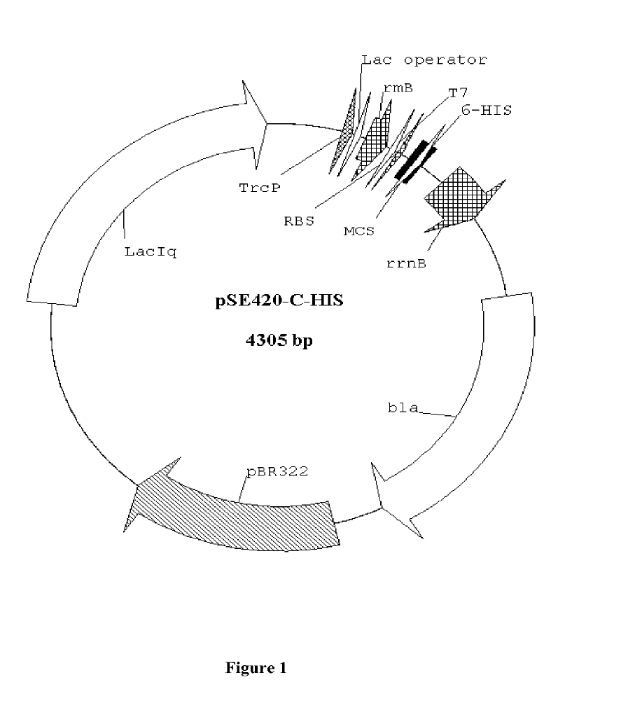

FIG. 1 shows a map of a modified expression vector that was used to produce C-terminally His-tagged constructs for screening. 188 β-glucosidase candidate enzymes were evaluated for activity and thermotolerance at 60° C. E. coli strains expressing the 188 β-glucosidase candidates were picked from glycerol stocks and inoculated into 96-well plates, containing 60 μl LB-carb media comprising about 10 g tryptone, 5 g yeast extract, and 10 g sodium chloride per liter, pH 7.0, with 100 mg/L final concentration of carbenicillin (Sigma, St. Louis, Mo., catalog# C3416). The plates were incubated to allow for growth overnight at about 37° C. The plates were replicated into two separate 384-well plates containing 60 μl LB-carb. One plate was used for activity screening (designated the induction plate) while the other contained about 20 μl of a 60% glycerol-water solution (designated the Gly-stock plate). The Gly-stock plates were then stored at minus 80° C., and were used as the source of inoculum for further testing. The induction plates were grown overnight at about 37° C. Expression of the polypeptide was induced with isopropyl β-D-1-thiogalactopyranoside (IPTG, Invitrogen, Carlsbad, Calif., catalog#15529-019) at about 0.5 mM final concentration, and grown overnight at 30° C.

### Preparation of Lysates Containing β-Glucosidase Candidate Enzymes

The next day following overnight induction of polypeptide expression, cultures were lysed with the addition of about 5 μl of a solution containing about 16 U/ml Benzonase™ (Sigma, St. Louis, Mo., catalog# E1014) and about 3.2 mg/ml Lysozyme (Sigma, St. Louis, Mo., catalog# L6876). The cultures were then subjected to one freeze/thaw cycle at about −80° C. to promote lysis. Lysates were collected for the subsequent β-glucosidase characterizations.

### β-Glucosidase Activity Assay at Various Temperatures

Total protein concentrations of the β-glucosidase lysates samples were measured using the commercial Bio-Rad Protein Assay Dye Reagent Concentrate (Bio-Rad, cat#500-0006, Hercules Calif.). Prior to determination of β-glucosidase activity, two 50 μl aliquots of each lysate were pre-incubated at either 37° C. or 60° C., for 30 minutes. After pre-incubation, β-glucosidase activity was measured using the fluorogenic substrate 4-methylumbelliferyl β-D-glucopyranoside (MUG), (Sigma, St. Louis, Mo., catalog# M3633) in an assay referred to herein as the “MUG assay”. Assays were performed at 37° C., in Costar™ 96-well black bottom plates (Corning Inc., Corning, N.Y., Catalog No. 3631). Wells each contained about 10 μl of lysate and about 40 μl of reaction buffer (50 mM sodium citrate, pH 5.5). Reactions were initiated by the addition of 50 μl MUG (500 μM dissolved in reaction buffer) to a final concentration of about 250 μM. Plates were read using a Spectramax plate reader set at excitation and emission wavelengths of 365 nm and 450 nm respectively, using the kinetic read mode. The linear portion of the kinetic read was used to determine the activity. One unit of activity is defined herein as the liberation of 1.0 μmol of 4-methylumbelliferone (MU) from MUG substrate per minute at around pH 5.5 at about 37° C.

A subset of β-glucosidase candidates was tested for thermotolerance after 30 minute thermal challenges at each of about 60, 70, and 80° C. β-glucosidase activity was then measured using the MUG assay as described herein. To obtain specific activities the approximate concentration of β-glucosidase candidate enzymes was estimated by densitometry of SDS-PAGE gels. The specific activity is represented as units of activity per mg of estimated β-glucosidase enzyme. The results of the thermotolerance test are presented in Table 2. Based on these results, two β-glucosidase candidates having sequences given by SEQ ID NO:378 and SEQ ID NO:379 were selected for evolution using GSSM™ as described in Example 2 below.

TABLE 2

| | Protein | | | | % re- | % re- | % re- |
|---|---|---|---|---|---|---|---|
| | Conc | Protein | Purity | Specific | sidual | sidual | sidual |
| SEQ | Stock | (mg in | (approx- | Activity | (60° | (70° | (80° |
| ID | (mg/ml) | rxn) | imate) | (U/mg) | C.) | C.) | C.) |
| 379 | 0.04 | 0.00004 | 14.0% | 187.4 | 27% | 0% | 1% |
| 378 | 0.25 | 0.00025 | 7.5% | 49.5 | 97% | 1% | 1% |

### Example 2

### Gene Site Saturation Mutagenesis (GSSM™) Library Construction and Screening

A selected β-glucosidase polypeptide (SEQ ID NO:379) was tagged with hexahistidine (6×-His) to facilitate characterization of thermotolerant mutants discovered. Briefly, by using an site-directed mutagenesis (SDM) approach, primers were designed for the purpose of removing the TAA stop codon from the parent BG gene sequence (SEQ ID NO:198), using sequence 20 nucleotides upstream and downstream of the TAA stop codon. Removal of the stop codon allowed translation of the hexahistidine region contained in the pSE420-C-His expression vector (See FIG. 1). The SDM product was then treated with Dpn I enzyme for 4 hours and transformed into One Shot TOP 10 competent cells (Invitrogen, Carlsbad Calif.). Transformants were selected from colonies plated on LB-carb plates, overnight at about 37° C. Plasmids were purified, and BG gene sequences verified using an ABI 3730x1 DNA Analyzer and ABI BigDye® v3.1 cycle sequencing chemistry. The plasmid containing the 6×-His-tagged β-glucosidase polypeptide (SEQ ID NO:380) was mini-prepped, purified, and transformed into a XL1-Blue Competent Cells (200249, Stratagene, La Jolla Calif.). Activity of the expressed β-glucosidase polypeptide was verified using the MUG assay described in Example 1.

The 6×-His-tagged β-glucosidase-encoding gene (SEQ ID NO:380) was mutagenized via GSSM to make the “β-glucosidase GSSM Library.”

The GSSM method is described in U.S. Patent Pub. No. 2009/0220480, pp. 48-50, and was performed with particular modifications as described herein. The β-glucosidase GSSM library was constructed using a 32 codon NNK strategy and transformed into E. coli host XL1-Blue. In the NNK strategy, random peptides are produced by the use of random oligonucleotides for which the codons have the sequence NNK, where N is selected from G, A, T, C and K is selected from G or T.

To accomplish site-saturation mutagenesis every residue of a β-glucosidase enzyme was converted into all 20 amino acids by site directed mutagenesis using 32-fold degenerate oligonucleotide primers. A culture of the β-glucosidase expression construct was grown and a preparation of the plasmid was made. Primers were made to randomize each codon, where the primers have the common structure X₂₀NN(G/T)X₂₀. A reaction mix of 25 μl was prepared containing about 50 ng of plasmid template, 125 ng of each primer, native Pfu polymerase buffer, 200 μM each dNTP and 2.5 units of native Pfu DNA Polymerase. The reaction was cycled in a Perkin-Elmer 9700 thermocycler as follows: Initial denaturation at 95° C. for 3 min, 20 cycles of 95° C. for 45 sec, 50° C. for 45 sec, and 68° C. for 12 min. Final elongation step of 68° C. for 5 min. The reaction mix was digested with 10 units of DpnI at 37° C. for 1 hour to digest the methylated template DNA. 3 μl of each reaction mix were used to transform 50 μl of XL1-Blue cells and the entire transformation mix was plated on large LB-carb plates yielding 200-1000 colonies per plate.

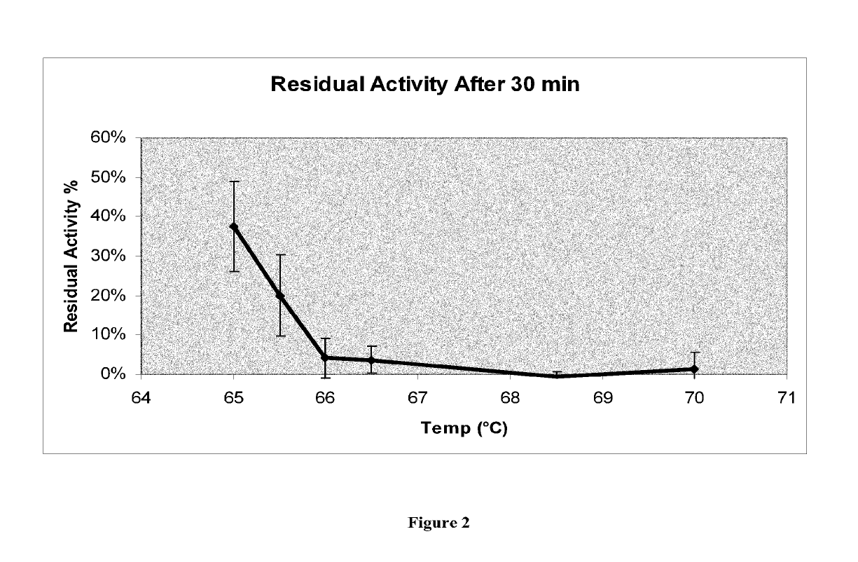

Identification of thermotolerant mutants was carried out by evaluating residual activity using the MUG assay (described in Example 1) following a thermal challenge of 66° C. for 30 minutes. This temperature was selected due to the observation that approximately 5-10% residual activity of the wild-type parent BG (SEQ ID NO:379) remained after a 66° C. challenge (see FIG. 2).

GSSM colonies were grown at 37° C. for about 1 day. Colonies were inoculated into wells of a 384-well plate that contained about 60 μl of LB-carb to generate a master plate (MP). The MP was cultured overnight at 37° C., and was replicated by robotic-pintooling into two separate 384-well plates. One plate contained 60 μl LB-carb per well (designated the induction plate) and the other contained 80 μl LB-carb-10% glycerol for freezing stocks of the library (designated the stock plate). The stock plates were then stored at −80° C. Induction plates were cultured overnight at 37° C. Expression of mutagenized β-glucosidase polypeptides was induced by adding about 20 μl LB-carb containing about 2 mM IPTG and were cultured overnight at 30° C. Cultures were lysed as described previously in Example 1. Lysis plates were stored at −80° C. until assayed.

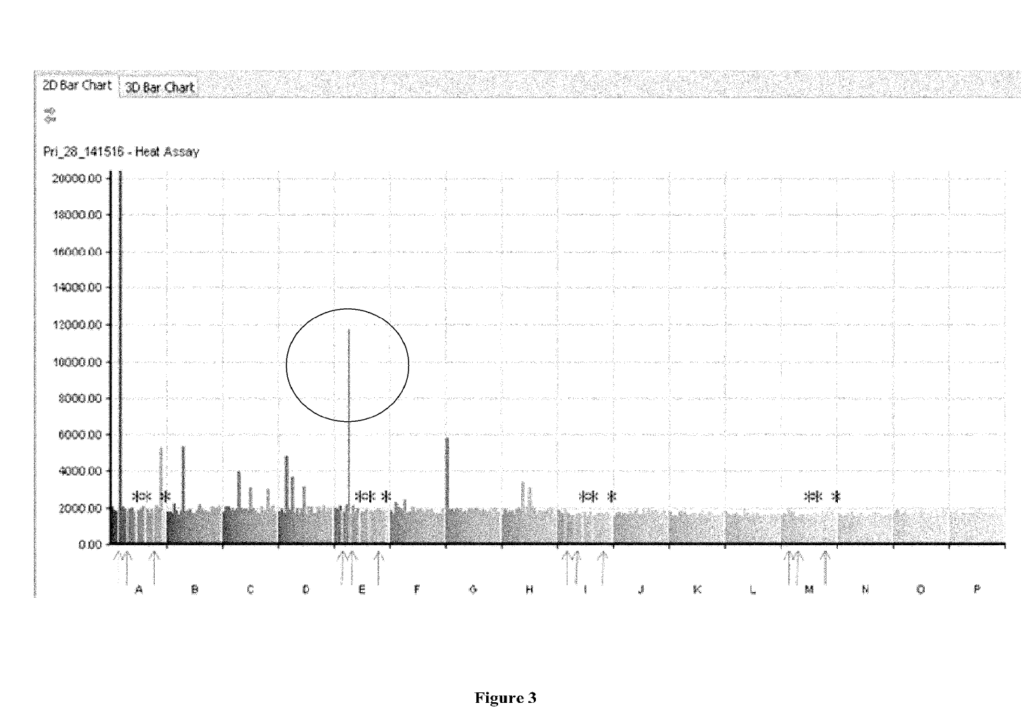

Lysis plates were centrifuged for about 30 minutes at about 4,000 rpm and about 20° C. About 5 μl aliquots of supernatant from the lysis plate were placed into wells of a new plate containing about 75 μl of reaction buffer (designated the dilution plate). To test the library for thermotolerance about 40 μl aliquots of lysate from the dilution plate were placed into wells of a new plate containing about 40 μl of reaction buffer which was heated to about 66° C. for about 30 minutes. About 10 μl of heat-treated lysate was removed and assayed for β-glucosidase activity as described in Example 1, with the modification that the reaction buffer contained 3 mM MUG, pH 5.5 (final volume of 80 μl). After incubation for about 4 minutes, the plates were read using a single read at the wavelengths given in Example 1. As a control, about 10 μl of unheated lysate from the dilution plate was assayed in parallel. Thermotolerant mutants were identified via residual activity (the amount of activity observed in samples after heating compared to activity in the un-heated sample of the same lysate), in comparison to activity of a control (SEQ ID NO:380). An example of the GSSM library screening plate results are presented in FIG. 3.

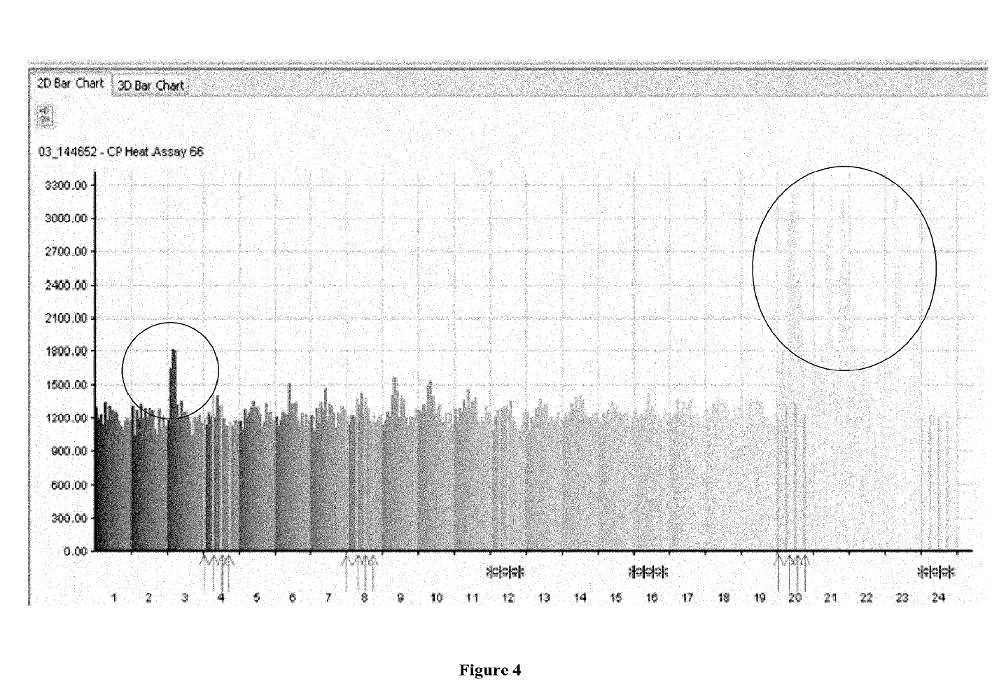

Putative thermotolerant mutants were re-confirmed by repeating the assay procedure above with mutants picked in triplicate and re-assayed for residual activity as described for GSSM screening, (see FIG. 4 for an example of triplicate re-assay data).

To further differentiate the mutants, additional plates containing GSSM mutants were challenged for 30 minutes at two temperatures, about 66° C. and 70° C. Residual activity for the mutants was determined using the method described in Example 1, and the data are displayed in Table 3 below. The asterisk (*) indicates a stop codon.

TABLE 3
Secondary Thermotolerance Screen of GSSM mutants: (Activity, Residual activity,
thermal challenges, at 66° C. and 70° C., at pH 5.5)

| | Activity | Activity | Activity | | | | |
|---|---|---|---|---|---|---|---|
| | (U/mg Protein) @ | (U/mg Protein), | (U/mg Protein), | % | % | | AA-mutations |
| SEQ | Ambient | (post 66° C. | (post 70° C. | Residual | Residual | AA | and position |
| ID | (~22 C.) | challenge) | challenge) | (66° C.) | (70° C.) | SEQID | (polypeptide) |
| 1 | 25.93 | 0.21 | 0.03 | 1% | 0% | 199 | A410R |
| 2 | 26.59 | 0.04 | −0.01 | 0% | 0% | 200 | A410T K451Q |
| 3 | 24.49 | 0.01 | −0.02 | 0% | 0% | 201, 380 | |
| 4 | 25.07 | 0.04 | 0.02 | 0% | 0% | 202 | A449* |
| 5 | 28.88 | 1.17 | 0.11 | 4% | 0% | 203 | A449C |
| 6 | 20.76 | 0.00 | 0.00 | 0% | 0% | 204 | A449D |
| 7 | 29.79 | 0.10 | 0.01 | 0% | 0% | 205 | A68F |
| 8 | 22.73 | 0.31 | −0.01 | 1% | 0% | 206 | A68W |
| 9 | 25.77 | 0.45 | 0.01 | 2% | 0% | 207 | A73G |
| 10 | 23.80 | 0.46 | 0.00 | 2% | 0% | 208 | A73S |
| 11 | 32.13 | 0.58 | 0.00 | 2% | 0% | 208 | A73S |
| 12 | 30.12 | 0.49 | 0.00 | 2% | 0% | 208 | A73S |
| 13 | 28.70 | 0.02 | 0.00 | 0% | 0% | 209 | D302S |
| 14 | 11.92 | 0.03 | 0.01 | 0% | 0% | 210 | D414S |
| 15 | 10.26 | 0.10 | 0.07 | 1% | 1% | 211 | D414T |
| 16 | 10.06 | 0.12 | 0.03 | 1% | 0% | 211 | D414T |
| 17 | 31.02 | 0.00 | −0.01 | 0% | 0% | 212 | D7G |
| 18 | 24.12 | 0.01 | −0.01 | 0% | 0% | 212 | D7G |
| 19 | 28.76 | 0.02 | −0.02 | 0% | 0% | 213 | D7H |
| 20 | 27.96 | 0.00 | −0.02 | 0% | 0% | 214 | E105G |
| 21 | 16.25 | 0.01 | 0.01 | 0% | 0% | 215 | E428D |
| 22 | 13.14 | −0.01 | 0.00 | 0% | 0% | 216 | E428I |
| 23 | 11.19 | 0.09 | 0.04 | 1% | 0% | 217 | E450C |
| 24 | 9.58 | 1.94 | 0.01 | 20% | 0% | 218 | F292I |
| 25 | 12.19 | 0.70 | 0.00 | 6% | 0% | 219 | F292V |
| 26 | 20.01 | 4.50 | 0.02 | 22% | 0% | 220 | T219A |
| 27 | 26.61 | 0.03 | −0.01 | 0% | 0% | 221 | A449E |
| 28 | 12.28 | 0.09 | −0.01 | 1% | 0% | 222 | H315F |
| 29 | 26.37 | 0.08 | 0.04 | 0% | 0% | 223 | H315I |
| 30 | 28.22 | 0.14 | 0.02 | 0% | 0% | 224 | H315K |
| 31 | 5.60 | 4.78 | 2.54 | 85% | 45% | 225 | I216V T219A |
| | | | | | | | M246K |
| 32 | 31.81 | 0.03 | 0.00 | 0% | 0% | 226 | I378L |
| 33 | 31.69 | 0.15 | 0.00 | 0% | 0% | 227 | I63V |
| 34 | 48.64 | 0.34 | −0.01 | 1% | 0% | 227 | I63V |
| 35 | 25.38 | 0.02 | 0.01 | 0% | 0% | 228 | K415A |
| 36 | 24.70 | 0.03 | 0.04 | 0% | 0% | 229 | K451D |
| 37 | 14.51 | 0.00 | 0.00 | 0% | 0% | 230 | K6V |
| 37 | 14.90 | 0.00 | −0.01 | 0% | 0% | 230 | K6V |
| 38 | 8.45 | 0.10 | 0.02 | 1% | 0% | 231 | L427G |
| 39 | 6.91 | 0.08 | −0.03 | 1% | 0% | 231 | L427G |
| 40 | 21.84 | 0.06 | 0.01 | 0% | 0% | 232 | L447F |
| 41 | 24.17 | 12.49 | 0.65 | 52% | 3% | 233 | M246H |
| 42 | 22.13 | 3.78 | 0.05 | 17% | 0% | 234 | M246K |
| 43 | 19.32 | 0.30 | 0.00 | 2% | 0% | 235 | M325T |
| 44 | 18.28 | 0.22 | 0.01 | 1% | 0% | 235 | M325T |
| 45 | 11.77 | 0.02 | 0.02 | 0% | 0% | 236 | N175S |
| 46 | 27.82 | 0.67 | 0.00 | 2% | 0% | 237 | N326G |
| 47 | 29.05 | 0.63 | 0.01 | 2% | 0% | 237 | N326G |
| 48 | 18.34 | 0.03 | 0.00 | 0% | 0% | 238 | N332E |
| 49 | 27.62 | 0.01 | −0.01 | 0% | 0% | 239 | Q139P |
| 50 | 19.56 | 0.01 | 0.02 | 0% | 0% | 240 | Q142K |
| 51 | 22.72 | 0.01 | 0.00 | 0% | 0% | 241 | Q366N |
| 52 | 22.81 | −0.03 | 0.01 | 0% | 0% | 242 | Q3E |
| 53 | 28.40 | 0.03 | 0.01 | 0% | 0% | 243 | Q3M |
| 54 | 23.42 | 0.00 | −0.01 | 0% | 0% | 244 | Q3R |
| 55 | 28.36 | −0.02 | −0.01 | 0% | 0% | 245 | R416T |
| 56 | 30.37 | 4.83 | 0.02 | 16% | 0% | 246 | S296T |
| 57 | 13.66 | 0.02 | −0.01 | 0% | 0% | 247 | S317K |
| 58 | 26.96 | 0.01 | 0.00 | 0% | 0% | 201 | |
| 59 | 16.53 | 0.00 | 0.01 | 0% | 0% | 248 | S402T |
| 60 | 14.94 | 3.27 | 0.02 | 22% | 0% | 220 | T219A |
| 61 | 21.14 | 3.89 | 0.01 | 18% | 0% | 220 | T219A |
| 62 | 13.19 | 1.91 | 0.01 | 14% | 0% | 249 | T219S |
| 63 | 27.10 | 0.00 | −0.01 | 0% | 0% | 250 | T24D |
| 64 | 20.15 | 0.02 | −0.01 | 0% | 0% | 251 | T24H |
| 65 | 25.71 | 0.04 | 0.00 | 0% | 0% | 252 | T297S |
| 66 | 28.04 | 0.79 | 0.01 | 3% | 0% | 253 | T441V |
| 67 | 33.52 | 0.47 | −0.01 | 1% | 0% | 253 | T441V |
| 68 | 26.04 | 0.45 | 0.02 | 2% | 0% | 254 | V167A K231E |
| 69 | 0.03 | −0.01 | 0.00 | −29% | −5% | 255 | V203P |
| 70 | 18.66 | −0.04 | −0.01 | 0% | 0% | 256 | V420L |
| 71 | 27.91 | 0.02 | −0.01 | 0% | 0% | 257 | V60R |
| 72 | 20.65 | 7.43 | 0.28 | 36% | 1% | 258 | W401F |
| 73 | 29.37 | 3.42 | 0.00 | 12% | 0% | 259 | Y399F |
| 74 | 13.74 | −0.02 | 0.00 | 0% | 0% | 201 | |
| 75 | 23.48 | 4.12 | 0.02 | 18% | 0% | 246 | S296T |
| 76 | 11.83 | 0.51 | −0.01 | 4% | 0% | 260 | Y74L |
| 198 | 26.48 | 0.01 | −0.01 | 0% | 0% | 380, 201 | Parent |

In a tertiary thermotolerance screen, a subset of the GSSM library mutants including V203P (SEQ ID NO:255), M246H (SEQ ID NO:233), and the triple mutant I216V-T219A-M246K (SEQ ID NO:225) were evaluated for residual MUG activity after increased thermal challenges (66, 70, and 80° C.) and compared against the wild-type polypeptides (SEQ ID NO:380 and SEQ ID NO:378) and a commercial β-glucosidase benchmark (Cellobiase, Sigma, St Louis, Mo., Catalog# C6105). As shown in Table 4, the triple mutant (I216V-T219A-M246K) showed activity after the 70° C. thermal challenge, while the wild-type polypeptide and commercial benchmark did not have any observable activity after the same challenge.

TABLE 4
(Activity of 3 thermostable GSSM mutants, with WT-
(SEQID380) and a commercial benchmark Cellobiase)

| | | RT - | 66° C. - | 70° C. - | 80° C. - | | | |
|---|---|---|---|---|---|---|---|---|
| | | Activity | Activity | Activity | Activity | Residual | Residual | Residual |
| | Sample | (U/mg | (U/mg | (U/mg | (U/mg | Activity | Activity | Activity |
| SEQ ID | Description | protein) | protein) | protein) | protein) | (66° C.) | (70° C.) | (80° C.) |
| 255 | V203P | 0.253 | 0.064 | 0.006 | 0.000 | 25.1% | 2.5% | 0.0% |
| 233 | M246H | 28.398 | 23.106 | 1.931 | −0.024 | 81.4% | 6.8% | ND |
| 225 | I216V, | 10.921 | 6.589 | 4.234 | −0.014 | 60.3% | 38.8% | ND |
| | T219A, | | | | | | | |
| | M246K | | | | | | | |
| 378 | 13473 | 3.751 | 0.013 | 0.000 | 0.004 | 0.4% | ND | 0.1% |
| 380 | 27394 | 23.088 | 0.063 | 0.038 | 0.001 | 0.3% | 0.2% | 0.0% |
| Cb'ase | Commercial | 1.969 | 0.034 | 0.003 | 0.002 | 1.7% | 0.1% | 0.1% |
| | BG | | | | | | | |

Total protein and purity for a subset of GSSM mutants (V203P, M246H, and the triple mutant (I216V, T219A, M246K) was determined as described in Example 1, and the specific activity (SA) on MUG after 3 thermal challenges (66° C., 70° C., and 80° C. for 30 min), was determined. The specific activity of the triple mutant (I216V-T219A-M246K, SEQ ID NO:255) remained the highest after the 70° C. thermal challenge, while the parent β-glucosidases (SEQ ID NO:378 and SEQ ID NO:380) did not have any observable activity after the same challenge. V203P resulted in an almost complete loss of activity. Results are summarized in Table 5, below.

TABLE 5
Specific Activity (SA) of thermotolerant mutants (U/mg enzyme)

| | | RT - | 66° C. - | 70° C. - | 80° C. - |
|---|---|---|---|---|---|
| | Sample | Specific | Specific | Specific | Specific |
| | De- | Activity | Activity | Activity | Activity |
| SEQ | scrip- | (U/mg EN- | (U/mg EN- | (U/mg EN- | (U/mg EN- |
| ID | tion | ZYME) | ZYME) | ZYME) | ZYME) |
| 233 | M246H | 94.659 | 77.021 | 6.437 | −0.079 |
| 225 | I216V, | 36.404 | 21.963 | 14.112 | −0.045 |
| | T219A, | | | | |
| | M246K | | | | |
| 378 | 13473 | 16.039 | 0.057 | −0.001 | 0.017 |
| 380 | 27394 | 69.965 | 0.192 | 0.116 | 0.004 |

### Example 3

### Further Evolution of Thermotolerance, Via TMCA-Reassembly Method

Thirteen of the amino acid mutations identified from the GSSM screen and listed in Tables 6A and 6B were chosen for combinatorial assembly using the TMCA-reassembly method. This method is described in WO2009/018449 and involves annealing multiple mutagenic oligonucleotides to a denatured double-stranded vector template. Upon digestion of parental DNA, the result is a mixture of clones containing various combinations of mutations, referred to as the “reassembly library.”

Two different parental polypeptides were used to generate two reassembly libraries. For the first library (“Reassembly Library 1”), 13 amino acid changes were reassembled into the original parental β-glucosidase polypeptide (SEQ ID NO:380) and for the second library (“Reassembly Library 2”), 10 amino acid changes were selected for reassembly in an alternate β-glucosidase parental polypeptide (SEQ ID NO:378).

TABLE 6A
Reassembly Library for original parental
β-glucosidase (SEQ ID NO: 380)

| Original | New | Original | New | Codon Mutation | |
|---|---|---|---|---|---|
| Codon | Codon | AA | AA | Location | Mutation |
| GAT | CAT | D | H | 7 | D7H |
| TAT | TTG | Y | L | 74 | Y74L |
| ATT | GTT | I | V | 216 | I216V |
| ACG | GCG | T | A | 219 | T219A |
| ACG | AGT | T | S | 219 | T219S |
| ATG | AAG | M | K | 246 | M246K |
| ATG | CAT | M | H | 246 | M246H |
| TTT | GTT | F | V | 292 | F292V |
| TTT | ATT | F | I | 292 | F292I |
| TCA | ACT | S | T | 296 | S296T |
| TAT | TTT | Y | F | 399 | Y399F |
| GTA | TAT | V | Y | 400 | V400Y |
| TGG | TTT | W | F | 401 | W401F |

TABLE 6B
Reassembly Library for alternate parental
β-glucosidase (SEQ ID NO: 378)

| Original | New | Original | New | Codon Mutation | |
|---|---|---|---|---|---|
| Codon | Codon | AA | AA | Location | Mutation |
| GAC | CAT | D | H | 11 | D11H |
| TAT | TTG | Y | L | 78 | Y78L |
| ACC | GCG | T | A | 222 | T222A |
| ACC | AGT | T | S | 222 | T222S |
| TTC | CAT | F | H | 248 | F248H |
| TTC | AAG | F | K | 248 | F248K |
| ATC | GTT | I | V | 293 | I292V |
| TCG | ACT | S | T | 297 | S297T |
| GCC | TAT | A | Y | 398 | A398Y |
| TGG | TTT | W | F | 399 | W399F |

Variants from the TMCA-reassembly libraries derived from SEQ ID NO:380 and SEQ ID NO:378 were screened for thermotolerance as described in Example 1, with the following changes: Induction plates from both libraries were cultured at 30° C. overnight; plates from SEQ ID NO:380 library were heated to about 84, 86, 88° C. for 30 minutes; plates from SEQ ID NO:378 library were heated to about 68, 69, and 70° C. for 30 minutes. Residual MUG activity was determined for both libraries as described in Example 1, with the following changes: for the SEQ ID NO:378 reassembly library, the residual activity was determined using the activity of the 68° C. challenged sample as the reference activity, so residual activity listed is percent residual activity of the 68° C. samples. Results for the reassembly library with a β-glucosidase of SEQ ID NO:380 are summarized in Table 7, below. Results for the reassembly library with a β-glucosidase of SEQ ID NO:378 are summarized in Table 8, below.

TABLE 7

| | | Residual | Residual | Residual | Parent amino acid and position (Column Header), reassembly variants with | | | | | | | | | | | | |
|---|---|---|---|---|---|---|---|---|---|---|---|---|---|---|---|---|---|
| SEQID- | SEQID- | Activity | Activity | Activity | amino acid changes, listed in rows | | | | | | | | | | | | |
| DNA | AA | (84° C.) | (86° C.) | (88° C.) | D7 | Y74 | D154 | G200 | I216 | T219 | M246 | P253 | F292 | S296 | Y399 | V400 | W401 |
| 77 | 261 | 52.3% | 18.0% | ND | H | L | | | V | A | H | | V | T | | | |
| 78 | 262 | 48.4% | 12.2% | ND | H | L | | | V | A | K | | I | T | | | |
| 79 | 263 | 10.3% | ND | ND | H | L | | | V | A | | | I | T | | | |
| 80 | 264 | 12.5% | ND | ND | H | L | | | V | A | | | V | T | | | |
| 81 | 265 | 38.1% | ND | ND | H | L | | | V | S | K | | I | T | | Y | |
| 82 | 266 | 10.6% | ND | ND | H | L | | | V | S | | | I | T | | Y | |
| 83 | 267 | 35.8% | 95.4% | ND | H | L | | | V | S | | | V | T | | Y | |
| 84 | 268 | ND | ND | ND | H | L | | | V | | K | | V | T | | Y | |
| 85 | 269 | 61.4% | ND | ND | H | L | | | | A | K | | I | T | | | |
| 86 | 270 | 21.3% | 7.8% | ND | H | L | | | | S | K | | I | T | | | |
| 87 | 271 | 22.4% | ND | ND | H | L | | | | S | K | | V | T | | | |
| 88 | 272 | ND | ND | ND | H | L | | | | | H | | V | T | F | Y | |
| 89 | 273 | 16.5% | ND | ND | H | | | | V | A | H | | V | T | | | |
| 90 | 274 | ND | ND | ND | H | | | | V | A | H | | | T | F | Y | |
| 91 | 275 | 17.0% | 3.2% | ND | H | | | | V | A | K | | I | T | F | Y | |
| 92 | 276 | 34.7% | 13.3% | ND | H | | | | V | A | K | | I | T | | | |
| 93 | 277 | 8.0% | ND | ND | H | | | | V | A | K | | | T | | | F |
| 94 | 278 | 26.4% | 5.3% | ND | H | | | | V | A | | | V | T | F | | F |
| 95 | 279 | 13.0% | 3.2% | ND | H | | | | V | A | | | V | T | | | F |
| 96 | 280 | 39.8% | 15.4% | ND | H | | | | V | S | H | | I | T | | | F |
| 97 | 281 | 23.3% | ND | ND | H | | | | V | S | H | | I | T | | Y | |
| 98 | 282 | 35.0% | 13.0% | 6.7% | H | | | | V | S | H | | V | T | F | | F |
| 99 | 283 | 42.0% | 13.8% | ND | H | | | | V | S | K | | I | T | | | F |
| 100 | 284 | 20.0% | 5.6% | ND | H | | | | V | S | K | | V | T | | Y | |
| 101 | 285 | 13.0% | 3.1% | ND | H | | | | V | S | | | I | T | F | | F |
| 102 | 286 | 9.4% | ND | ND | H | | | | V | S | | | I | T | F | Y | |
| 103 | 287 | 18.3% | 2.9% | ND | H | | | | V | S | | | I | T | | | F |
| 104 | 288 | 17.7% | 4.3% | ND | H | | | | V | S | | | V | T | F | | F |
| 105 | 289 | 13.6% | 2.6% | ND | H | | | | V | S | | | V | T | | | F |
| 106 | 290 | 12.8% | 3.2% | ND | H | | | | V | | K | | I | T | F | | F |
| 107 | 291 | 9.4% | ND | ND | H | | | | V | | K | | V | T | | | F |
| 108 | 292 | 69.2% | 28.4% | ND | H | | | | | A | H | | V | T | F | Y | |
| 109 | 293 | 46.3% | 10.8% | ND | H | | | | | A | H | | V | T | | Y | |
| 110 | 294 | 27.5% | ND | ND | H | | | | | A | K | | V | T | F | Y | |
| 111 | 295 | 23.6% | 5.1% | ND | H | | | | | A | | | I | T | F | | F |
| 112 | 296 | 14.6% | 2.8% | ND | H | | | | | A | | | I | T | | | F |
| 113 | 297 | 21.0% | 3.9% | ND | H | | | | | A | | | V | T | F | | F |
| 114 | 298 | 13.3% | ND | ND | H | | | | | S | H | | I | T | | Y | |
| 115 | 299 | 28.8% | 7.9% | ND | H | | | | | S | K | | I | T | | | F |
| 116 | 300 | 10.9% | 3.0% | ND | H | | | | | S | K | | I | T | | Y | |
| 117 | 301 | 21.1% | 3.6% | ND | H | | | | | S | K | | V | T | F | Y | |
| 118 | 302 | 3.5% | ND | ND | H | | | | | S | K | | | T | | | F |
| 119 | 303 | 18.9% | 4.1% | ND | H | | | | | S | | | I | T | F | | F |
| 119 | 303 | 11.8% | ND | ND | H | | | | | S | | | I | T | F | | F |
| 120 | 304 | 25.7% | 4.9% | ND | H | | | | | S | | P | V | T | F | | F |
| 121 | 304 | 21.7% | 8.2% | ND | H | | | | | S | | | V | T | F | | F |
| 122 | 305 | 7.1% | ND | ND | H | | | | | S | | | V | T | | | F |
| 123 | 306 | 5.8% | ND | ND | H | | | | | | H | | V | T | F | | F |
| 124 | 307 | 19.6% | 2.3% | ND | H | | | | | | K | | I | T | F | | F |
| 125 | 308 | 5.6% | ND | ND | | L | | | V | A | | | I | T | | | |
| 126 | 309 | 68.0% | 17.1% | ND | | L | | | V | A | | | V | T | | Y | |
| 127 | 310 | 23.2% | 5.5% | ND | | L | | | V | A | | | V | T | | | |
| 128 | 311 | ND | ND | ND | | L | | | V | S | K | | I | T | | | |
| 129 | 312 | 22.3% | 6.3% | ND | | L | | | V | S | K | | V | T | | | |
| 130 | 313 | 36.6% | ND | ND | | L | | | V | S | | | I | T | F | Y | |
| 131 | 314 | 22.5% | 4.0% | ND | | L | | | V | | K | | V | T | | Y | |
| 132 | 315 | 7.0% | ND | ND | | L | | | | A | | | I | T | | | |
| 133 | 316 | 38.1% | 6.2% | ND | | L | | | | A | | | V | T | | | |
| 134 | 317 | 28.5% | ND | ND | | L | | | | S | K | | I | T | | | |
| 135 | 318 | 15.2% | 4.9% | ND | | L | | | | S | | | V | T | | Y | |
| 136 | 319 | 6.8% | ND | ND | | | | | V | A | H | | I | T | F | | |
| 137 | 320 | 49.7% | 19.3% | 6.7% | | | | | V | A | H | | V | T | | | F |
| 138 | 321 | 9.4% | ND | ND | | | | | V | A | K | | I | T | | | |
| 139 | 322 | 57.1% | 22.7% | 6.3% | | | | | V | A | K | | V | T | F | | F |
| 31 | 225 | ND | 2.5% | ND | | | | | V | A | K | | | | | | |
| 140 | 323 | 23.3% | 3.9% | ND | | | | | V | A | | | I | T | F | | F |
| 141 | 324 | 8.7% | ND | ND | | | | | V | A | | | I | T | F | Y | |
| 142 | 325 | 7.9% | ND | ND | | | | | V | A | | | V | T | F | Y | |
| 143 | 326 | 18.3% | 4.0% | ND | | | | | V | A | | | V | T | | | F |
| 144 | 327 | 24.1% | 5.2% | ND | | | | | V | S | H | | I | T | F | | F |
| 145 | 328 | 28.0% | 5.4% | ND | | | | | V | S | K | | I | T | F | | F |
| 146 | 329 | 53.2% | 8.8% | ND | | | | | V | S | K | | I | T | F | Y | |
| 147 | 330 | 31.4% | 8.9% | ND | | | | | V | S | K | | I | T | | | F |
| 148 | 331 | 24.4% | 7.9% | ND | | | | | V | S | K | | I | T | | Y | |
| 149 | 332 | 22.6% | 6.5% | ND | | | | | V | S | K | | V | T | | | F |
| 150 | 333 | ND | ND | ND | | | N | | V | S | K | | V | | F | | F |
| 151 | 334 | 21.3% | 4.7% | ND | | | | | V | S | | | I | T | F | | F |
| 152 | 335 | ND | ND | ND | | | | | V | S | | | I | T | F | Y | |
| 153 | 336 | 17.6% | 4.0% | ND | | | | | V | S | | | V | T | | | F |
| 154 | 337 | 3.1% | ND | ND | | | | | V | | H | | I | T | F | | F |
| 155 | 338 | 12.2% | ND | ND | | | | | V | | K | | I | T | F | | F |
| 156 | 339 | 12.0% | ND | ND | | | | | V | | K | | I | T | | | F |
| 157 | 340 | 34.5% | 6.0% | ND | | | | | | A | K | | I | T | F | Y | |
| 158 | 341 | 27.1% | 29.4% | ND | | | | | | A | K | | I | T | | Y | |
| 159 | 342 | 31.4% | 7.4% | ND | | | | | | A | K | | V | T | | Y | |
| 160 | 343 | 19.2% | 3.4% | ND | | | | | | A | | | I | T | F | | F |
| 161 | 344 | 25.8% | 8.4% | ND | | | | | | A | | | V | T | | | F |
| 162 | 345 | 33.7% | ND | ND | | | | | | S | H | | I | T | | Y | |
| 163 | 346 | 34.1% | 7.4% | ND | | | | | | S | K | | I | T | F | Y | |
| 164 | 347 | 29.9% | 11.1% | ND | | | | | | S | K | | I | T | | | F |
| 165 | 348 | 12.4% | ND | ND | | | | | | S | K | | I | T | | Y | |
| 166 | 349 | 28.0% | ND | ND | | | | | | S | K | | V | T | F | Y | |
| 167 | 350 | 27.0% | 3.7% | ND | | | | | | S | | | I | T | F | | F |
| 168 | 351 | 12.8% | ND | ND | | | | | | S | | | V | T | F | | F |
| 169 | 352 | ND | ND | ND | | | | G | | S | | | V | T | F | Y | |
| 170 | 353 | 25.0% | 3.0% | ND | | | | | | | K | | I | T | | | F |
| 171 | 354 | 7.0% | ND | ND | | | | | | | K | | V | T | | | F |
| 172 | 355 | ND | ND | ND | | | | | | | | | I | T | F | | F |

TABLE 8

| | | ND = no residual activity detected) | | WT AA (listed in column header), | | | | | | | | | | |
|---|---|---|---|---|---|---|---|---|---|---|---|---|---|---|
| SEQID- | SEQID- | Avg % residual activity | Avg % residual activity | reassembly variant with AA changes listed in rows | | | | | | | | | | |
| DNA | AA | (68-69° C.) | (68-70° C.) | D11 | N96 | T222 | D245 | F248 | I293 | S297 | H303 | L309 | R315 | D363 |
| 173 | 356 | 37.2% | 7.9% | | | A | | H | | T | | | | G |
| 174 | 357 | 29.2% | 7.4% | H | | | | H | V | T | | | H | |
| 175 | 358 | 34.7% | 12.6% | | | A | | H | | T | | L | | |
| 176 | 359 | 44.7% | 9.5% | | | | | H | | T | R | | | |
| 177 | 360 | 34.8% | ND | H | | | H | H | V | T | | | | |
| 178 | 361 | 39.2% | 8.7% | H | | A | | H | V | T | | | | |
| 179 | 362 | 41.6% | 7.2% | | | A | | H | V | T | | | | |
| 180 | 363 | 37.7% | 9.0% | H | | S | | H | V | T | | | | |
| 181 | 364 | 25.7% | 6.4% | | | S | | H | V | T | | | | |
| 182 | 365 | 36.2% | 7.2% | H | | | | H | V | T | | | | |
| 183 | 366 | 39.1% | 6.4% | | | | | H | V | T | | | | |
| 184 | 367 | 40.7% | 13.7% | | | A | | K | V | T | | | | |
| 185 | 368 | 28.1% | 7.9% | | | S | | K | V | T | | | | |
| 186 | 369 | 28.3% | 4.8% | | | | | K | V | T | | | | |
| 187 | 370 | 31.6% | ND | | | A | | | V | T | | | | |
| 188 | 371 | 28.5% | ND | | | | H | H | | T | | | | |
| 189 | 372 | 41.5% | 8.5% | H | | A | | H | | T | | | | |
| 190 | 358 | 34.5% | 7.2% | | | A | | H | | T | | | | |
| 191 | 373 | 24.8% | 6.5% | | | S | | H | | T | | | | |
| 192 | 374 | 36.9% | ND | | N | | | H | | T | | | | |
| 193 | 375 | 37.7% | ND | H | | | | H | | T | | | | |
| 194 | 374 | 38.0% | 5.9% | | | | | H | | T | | | | |
| 195 | 376 | 25.7% | ND | H | | | | K | | T | | | | |
| 196 | 377 | 27.7% | 6.8% | | | | | K | | T | | | | |

TABLE 9A

| Corresp. | Sequence Name | | | | | | | | |
|---|---|---|---|---|---|---|---|---|---|
| Sequence Pos. | Q3 | K6 | D7 | T24 | V60 | I63 | A68 | A73 | Y74 |
| SEQ ID NO. | Q-3 | K-6 | D-7 | T-24 | V-60 | I-63 | A-68 | A-73 | Y-74 |
| 379/380 | | | | | | | | | |
| SEQ ID NO. 378 | Q-4 | A-10 | D-11 | D-28 | R-64 | V-67 | E-72 | A-77 | Y-78 |
| US8101393-0094 | — | K-6 | D-7 | T-24 | V-60 | I-63 | A-68 | A-73 | Y-74 |
| US8101393-0388 | — | K-6 | D-7 | T-24 | V-60 | I-63 | A-68 | A-73 | Y-74 |
| US8101393-0172 | Q-4 | A-10 | D-11 | D-28 | R-64 | V-67 | E-72 | G-77 | Y-78 |
| JP2011205992-0018 | A-2 | A-8 | D-9 | R-26 | G-62 | I-65 | A-70 | A-75 | Y-76 |
| JP2011205992-0023 | K-2 | A-8 | N-9 | S-26 | P-62 | I-65 | E-70 | A-75 | Y-76 |
| JP2011205992-0022 | A-2 | P-8 | R-9 | T-26 | Q-62 | V-65 | N-70 | A-75 | Y-76 |
| US20110214199- | D-4 | H-10 | D-11 | A-28 | R-64 | I-67 | Q-72 | A-77 | Y-78 |
| 58656 | | | | | | | | | |
| US20110214199- | D-20 | D-26 | D-27 | A-44 | H-80 | I-83 | R-88 | A-93 | Y-94 |
| 62406 | | | | | | | | | |
| US20110214199- | — | N-6 | D-7 | S-24 | R-60 | V-63 | W-68 | S-73 | Y-74 |
| 47919 | | | | | | | | | |
| US20110214199- | — | E-5 | G-6 | G-23 | E-59 | L-62 | R-67 | A-72 | Y-73 |
| 60662 | | | | | | | | | |
| US20110214199- | — | D-12 | G-13 | T-30 | R-66 | V-69 | D-74 | H-79 | Y-80 |
| 36660 | | | | | | | | | |
| US20110214199- | — | P-18 | A-19 | R-36 | R-72 | V-75 | E-80 | A-85 | Y-86 |
| 17908 | | | | | | | | | |
| US20110214199- | — | E-6 | K-7 | Q-24 | E-60 | I-63 | S-68 | V-73 | Y-74 |
| 25308 | | | | | | | | | |
| US20110214199- | — | E-6 | K-7 | Q-24 | E-60 | I-63 | S-68 | A-73 | Y-74 |
| 64004 | | | | | | | | | |
| US20110214199- | P-18 | S-24 | D-25 | T-42 | R-78 | V-81 | E-86 | A-91 | Y-92 |
| 25023 | | | | | | | | | |
| US20110214199- | — | E-6 | K-7 | Q-24 | E-60 | I-63 | S-68 | V-73 | Y-74 |
| 52644 | | | | | | | | | |
| US20110214199- | — | P-15 | A-16 | R-33 | R-69 | V-72 | D-77 | A-82 | Y-83 |
| 13862 | | | | | | | | | |
| US20110214199- | E-3 | K-9 | D-10 | N-27 | K-63 | V-66 | E-71 | A-76 | Y-77 |
| 29446 | | | | | | | | | |
| US20110214199- | — | E-6 | K-7 | Q-24 | E-60 | I-63 | S-68 | A-73 | Y-74 |
| 5988 | | | | | | | | | |
| US20110214199- | — | E-6 | K-7 | Q-24 | E-60 | I-63 | S-68 | A-73 | Y-74 |
| 23246 | | | | | | | | | |
| US20110214199- | E-2 | K-8 | D-9 | K-26 | K-62 | V-65 | S-70 | S-75 | Y-76 |
| 12000 | | | | | | | | | |
| US20110214199- | R-3 | K-9 | D-10 | N-27 | K-63 | I-66 | E-71 | A-76 | Y-77 |
| 56949 | | | | | | | | | |
| US8101393-0272 | — | A-18 | G-19 | T-36 | D-72 | V-75 | R-80 | D-85 | Y-86 |
| US8101393-0014 | T-6 | A-12 | G-13 | D-30 | R-66 | V-69 | G-74 | A-79 | Y-80 |
| US8101393-0398 | A-17 | P-23 | G-24 | A-41 | S-77 | V-80 | E-85 | S-90 | Y-91 |
| US8101393-0492 | A-17 | P-23 | G-24 | A-41 | S-77 | V-80 | E-85 | S-90 | Y-91 |
| US8101393-0400 | P-17 | P-23 | G-24 | T-41 | P-77 | V-80 | D-85 | S-90 | Y-91 |
| US8101393-0266 | — | A-17 | D-18 | A-35 | R-71 | V-74 | E-79 | A-84 | Y-85 |
| US8101393-0366 | — | E-4 | G-5 | G-22 | R-58 | L-61 | W-66 | A-71 | Y-72 |
| US8101393-0342 | T-2 | S-8 | N-9 | N-26 | R-62 | V-65 | D-70 | A-75 | Y-76 |
| US8101393-0356 | T-2 | K-8 | G-9 | R-26 | P-62 | I-65 | A-70 | G-75 | Y-76 |
| US8101393-0320 | S-2 | K-8 | D-9 | N-26 | E-62 | I-65 | E-70 | S-75 | Y-76 |
| US7314974-19451 | — | N-4 | D-5 | S-22 | R-58 | V-61 | W-66 | S-71 | Y-72 |
| US7314974-8251 | — | — | — | T-17 | R-53 | V-56 | E-61 | A-66 | Y-67 |
| US7630836-9340 | D-4 | H-10 | D-11 | A-28 | R-64 | I-67 | Q-72 | A-77 | Y-78 |
| US7630836-12787 | — | P-18 | A-19 | R-36 | R-72 | V-75 | E-80 | A-85 | Y-86 |
| US20090220480- | — | D-6 | N-7 | R-24 | G-60 | V-63 | W-68 | A-73 | Y-74 |
| 0018 | | | | | | | | | |
| US20120034253- | — | E-6 | K-7 | Q-24 | E-60 | I-63 | S-68 | V-73 | Y-74 |
| 0080 | | | | | | | | | |
| US20120034253- | — | E-6 | K-7 | Q-24 | E-60 | I-63 | S-68 | A-73 | Y-74 |
| 0077 | | | | | | | | | |
| US20120034253- | — | E-6 | K-7 | Q-24 | E-60 | I-63 | S-68 | A-73 | Y-74 |
| 0078 | | | | | | | | | |
| WO2012016960- | — | D-36 | G-37 | R-54 | V-90 | V-93 | D-98 | S-103 | Y-104 |
| 11369 | | | | | | | | | |
| US20120015408- | K-3 | R-9 | D-10 | N-27 | K-63 | V-66 | E-71 | A-76 | Y-77 |
| 0002 | | | | | | | | | |
| US20120015408- | K-3 | R-9 | D-10 | N-27 | K-63 | V-66 | E-71 | A-76 | Y-77 |
| 0004 | | | | | | | | | |
| US20120015408- | K-3 | R-9 | D-10 | D-27 | K-63 | V-66 | E-71 | A-76 | Y-77 |
| 0006 | | | | | | | | | |
| US20120015408- | K-3 | R-9 | D-10 | N-27 | K-63 | V-66 | E-71 | A-76 | Y-77 |
| 0008 | | | | | | | | | |
| US20120015408- | K-3 | R-9 | D-10 | N-27 | K-63 | V-66 | E-71 | A-76 | Y-77 |
| 0010 | | | | | | | | | |
| US20120015408- | K-3 | R-9 | D-10 | N-27 | K-63 | V-66 | E-71 | A-76 | Y-77 |
| 0012 | | | | | | | | | |
| US20120015408- | K-3 | R-9 | D-10 | N-27 | K-63 | V-66 | E-71 | A-76 | Y-77 |
| 0014 | | | | | | | | | |
| US20120015408- | K-3 | R-9 | D-10 | N-27 | K-63 | V-66 | E-71 | A-76 | Y-77 |
| 0016 | | | | | | | | | |
| US20120015408- | K-3 | R-9 | D-10 | N-27 | K-63 | V-66 | E-71 | A-76 | Y-77 |
| 0018 | | | | | | | | | |
| US20120015408- | K-3 | R-9 | D-10 | N-27 | K-63 | V-66 | E-71 | A-76 | Y-77 |
| 0020 | | | | | | | | | |
| US20120015408- | K-3 | R-9 | D-10 | N-27 | K-63 | V-66 | E-71 | A-76 | Y-77 |
| 0022 | | | | | | | | | |
| US20120015408- | K-3 | R-9 | D-10 | N-27 | K-63 | V-66 | E-71 | A-76 | Y-77 |
| 0026 | | | | | | | | | |
| US20120015408- | K-3 | R-9 | D-10 | N-27 | K-63 | V-66 | E-71 | A-76 | Y-77 |
| 0028 | | | | | | | | | |
| US20120015408- | K-3 | R-9 | D-10 | D-27 | K-63 | V-66 | E-71 | A-76 | Y-77 |
| 0030 | | | | | | | | | |
| US20120015408- | K-3 | R-9 | D-10 | N-27 | K-63 | V-66 | E-71 | A-76 | Y-77 |
| 0032 | | | | | | | | | |
| US20120015408- | K-3 | R-9 | D-10 | D-27 | K-63 | V-66 | E-71 | A-76 | Y-77 |
| 0034 | | | | | | | | | |
| US20120015408- | K-3 | R-9 | D-10 | N-27 | K-63 | V-66 | E-71 | A-76 | Y-77 |
| 0036 | | | | | | | | | |
| US20120015408- | K-3 | R-9 | D-10 | D-27 | K-63 | V-66 | E-71 | A-76 | Y-77 |
| 0038 | | | | | | | | | |
| US20120015408- | K-3 | R-9 | D-10 | D-27 | K-63 | V-66 | E-71 | A-76 | Y-77 |
| 0040 | | | | | | | | | |
| US20120015408- | K-3 | R-9 | D-10 | N-27 | K-63 | V-66 | E-71 | A-76 | Y-77 |
| 0042 | | | | | | | | | |
| US20120015408- | K-3 | R-9 | D-10 | D-27 | K-63 | V-66 | E-71 | A-76 | Y-77 |
| 0044 | | | | | | | | | |
| US20120015408- | K-3 | R-9 | D-10 | N-27 | K-63 | V-66 | E-71 | A-76 | Y-77 |
| 0046 | | | | | | | | | |
| US20120015408- | K-3 | R-9 | D-10 | D-27 | K-63 | V-66 | E-71 | A-76 | Y-77 |
| 0048 | | | | | | | | | |
| US20120015408- | K-3 | R-9 | D-10 | N-27 | K-63 | V-66 | E-71 | A-76 | Y-77 |
| 0050 | | | | | | | | | |
| US20120015408- | K-3 | R-9 | D-10 | D-27 | K-63 | V-66 | E-71 | A-76 | Y-77 |
| 0052 | | | | | | | | | |
| US20120015408- | K-3 | R-9 | D-10 | D-27 | K-63 | V-66 | E-71 | A-76 | Y-77 |
| 0054 | | | | | | | | | |
| US20120015408- | K-3 | R-9 | D-10 | D-27 | K-63 | V-66 | E-71 | A-76 | Y-77 |
| 0056 | | | | | | | | | |
| US20120015408- | K-3 | R-9 | D-10 | N-27 | K-63 | V-66 | E-71 | A-76 | Y-77 |
| 0058 | | | | | | | | | |
| US20120015408- | K-3 | R-9 | D-10 | D-27 | K-63 | V-66 | E-71 | A-76 | Y-77 |
| 0060 | | | | | | | | | |
| US20120015408- | K-3 | R-9 | D-10 | D-27 | K-63 | V-66 | E-71 | A-76 | Y-77 |
| 0062 | | | | | | | | | |
| US20120015408- | K-3 | R-9 | D-10 | N-27 | K-63 | V-66 | E-71 | A-76 | Y-77 |
| 0064 | | | | | | | | | |
| US20120015408- | K-3 | R-9 | D-10 | D-27 | K-63 | V-66 | E-71 | A-76 | Y-77 |
| 0066 | | | | | | | | | |
| US20120015408- | K-3 | R-9 | D-10 | N-27 | K-63 | V-66 | E-71 | A-76 | Y-77 |
| 0068 | | | | | | | | | |
| US20120015408- | K-3 | R-9 | D-10 | N-27 | K-63 | V-66 | E-71 | A-76 | Y-77 |
| 0070 | | | | | | | | | |
| US20120015408- | K-3 | R-9 | D-10 | D-27 | K-63 | V-66 | E-71 | A-76 | Y-77 |
| 0072 | | | | | | | | | |
| US20120015408- | K-3 | R-9 | D-10 | D-27 | K-63 | V-66 | E-71 | A-76 | Y-77 |
| 0074 | | | | | | | | | |
| US20120015408- | K-3 | R-9 | D-10 | D-27 | K-63 | V-66 | E-71 | A-76 | Y-77 |
| 0076 | | | | | | | | | |
| US20120015408- | K-3 | R-9 | D-10 | D-27 | K-63 | V-66 | E-71 | A-76 | Y-77 |
| 0078 | | | | | | | | | |
| US20120015408- | K-3 | R-9 | D-10 | N-27 | K-63 | V-66 | E-71 | A-76 | Y-77 |
| 0080 | | | | | | | | | |
| US20120015408- | K-3 | R-9 | D-10 | N-27 | K-63 | V-66 | E-71 | A-76 | Y-77 |
| 0082 | | | | | | | | | |
| US20120015408- | K-3 | R-9 | D-10 | D-27 | K-63 | V-66 | E-71 | A-76 | Y-77 |
| 0086 | | | | | | | | | |
| US20120015408- | K-3 | R-9 | D-10 | N-27 | K-63 | V-66 | E-71 | A-76 | Y-77 |
| 0084 | | | | | | | | | |
| US20120015408- | K-3 | R-9 | D-10 | D-27 | K-63 | V-66 | E-71 | A-76 | Y-77 |
| 0088 | | | | | | | | | |
| US20120015408- | K-3 | R-9 | D-10 | D-27 | K-63 | V-66 | E-71 | A-76 | Y-77 |
| 0090 | | | | | | | | | |
| US20120015408- | K-3 | R-9 | D-10 | N-27 | K-63 | V-66 | E-71 | A-76 | Y-77 |
| 0092 | | | | | | | | | |
| US20120015408- | K-3 | R-9 | D-10 | D-27 | K-63 | V-66 | E-71 | A-76 | Y-77 |
| 0094 | | | | | | | | | |
| US20120015408- | K-3 | R-9 | D-10 | D-27 | K-63 | V-66 | E-71 | A-76 | Y-77 |
| 0096 | | | | | | | | | |
| US20120015408- | K-3 | R-9 | D-10 | D-27 | K-63 | V-66 | E-71 | A-76 | Y-77 |
| 0098 | | | | | | | | | |
| US20120015408- | K-3 | R-9 | D-10 | D-27 | K-63 | V-66 | E-71 | A-76 | Y-77 |
| 0100 | | | | | | | | | |
| US20120015408- | K-3 | R-9 | D-10 | D-27 | K-63 | V-66 | E-71 | A-76 | Y-77 |
| 0102 | | | | | | | | | |
| US20120015408- | K-3 | R-9 | D-10 | D-27 | K-63 | V-66 | E-71 | A-76 | Y-77 |
| 0104 | | | | | | | | | |
| US20120015408- | K-3 | R-9 | D-10 | D-27 | K-63 | V-66 | E-71 | A-76 | Y-77 |
| 0106 | | | | | | | | | |
| US20120015408- | K-3 | R-9 | D-10 | D-27 | K-63 | V-66 | E-71 | A-76 | Y-77 |
| 0108 | | | | | | | | | |
| US20120015408- | K-3 | R-9 | D-10 | N-27 | K-63 | V-66 | E-71 | A-76 | Y-77 |
| 0110 | | | | | | | | | |
| US20120015408- | K-3 | R-9 | D-10 | D-27 | K-63 | V-66 | E-71 | A-76 | Y-77 |
| 0112 | | | | | | | | | |
| US20120015408- | K-3 | R-9 | D-10 | D-27 | K-63 | V-66 | E-71 | A-76 | Y-77 |
| 0114 | | | | | | | | | |
| US20120015408- | K-3 | R-9 | D-10 | D-27 | K-63 | V-66 | E-71 | A-76 | Y-77 |
| 0116 | | | | | | | | | |
| US20120015408- | K-3 | R-9 | D-10 | D-27 | K-63 | V-66 | E-71 | A-76 | Y-77 |
| 0118 | | | | | | | | | |
| US20120015408- | K-3 | R-9 | D-10 | D-27 | K-63 | V-66 | E-71 | A-76 | Y-77 |
| 0120 | | | | | | | | | |
| US20120015408- | K-3 | R-9 | D-10 | D-27 | K-63 | V-66 | E-71 | A-76 | Y-77 |
| 0122 | | | | | | | | | |
| US20120015408- | K-3 | R-9 | D-10 | N-27 | K-63 | V-66 | E-71 | A-76 | Y-77 |
| 0124 | | | | | | | | | |
| US8202716-0464 | E-3 | E-9 | G-10 | R-27 | Q-63 | V-66 | E-71 | S-76 | Y-77 |
| US20110262988- | A-2 | K-8 | D-9 | N-26 | E-62 | I-65 | E-70 | S-75 | Y-76 |
| 0002 | | | | | | | | | |
| US20110262988- | S-25 | K-31 | D-32 | N-49 | E-85 | I-88 | E-93 | S-98 | Y-99 |
| 0003 | | | | | | | | | |
| US20110151538- | S-54 | K-60 | D-61 | N-78 | E-114 | I-117 | E-122 | S-127 | Y-128 |
| 0130 | | | | | | | | | |
| US20100003234- | K-2 | E-8 | N-9 | S-26 | V-62 | V-65 | Q-70 | A-75 | Y-76 |
| 0048 | | | | | | | | | |
| US6184018-0012 | S-2 | K-8 | D-9 | N-26 | E-62 | I-65 | E-70 | S-75 | Y-76 |
| US6377893-0063 | — | K-3 | D-4 | N-21 | E-57 | I-60 | E-65 | S-70 | Y-71 |
| ACJ34717 | K-11 | K-17 | D-18 | T-35 | V-71 | V-74 | T-79 | G-84 | Y-85 |
| ADD27066 | K-2 | A-8 | N-9 | S-26 | P-62 | I-65 | E-70 | A-75 | Y-76 |
| ACZ42845 | A-9 | A-15 | D-16 | R-33 | Q-69 | I-72 | R-77 | A-82 | Y-83 |
| ABQ91969 | T-2 | Q-8 | G-9 | R-26 | R-62 | I-65 | S-70 | A-75 | Y-76 |
| ABU56651 | A-2 | D-8 | D-9 | R-26 | R-62 | I-65 | S-70 | A-75 | Y-76 |
| AEY92801 | D-6 | H-12 | D-13 | A-30 | R-66 | I-69 | Q-74 | A-79 | Y-80 |
| CAD55382 | D-20 | D-26 | D-27 | A-44 | H-80 | I-83 | R-88 | A-93 | Y-94 |
| AD073143 | T-2 | K-8 | E-9 | M-26 | R-62 | I-65 | G-70 | H-75 | Y-76 |
| CCA60311 | — | A-18 | G-19 | T-36 | D-72 | V-75 | R-80 | D-85 | Y-86 |
| ADI15206 | P-13 | P-19 | G-20 | H-37 | E-73 | L-76 | S-81 | S-86 | Y-87 |
| ADI12494 | D-6 | H-12 | D-13 | A-30 | R-66 | I-69 | R-74 | A-79 | Y-80 |
| ACU35736 | E-2 | E-8 | G-9 | K-26 | E-62 | V-65 | D-70 | A-75 | Y-76 |
| BAC69512 | D-4 | H-10 | D-11 | A-28 | R-64 | I-67 | Q-72 | A-77 | Y-78 |
| AEN08263 | D-6 | R-12 | D-13 | G-30 | P-66 | I-69 | R-74 | A-79 | Y-80 |
| CCA53915 | D-25 | P-31 | D-32 | A-49 | P-85 | I-88 | G-93 | A-98 | Y-99 |
| CAJ88063 | D-6 | A-12 | D-13 | A-30 | P-66 | I-69 | R-74 | A-79 | Y-80 |
| ABF87202 | — | N-6 | D-7 | S-24 | R-60 | V-63 | W-68 | S-73 | Y-74 |
| BAJ30040 | H-3 | A-9 | N-10 | S-27 | P-63 | L-66 | S-71 | A-76 | Y-77 |
| ACO44852 | T-4 | N-10 | G-11 | S-28 | P-64 | L-67 | E-72 | A-77 | Y-78 |
| AF059750 | — | E-12 | G-13 | H-30 | P-66 | V-69 | D-74 | H-79 | Y-80 |
| BAJ31549 | Q-27 | A-33 | G-34 | D-51 | R-87 | V-90 | E-95 | A-100 | Y-101 |
| ACY97307 | T-18 | Q-24 | D-25 | A-42 | R-78 | V-81 | E-86 | A-91 | Y-92 |
| ACM06095 | S-2 | H-8 | G-9 | G-26 | P-62 | V-65 | E-70 | A-75 | Y-76 |
| AEI64652 | — | N-6 | D-7 | H-24 | R-60 | V-63 | W-68 | S-73 | Y-74 |
| BAG17581 | D-20 | A-26 | D-27 | A-44 | P-80 | I-83 | Q-88 | A-93 | Y-94 |
| AEM83530 | D-6 | H-12 | D-13 | A-30 | R-66 | I-69 | R-74 | A-79 | Y-80 |
| ADG89307 | — | E-16 | G-17 | D-34 | R-70 | V-73 | E-78 | A-83 | Y-84 |
| ABG04991 | — | E-5 | G-6 | G-23 | E-59 | L-62 | R-67 | A-72 | Y-73 |
| ABP54026 | P-17 | P-23 | G-24 | T-41 | D-77 | V-80 | E-85 | S-90 | Y-91 |
| ABV97405 | P-17 | P-23 | G-24 | T-41 | D-77 | V-80 | E-85 | S-90 | Y-91 |
| ADH60167 | — | K-6 | D-7 | N-24 | K-60 | V-63 | E-68 | A-73 | Y-74 |
| ADV80493 | — | K-6 | D-7 | N-24 | K-60 | V-63 | E-68 | A-73 | Y-74 |
| AEM77729 | — | K-6 | E-7 | N-24 | K-60 | V-63 | E-68 | A-73 | Y-74 |
| ABK71329 | — | D-12 | G-13 | T-30 | R-66 | V-69 | D-74 | H-79 | Y-80 |
| ACZ89864 | — | T-20 | G-21 | K-38 | A-74 | V-77 | D-82 | A-87 | Y-88 |
| BAC72965 | — | P-18 | A-19 | R-36 | R-72 | V-75 | E-80 | A-85 | Y-86 |
| ADD25173 | M-1 | K-7 | D-8 | N-25 | K-61 | V-64 | E-69 | A-74 | Y-75 |
| ABF44291 | R-12 | A-18 | G-19 | S-36 | E-72 | L-75 | A-80 | A-85 | Y-86 |
| AEY93261 | G-9 | P-15 | G-16 | R-33 | R-69 | V-72 | D-77 | S-82 | Y-83 |
| 1NP2 | — | E-6 | K-7 | Q-24 | E-60 | I-63 | S-68 | V-73 | Y-74 |
| AEG34643 | — | E-6 | K-7 | Q-24 | E-60 | I-63 | S-68 | A-73 | Y-74 |
| AFR07907 | — | E-9 | D-10 | T-27 | A-63 | I-66 | A-71 | A-76 | Y-77 |
| AEB43702 | A-17 | P-23 | G-24 | A-41 | G-77 | V-80 | E-85 | S-90 | Y-91 |
| BAL92882 | — | D-21 | G-22 | R-39 | A-75 | V-78 | D-83 | A-88 | Y-89 |
| CCA59876 | — | A-17 | D-18 | A-35 | R-71 | V-74 | E-79 | A-84 | Y-85 |
| ACU74192 | — | E-8 | N-9 | A-26 | R-62 | V-65 | K-70 | A-75 | Y-76 |
| ABV96319 | — | D-8 | N-9 | R-26 | A-62 | V-65 | E-70 | A-75 | Y-76 |
| AF053528 | — | K-19 | G-20 | S-37 | R-73 | V-76 | E-81 | A-86 | Y-87 |
| AFH40090 | — | E-6 | K-7 | Q-24 | E-60 | I-63 | S-68 | V-73 | Y-74 |
| ADL49193 | — | E-8 | G-9 | R-26 | A-62 | V-65 | D-70 | A-75 | Y-76 |
| AFE08200 | — | P-6 | D-7 | R-24 | P-60 | I-63 | W-68 | S-73 | Y-74 |
| AAN05441 | — | E-6 | K-7 | Q-24 | E-60 | I-63 | S-68 | A-73 | Y-74 |
| CAJ90043 | — | P-9 | G-10 | R-27 | R-63 | V-66 | D-71 | S-76 | Y-77 |
| AEY89575 | — | P-11 | A-12 | R-29 | R-65 | V-68 | E-73 | A-78 | Y-79 |
| AAF37730 | P-18 | S-24 | D-25 | T-42 | R-78 | V-81 | E-86 | A-91 | Y-92 |
| BAL98072 | S-2 | K-8 | G-9 | Q-26 | R-62 | V-65 | E-70 | A-75 | Y-76 |
| AAN05440 | — | E-6 | K-7 | Q-24 | E-60 | I-63 | S-68 | V-73 | Y-74 |
| AEM77895 | — | K-6 | E-7 | N-24 | K-60 | V-63 | E-68 | A-73 | Y-74 |
| ADB34272 | — | T-17 | G-18 | A-35 | R-71 | V-74 | D-79 | A-84 | Y-85 |
| ABW87307 | — | E-6 | K-7 | Q-24 | E-60 | I-63 | S-68 | V-73 | Y-74 |
| AF322365 1 | — | E-6 | K-7 | Q-24 | E-60 | I-63 | S-68 | V-73 | Y-74 |
| CAC10107 | — | P-15 | A-16 | R-33 | R-69 | V-72 | D-77 | A-82 | Y-83 |
| AEB47478 | — | E-8 | N-9 | R-26 | A-62 | V-65 | E-70 | A-75 | Y-76 |
| ADL45220 | P-17 | S-23 | D-24 | T-41 | P-77 | V-80 | D-85 | S-90 | Y-91 |
| ABK51908 | — | D-20 | R-21 | A-38 | V-74 | V-77 | D-82 | S-87 | Y-88 |
| CAN94460 | P-2 | D-8 | N-9 | N-26 | P-62 | I-65 | S-70 | A-75 | Y-76 |
| AEF18219 | — | K-6 | D-7 | N-24 | K-60 | I-63 | E-68 | A-73 | Y-74 |
| ADD39191 | — | A-29 | G-30 | R-47 | R-83 | V-86 | E-91 | A-96 | Y-97 |
| ABP52811 | — | E-8 | N-9 | H-26 | A-62 | V-65 | G-70 | T-75 | Y-76 |
| AEV88819 | — | D-36 | G-37 | R-54 | V-90 | V-93 | D-98 | S-103 | Y-104 |
| AEY89570 | — | T-20 | G-21 | A-38 | R-74 | V-77 | E-82 | A-87 | Y-88 |
| ADD01635 | — | K-6 | D-7 | D-24 | K-60 | V-63 | E-68 | A-73 | Y-74 |
| CAB95278 | — | P-9 | G-10 | T-27 | P-63 | V-66 | D-71 | S-76 | Y-77 |
| AEN13042 | — | P-9 | G-10 | E-27 | P-63 | V-66 | D-71 | S-76 | Y-77 |
| ACY97750 | — | P-12 | G-13 | D-30 | A-66 | V-69 | E-74 | A-79 | Y-80 |
| ADI10010 | — | P-7 | G-8 | Q-25 | R-61 | V-64 | D-69 | A-74 | Y-75 |
| CCB72805 | — | T-15 | G-16 | R-33 | A-69 | V-72 | G-77 | A-82 | Y-83 |
| ACY14034 | — | A-20 | D-21 | N-38 | P-74 | V-77 | S-82 | T-87 | Y-88 |
| AAZ55664 | — | A-11 | G-12 | H-29 | P-65 | V-68 | R-73 | A-78 | Y-79 |
| ADW02698 | — | P-9 | G-10 | E-27 | P-63 | V-66 | D-71 | S-76 | Y-77 |
| ABC33525 | — | K-7 | D-8 | N-25 | Q-61 | V-64 | G-69 | A-74 | Y-75 |
| ADB34282 | E-3 | Q-9 | D-10 | A-27 | P-63 | L-66 | Q-71 | A-76 | Y-77 |
| AAM23648 | E-3 | K-9 | D-10 | N-27 | K-63 | V-66 | E-71 | A-76 | Y-77 |
| CCH86028 | — | P-18 | G-19 | D-36 | P-72 | V-75 | D-80 | A-85 | Y-86 |
| ADV67544 | H-9 | A-15 | D-16 | N-33 | P-69 | L-72 | D-77 | A-82 | Y-83 |
| ACZ89862 | — | E-21 | G-22 | R-39 | P-75 | V-78 | D-83 | A-88 | Y-89 |
| ADH66252 | — | E-9 | G-10 | T-27 | A-63 | V-66 | Q-71 | A-76 | Y-77 |
| ADV80605 | — | R-6 | D-7 | N-24 | K-60 | V-63 | E-68 | A-73 | Y-74 |
| ADW05507 | — | T-20 | G-21 | A-38 | R-74 | V-77 | Q-82 | A-87 | Y-88 |
| BAA86923 | — | E-6 | K-7 | Q-24 | E-60 | I-63 | S-68 | A-73 | Y-74 |
| AAN05438 | — | E-6 | K-7 | Q-24 | E-60 | I-63 | S-68 | A-73 | Y-74 |
| CAC16438 | — | K-20 | G-21 | A-38 | S-74 | V-77 | E-82 | A-87 | Y-88 |
| CAJ89567 | — | K-20 | G-21 | A-38 | R-74 | V-77 | E-82 | A-87 | Y-88 |
| AFD27167 | T-20 | Q-26 | G-27 | S-44 | E-80 | L-83 | S-88 | A-93 | Y-94 |
| BAG20044 | — | T-27 | G-28 | T-45 | P-81 | V-84 | E-89 | D-94 | Y-95 |
| ACU71435 | — | R-7 | D-8 | G-25 | P-61 | V-64 | S-69 | T-74 | Y-75 |
| ADG87563 | — | D-43 | G-44 | A-61 | A-97 | V-100 | G-105 | A-110 | Y-111 |
| ABI35984 | — | E-6 | K-7 | Q-24 | E-60 | I-63 | S-68 | A-73 | Y-74 |
| CAB42553 | — | E-6 | K-7 | Q-24 | E-60 | I-63 | S-68 | A-73 | Y-74 |
| 1UG6 | — | E-6 | K-7 | Q-24 | E-60 | I-63 | S-68 | A-73 | Y-74 |
| CAA91220 | K-3 | R-9 | D-10 | N-27 | K-63 | V-66 | E-71 | A-76 | Y-77 |
| AFK85369 | — | K-6 | G-7 | N-24 | K-60 | V-63 | E-68 | A-73 | Y-74 |
| AEN10177 | — | T-21 | G-22 | A-39 | R-75 | V-78 | R-83 | A-88 | Y-89 |
| AEM87460 | — | P-7 | G-8 | Q-25 | R-61 | V-64 | E-69 | A-74 | Y-75 |
| AAZ81839 | E-3 | E-9 | G-10 | R-27 | Q-63 | V-66 | E-71 | S-76 | Y-77 |
| ADU50085 | — | E-31 | G-32 | H-49 | A-85 | V-88 | R-93 | S-98 | Y-99 |
| AEK47062 | — | P-20 | D-21 | S-38 | P-74 | I-77 | E-82 | T-87 | Y-88 |
| ABD68852 | K-13 | L-19 | D-20 | D-37 | P-73 | L-76 | S-81 | A-86 | Y-87 |
| ACL38401 | — | E-10 | D-11 | H-28 | R-64 | V-67 | R-72 | A-77 | Y-78 |
| ABS05424 | — | P-22 | G-23 | A-40 | R-76 | I-79 | R-84 | S-89 | Y-90 |
| AEK43773 | — | P-4 | G-5 | T-22 | S-58 | V-61 | R-66 | A-71 | Y-72 |
| AEV87561 | — | M-1 | T-2 | A-19 | P-51 | V-54 | S-59 | A-64 | Y-65 |
| ACZ86244 | P-11 | T-17 | G-18 | S-35 | R-71 | V-74 | D-79 | A-84 | Y-85 |
| AEB46173 | — | P-8 | G-9 | K-26 | A-62 | L-65 | A-70 | A-75 | Y-76 |
| BAG21567 | — | T-22 | G-23 | A-40 | R-76 | V-79 | R-84 | A-89 | Y-90 |
| ABX05041 | T-2 | S-8 | D-9 | F-26 | R-62 | V-65 | R-70 | A-75 | Y-76 |
| AFR09943 | — | P-25 | D-26 | R-43 | E-79 | V-82 | E-87 | T-92 | Y-93 |
| ACZ89285 | — | A-16 | G-17 | D-34 | P-70 | V-73 | G-78 | A-83 | Y-84 |
| ACV58907 | — | E-6 | G-7 | R-24 | Q-60 | V-63 | E-68 | S-73 | Y-74 |
| ADG89462 | — | — | M-1 | R-18 | P-54 | V-57 | E-62 | A-67 | Y-68 |
| ADU09756 | — | A-25 | G-26 | D-43 | R-79 | V-82 | G-87 | A-92 | Y-93 |
| ACZ90607 | — | — | M-1 | A-18 | P-54 | V-57 | G-62 | S-67 | Y-68 |
| ADG73989 | — | A-14 | D-15 | A-32 | P-68 | V-71 | D-76 | A-81 | Y-82 |
| AEG45154 | G-34 | D-40 | G-41 | S-58 | R-94 | V-97 | R-102 | T-107 | Y-108 |
| AEJ43907 | — | D-6 | A-7 | R-24 | E-60 | V-63 | E-68 | S-73 | Y-74 |
| ACZ20790 | — | E-21 | G-22 | R-39 | A-75 | T-78 | D-83 | A-88 | Y-89 |
| CCB77455 | P-4 | S-10 | D-11 | T-28 | P-64 | V-67 | G-72 | A-77 | Y-78 |
| ADL46625 | — | P-22 | T-23 | A-40 | P-76 | V-79 | D-84 | T-89 | Y-90 |
| ADU10772 | — | P-22 | T-23 | A-40 | P-76 | V-79 | D-84 | T-89 | Y-90 |
| ADD45899 | — | P-10 | D-11 | T-28 | D-64 | V-67 | E-72 | A-77 | Y-78 |
| CCA53920 | — | P-21 | H-22 | A-39 | P-75 | V-78 | S-83 | T-88 | Y-89 |
| BAJ28512 | — | P-21 | G-22 | A-39 | R-75 | V-78 | D-83 | S-88 | Y-89 |
| ACL70277 | A-2 | E-8 | D-9 | N-26 | R-62 | I-65 | E-70 | S-75 | Y-76 |
| CBG72797 | — | P-15 | A-16 | R-33 | R-69 | V-72 | D-77 | A-82 | Y-83 |
| AEW05616 | H-3 | A-9 | D-10 | N-27 | A-63 | I-66 | D-71 | S-76 | Y-77 |
| AEB46623 | — | A-6 | G-7 | G-24 | A-60 | I-63 | E-68 | A-73 | Y-74 |
| 1GNX | — | E-20 | G-21 | A-38 | R-74 | V-77 | E-82 | A-87 | Y-88 |
| ACV76621 | — | E-10 | D-11 | T-28 | S-64 | L-67 | G-72 | A-77 | Y-78 |
| ACZ31628 | G-35 | D-41 | G-42 | R-59 | R-95 | V-98 | S-103 | T-108 | Y-109 |
| ADH67953 | — | A-8 | S-9 | H-26 | G-62 | V-65 | D-70 | L-75 | Y-76 |
| ADB34290 | — | A-21 | G-22 | D-39 | P-75 | V-78 | D-83 | T-88 | Y-89 |
| ADU09106 | P-29 | A-35 | G-36 | T-53 | A-89 | T-92 | G-97 | A-102 | Y-103 |
| CAA82733 | — | E-20 | G-21 | A-38 | R-74 | V-77 | E-82 | A-87 | Y-88 |
| CBG67455 | — | R-37 | G-38 | A-55 | N-91 | V-94 | E-99 | A-104 | Y-105 |
| CAN00920 | — | P-27 | G-28 | D-45 | D-81 | L-84 | A-89 | A-94 | Y-95 |
| ADX71280 | — | Q-10 | G-11 | H-28 | K-64 | V-67 | S-72 | A-77 | Y-78 |
| ACQ81085 | V-18 | A-24 | D-25 | W-42 | A-78 | V-81 | E-86 | A-91 | Y-92 |
| 3AHX | E-2 | K-8 | D-9 | K-26 | K-62 | V-65 | S-70 | S-75 | Y-76 |
| ACU35632 | — | P-19 | S-20 | A-37 | P-73 | V-76 | S-81 | T-86 | Y-87 |
| AEV86556 | — | D-12 | G-13 | A-30 | R-63 | V-66 | D-71 | A-76 | Y-77 |
| AEG45006 | — | T-19 | D-20 | T-37 | P-73 | V-76 | R-81 | A-86 | Y-87 |
| BAG18801 | — | A-21 | D-22 | A-39 | P-75 | V-78 | G-83 | T-88 | Y-89 |
| ADI03707 | R-13 | P-19 | G-20 | D-37 | R-73 | V-76 | R-81 | S-86 | Y-87 |
| CAQ00266 | — | P-27 | G-28 | D-45 | E-81 | L-84 | A-89 | A-94 | Y-95 |
| ADD43929 | A-3 | D-9 | G-10 | D-27 | G-63 | I-66 | R-71 | V-76 | Y-77 |
| ADJ49823 | — | K-5 | G-6 | T-23 | P-59 | I-62 | D-67 | A-72 | Y-73 |
| ABX05062 | V-4 | A-10 | D-11 | H-28 | P-64 | L-67 | E-72 | S-77 | Y-78 |
| ADL48215 | P-5 | A-11 | G-12 | T-29 | A-65 | T-68 | G-73 | A-78 | Y-79 |
| ACU70272 | P-6 | A-12 | S-13 | A-30 | A-66 | T-69 | D-74 | A-79 | Y-80 |
| ADG88606 | P-11 | P-17 | E-18 | S-35 | R-71 | V-74 | E-79 | A-84 | Y-85 |
| AEW47954 | T-3 | S-9 | N-10 | H-27 | K-63 | I-66 | Q-71 | A-76 | Y-77 |
| ACD20223 | D-28 | K-34 | S-35 | N-52 | Q-88 | V-91 | G-96 | A-101 | Y-102 |
| AFC28171 | N-2 | S-8 | D-9 | S-26 | P-62 | I-65 | E-70 | S-75 | Y-76 |
| ADW03239 | — | A-20 | G-21 | S-38 | P-74 | V-77 | G-82 | T-87 | Y-88 |
| CBT74727 | — | K-13 | E-14 | H-31 | R-67 | V-70 | E-75 | S-80 | Y-81 |
| CAM04686 | — | P-11 | G-12 | K-29 | A-65 | V-68 | E-73 | A-78 | Y-79 |
| ACZ20966 | — | A-13 | D-14 | T-31 | A-67 | V-70 | D-75 | A-80 | Y-81 |
| AEV38153 | N-17 | K-23 | D-24 | N-41 | E-77 | V-80 | E-85 | A-90 | Y-91 |
| BAJ26623 | R-4 | A-9 | G-10 | D-27 | P-63 | V-66 | D-71 | G-76 | Y-77 |
| BAL91665 | — | M-1 | T-2 | F-19 | P-51 | V-54 | S-59 | A-64 | Y-65 |
| ACV09397 | — | A-11 | D-12 | R-29 | S-65 | I-68 | A-73 | A-78 | Y-79 |
| ADG20157 | — | E-8 | D-9 | H-26 | E-62 | L-65 | E-70 | A-75 | Y-76 |
| ADC61565 | — | D-7 | D-8 | L-25 | L-61 | V-64 | E-69 | A-74 | Y-75 |
| ACM66669 | — | K-13 | E-14 | H-31 | R-67 | V-70 | E-75 | S-80 | Y-81 |
| ABD68843 | — | K-15 | N-16 | E-33 | A-69 | L-72 | G-77 | A-82 | Y-83 |
| ABS61373 | K-3 | K-9 | D-10 | K-27 | K-63 | V-66 | E-71 | A-76 | Y-77 |
| CBA30283 | P-16 | S-22 | D-23 | A-40 | E-76 | L-79 | S-84 | S-89 | Y-90 |
| AAA22266 | S-2 | S-8 | D-9 | N-26 | E-62 | V-65 | D-70 | V-75 | Y-76 |
| CAA42814 | S-2 | K-8 | D-9 | N-26 | E-62 | I-65 | E-70 | S-75 | Y-76 |
| ABN51453 | S-25 | K-31 | D-32 | N-49 | E-85 | I-88 | E-93 | S-98 | Y-99 |
| ACZ00292 | — | A-5 | D-6 | T-23 | P-59 | L-62 | D-67 | A-72 | Y-73 |
| AEI12946 | — | A-14 | D-15 | Q-32 | P-68 | V-71 | E-76 | A-81 | Y-82 |
| ABS15474 | S-17 | K-23 | D-24 | Y-41 | R-77 | I-80 | A-85 | A-90 | Y-91 |
| ADL51094 | E-2 | K-8 | D-9 | K-26 | K-62 | V-65 | S-70 | S-75 | Y-76 |
| AFG34202 | R-3 | K-9 | D-10 | N-27 | K-63 | I-66 | E-71 | A-76 | Y-77 |
| AAN60220 | R-3 | K-9 | D-10 | N-27 | K-63 | I-66 | E-71 | A-76 | Y-77 |

| Corresp. | Sequence Name | | | | | | | |
|---|---|---|---|---|---|---|---|---|
| Sequence Pos. | E105 | Q139 | Q142 | D154 | V167 | N175 | V203 | I216 |
| SEQ ID NO. | E-105 | Q-139 | Q-142 | D-154 | V-167 | N-175 | V-203 | I-216 |
| 379/380 | | | | | | | | |
| SEQ ID NO. 378 | A-109 | Y-143 | A-146 | D-158 | C-171 | N-179 | T-207 | V-219 |
| US8101393-0094 | E-105 | H-139 | Q-142 | D-154 | V-167 | N-175 | V-203 | I-216 |
| US8101393-0388 | E-105 | Q-139 | Q-142 | D-154 | V-167 | N-175 | V-203 | I-216 |
| US8101393-0172 | A-109 | Y-143 | A-146 | D-158 | C-171 | N-179 | T-207 | V-219 |
| JP2011205992-0018 | A-107 | T-141 | A-144 | D-156 | C-169 | L-177 | V-205 | I-218 |
| JP2011205992-0023 | A-107 | Y-141 | A-144 | D-156 | C-169 | A-177 | V-205 | V-217 |
| JP2011205992-0022 | A-107 | D-141 | A-144 | D-156 | C-169 | I-177 | V-207 | V-219 |
| US20110214199- | G-109 | E-143 | A-146 | D-158 | C-171 | E-179 | A-207 | V-219 |
| 58656 | | | | | | | | |
| US20110214199- | D-125 | E-159 | A-162 | D-174 | C-187 | E-195 | M-223 | V-235 |
| 62406 | | | | | | | | |
| US20110214199- | G-105 | S-139 | V-142 | D-154 | C-167 | N-175 | V-203 | V-215 |
| 47919 | | | | | | | | |
| US20110214199- | G-104 | E-138 | A-141 | G-153 | V-166 | L-174 | L-202 | L-214 |
| 60662 | | | | | | | | |
| US20110214199- | E-111 | Q-145 | A-148 | D-160 | C-173 | A-181 | V-209 | L-221 |
| 36660 | | | | | | | | |
| US20110214199- | E-117 | Y-151 | A-154 | D-166 | C-179 | S-187 | T-215 | V-228 |
| 17908 | | | | | | | | |
| US20110214199- | R-105 | F-139 | A-142 | D-154 | C-167 | T-175 | V-203 | V-214 |
| 25308 | | | | | | | | |
| US20110214199- | R-105 | F-139 | A-142 | D-154 | C-167 | T-175 | V-203 | V-214 |
| 64004 | | | | | | | | |
| US20110214199- | C-123 | K-157 | A-160 | D-172 | C-185 | S-193 | A-221 | I-237 |
| 25023 | | | | | | | | |
| US20110214199- | R-105 | F-139 | A-142 | D-154 | C-167 | T-175 | V-203 | V-214 |
| 52644 | | | | | | | | |
| US20110214199- | E-114 | Y-148 | A-151 | D-163 | C-176 | S-184 | T-212 | V-225 |
| 13862 | | | | | | | | |
| US20110214199- | E-107 | K-142 | V-145 | D-157 | C-170 | I-178 | V-206 | I-219 |
| 29446 | | | | | | | | |
| US20110214199- | R-105 | F-139 | A-142 | D-154 | C-167 | T-175 | V-203 | V-214 |
| 5988 | | | | | | | | |
| US20110214199- | R-105 | F-139 | A-142 | D-154 | C-167 | T-175 | V-203 | V-214 |
| 23246 | | | | | | | | |
| US20110214199- | E-107 | D-141 | V-144 | D-156 | V-169 | L-177 | V-205 | I-217 |
| 12000 | | | | | | | | |
| US20110214199- | E-107 | L-141 | R-144 | D-156 | C-169 | T-177 | V-205 | V-217 |
| 56949 | | | | | | | | |
| US8101393-0272 | R-117 | Y-151 | A-154 | D-166 | C-179 | N-187 | A-215 | V-228 |
| US8101393-0014 | E-111 | E-145 | V-148 | D-160 | C-173 | I-181 | V-209 | V-221 |
| US8101393-0398 | E-122 | A-156 | A-159 | D-171 | C-184 | S-192 | V-220 | L-232 |
| US8101393-0492 | E-122 | A-156 | A-159 | D-171 | C-184 | S-192 | V-220 | L-232 |
| US8101393-0400 | E-122 | A-156 | A-159 | D-171 | C-184 | S-192 | V-220 | L-232 |
| US8101393-0266 | E-116 | E-150 | A-153 | D-165 | C-178 | S-186 | V-214 | L-227 |
| US8101393-0366 | A-103 | Y-137 | A-140 | D-152 | C-165 | T-173 | V-201 | V-212 |
| US8101393-0342 | G-107 | K-141 | V-144 | D-156 | C-169 | I-177 | V-205 | I-217 |
| US8101393-0356 | A-107 | E-141 | T-144 | D-156 | C-169 | L-177 | V-205 | V-217 |
| US8101393-0320 | L-107 | D-141 | T-144 | D-156 | V-169 | L-177 | V-205 | I-217 |
| US7314974-19451 | G-103 | S-137 | V-140 | D-152 | C-165 | N-173 | V-201 | V-213 |
| US7314974-8251 | C-98 | K-132 | A-135 | D-147 | C-160 | S-168 | A-196 | I-212 |
| US7630836-9340 | G-109 | E-143 | A-146 | D-158 | C-171 | E-179 | A-207 | V-219 |
| US7630836-12787 | E-117 | Y-151 | A-154 | D-166 | C-179 | S-187 | T-215 | V-228 |
| US20090220480- | R-105 | G-139 | V-142 | D-154 | C-167 | N-175 | V-203 | V-215 |
| 0018 | | | | | | | | |
| US20120034253- | R-105 | F-139 | A-142 | D-154 | C-167 | T-175 | V-203 | V-214 |
| 0080 | | | | | | | | |
| US20120034253- | R-105 | F-139 | A-142 | D-154 | C-167 | T-175 | V-203 | V-214 |
| 0077 | | | | | | | | |
| US20120034253- | R-105 | F-139 | A-142 | D-154 | C-167 | T-175 | V-203 | V-214 |
| 0078 | | | | | | | | |
| WO2012016960- | E-135 | E-169 | G-172 | D-184 | C-197 | S-205 | A-233 | V-244 |
| 11369 | | | | | | | | |
| US20120015408- | E-107 | K-142 | V-145 | D-157 | C-170 | I-178 | V-206 | I-219 |
| 0002 | | | | | | | | |
| US20120015408- | E-107 | K-142 | V-145 | D-157 | C-170 | I-178 | V-206 | I-219 |
| 0004 | | | | | | | | |
| US20120015408- | E-107 | K-142 | V-145 | D-157 | C-170 | I-178 | V-206 | I-219 |
| 0006 | | | | | | | | |
| US20120015408- | E-107 | K-142 | V-145 | D-157 | C-170 | I-178 | V-206 | I-219 |
| 0008 | | | | | | | | |
| US20120015408- | E-107 | K-142 | V-145 | D-157 | C-170 | I-178 | V-206 | I-219 |
| 0010 | | | | | | | | |
| US20120015408- | E-107 | K-142 | V-145 | D-157 | C-170 | I-178 | V-206 | I-219 |
| 0012 | | | | | | | | |
| US20120015408- | E-107 | K-142 | V-145 | D-157 | C-170 | I-178 | V-206 | I-219 |
| 0014 | | | | | | | | |
| US20120015408- | E-107 | K-142 | V-145 | D-157 | C-170 | I-178 | V-206 | I-219 |
| 0016 | | | | | | | | |
| US20120015408- | E-107 | K-142 | V-145 | D-157 | C-170 | I-178 | V-206 | I-219 |
| 0018 | | | | | | | | |
| US20120015408- | E-107 | K-142 | V-145 | D-157 | C-170 | I-178 | V-206 | I-219 |
| 0020 | | | | | | | | |
| US20120015408- | E-107 | K-142 | V-145 | D-157 | C-170 | I-178 | V-206 | I-219 |
| 0022 | | | | | | | | |
| US20120015408- | E-107 | K-142 | V-145 | D-157 | C-170 | I-178 | V-206 | I-219 |
| 0026 | | | | | | | | |
| US20120015408- | E-107 | K-142 | V-145 | D-157 | C-170 | I-178 | V-206 | I-219 |
| 0028 | | | | | | | | |
| US20120015408- | E-107 | K-142 | V-145 | D-157 | C-170 | I-178 | V-206 | I-219 |
| 0030 | | | | | | | | |
| US20120015408- | E-107 | K-142 | V-145 | D-157 | C-170 | I-178 | V-206 | I-219 |
| 0032 | | | | | | | | |
| US20120015408- | E-107 | K-142 | V-145 | D-157 | C-170 | I-178 | V-206 | I-219 |
| 0034 | | | | | | | | |
| US20120015408- | E-107 | K-142 | V-145 | D-157 | C-170 | I-178 | V-206 | I-219 |
| 0036 | | | | | | | | |
| US20120015408- | E-107 | K-142 | V-145 | D-157 | C-170 | I-178 | V-206 | I-219 |
| 0038 | | | | | | | | |
| US20120015408- | E-107 | K-142 | V-145 | D-157 | C-170 | I-178 | V-206 | I-219 |
| 0040 | | | | | | | | |
| US20120015408- | E-107 | E-142 | V-145 | D-157 | C-170 | I-178 | V-206 | I-219 |
| 0042 | | | | | | | | |
| US20120015408- | E-107 | K-142 | V-145 | D-157 | C-170 | I-178 | V-206 | I-219 |
| 0044 | | | | | | | | |
| US20120015408- | E-107 | K-142 | V-145 | D-157 | C-170 | I-178 | V-206 | I-219 |
| 0046 | | | | | | | | |
| US20120015408- | E-107 | K-142 | V-145 | D-157 | C-170 | I-178 | V-206 | I-219 |
| 0048 | | | | | | | | |
| US20120015408- | E-107 | K-142 | V-145 | D-157 | C-170 | I-178 | V-206 | I-219 |
| 0050 | | | | | | | | |
| US20120015408- | E-107 | K-142 | V-145 | D-157 | C-170 | I-178 | V-206 | I-219 |
| 0052 | | | | | | | | |
| US20120015408- | E-107 | K-142 | V-145 | D-157 | C-170 | I-178 | V-206 | I-219 |
| 0054 | | | | | | | | |
| US20120015408- | E-107 | K-142 | V-145 | D-157 | C-170 | I-178 | V-206 | I-219 |
| 0056 | | | | | | | | |
| US20120015408- | E-107 | K-142 | V-145 | D-157 | C-170 | I-178 | V-206 | I-219 |
| 0058 | | | | | | | | |
| US20120015408- | E-107 | K-142 | V-145 | D-157 | C-170 | I-178 | V-206 | I-219 |
| 0060 | | | | | | | | |
| US20120015408- | E-107 | K-142 | V-145 | D-157 | C-170 | I-178 | V-206 | I-219 |
| 0062 | | | | | | | | |
| US20120015408- | E-107 | K-142 | V-145 | D-157 | C-170 | I-178 | V-206 | I-219 |
| 0064 | | | | | | | | |
| US20120015408- | E-107 | K-142 | V-145 | D-157 | C-170 | I-178 | V-206 | I-219 |
| 0066 | | | | | | | | |
| US20120015408- | E-107 | K-142 | V-145 | D-157 | C-170 | I-178 | V-206 | I-219 |
| 0068 | | | | | | | | |
| US20120015408- | E-107 | K-142 | V-145 | D-157 | C-170 | I-178 | V-206 | I-219 |
| 0070 | | | | | | | | |
| US20120015408- | E-107 | K-142 | V-145 | D-157 | C-170 | I-178 | V-206 | I-219 |
| 0072 | | | | | | | | |
| US20120015408- | E-107 | K-142 | V-145 | D-157 | C-170 | I-178 | V-206 | I-219 |
| 0074 | | | | | | | | |
| US20120015408- | E-107 | K-142 | V-145 | D-157 | C-170 | I-178 | V-206 | I-219 |
| 0076 | | | | | | | | |
| US20120015408- | E-107 | K-142 | V-145 | D-157 | C-170 | I-178 | V-206 | I-219 |
| 0078 | | | | | | | | |
| US20120015408- | E-107 | K-142 | V-145 | D-157 | C-170 | I-178 | V-206 | I-219 |
| 0080 | | | | | | | | |
| US20120015408- | E-107 | K-142 | V-145 | D-157 | C-170 | I-178 | V-206 | I-219 |
| 0082 | | | | | | | | |
| US20120015408- | E-107 | K-142 | V-145 | D-157 | C-170 | I-178 | V-206 | I-219 |
| 0086 | | | | | | | | |
| US20120015408- | E-107 | K-142 | V-145 | D-157 | C-170 | I-178 | V-206 | I-219 |
| 0084 | | | | | | | | |
| US20120015408- | E-107 | K-142 | V-145 | D-157 | C-170 | I-178 | V-206 | I-219 |
| 0088 | | | | | | | | |
| US20120015408- | E-107 | K-142 | V-145 | D-157 | C-170 | I-178 | V-206 | I-219 |
| 0090 | | | | | | | | |
| US20120015408- | E-107 | K-142 | V-145 | D-157 | C-170 | I-178 | V-206 | I-219 |
| 0092 | | | | | | | | |
| US20120015408- | E-107 | K-142 | V-145 | D-157 | C-170 | I-178 | V-206 | I-219 |
| 0094 | | | | | | | | |
| US20120015408- | E-107 | K-142 | V-145 | D-157 | C-170 | I-178 | V-206 | I-219 |
| 0096 | | | | | | | | |
| US20120015408- | E-107 | K-142 | V-145 | D-157 | C-170 | I-178 | V-206 | I-219 |
| 0098 | | | | | | | | |
| US20120015408- | E-107 | K-142 | V-145 | D-157 | C-170 | I-178 | V-206 | I-219 |
| 0100 | | | | | | | | |
| US20120015408- | E-107 | K-142 | V-145 | D-157 | C-170 | I-178 | V-206 | I-219 |
| 0102 | | | | | | | | |
| US20120015408- | E-107 | K-142 | V-145 | D-157 | C-170 | I-178 | V-206 | I-219 |
| 0104 | | | | | | | | |
| US20120015408- | E-107 | K-142 | V-145 | D-157 | C-170 | I-178 | V-206 | I-219 |
| 0106 | | | | | | | | |
| US20120015408- | E-107 | K-142 | V-145 | D-157 | C-170 | I-178 | V-206 | I-219 |
| 0108 | | | | | | | | |
| US20120015408- | E-107 | K-142 | V-145 | D-157 | C-170 | I-178 | V-206 | I-219 |
| 0110 | | | | | | | | |
| US20120015408- | E-107 | K-142 | V-145 | D-157 | C-170 | I-178 | V-206 | I-219 |
| 0112 | | | | | | | | |
| US20120015408- | E-107 | K-142 | V-145 | D-157 | C-170 | I-178 | V-206 | I-219 |
| 0114 | | | | | | | | |
| US20120015408- | E-107 | K-142 | V-145 | D-157 | C-170 | I-178 | V-206 | I-219 |
| 0116 | | | | | | | | |
| US20120015408- | E-107 | K-142 | V-145 | D-157 | C-170 | I-178 | V-206 | I-219 |
| 0118 | | | | | | | | |
| US20120015408- | E-107 | K-142 | V-145 | D-157 | C-170 | I-178 | V-206 | I-219 |
| 0120 | | | | | | | | |
| US20120015408- | E-107 | K-142 | V-145 | D-157 | C-170 | I-178 | V-206 | I-219 |
| 0122 | | | | | | | | |
| US20120015408- | E-107 | K-142 | V-145 | D-157 | C-170 | I-178 | V-206 | I-219 |
| 0124 | | | | | | | | |
| US8202716-0464 | E-107 | S-141 | L-144 | D-156 | C-169 | I-177 | V-205 | I-217 |
| US20110262988- | L-107 | D-141 | T-144 | D-156 | V-169 | L-177 | V-205 | I-217 |
| 0002 | | | | | | | | |
| US20110262988- | L-130 | D-164 | T-167 | D-179 | V-192 | L-200 | V-228 | I-240 |
| 0003 | | | | | | | | |
| US20110151538- | L-159 | D-193 | T-196 | D-208 | V-221 | L-229 | V-257 | I-269 |
| 0130 | | | | | | | | |
| US20100003234- | Q-107 | K-141 | S-144 | L-156 | V-169 | F-177 | V-205 | V-218 |
| 0048 | | | | | | | | |
| US6184018-0012 | L-107 | D-141 | T-144 | D-156 | V-169 | L-177 | V-205 | I-217 |
| US6377893-0063 | L-102 | D-136 | T-139 | D-151 | V-164 | L-172 | V-200 | I-212 |
| ACJ34717 | E-116 | Y-150 | Q-153 | D-165 | V-178 | N-186 | V-214 | I-227 |
| ADD27066 | A-107 | Y-141 | A-144 | D-156 | C-169 | A-177 | V-205 | V-217 |
| ACZ42845 | G-114 | K-148 | A-151 | D-163 | V-176 | T-184 | V-212 | V-224 |
| ABQ91969 | G-107 | S-141 | A-144 | D-156 | C-169 | T-177 | L-204 | A-216 |
| ABU56651 | G-107 | A-141 | A-144 | D-156 | C-169 | T-177 | L-204 | A-216 |
| AEY92801 | A-111 | L-145 | A-148 | D-160 | C-173 | E-181 | T-209 | V-221 |
| CAD55382 | D-125 | E-159 | A-162 | D-174 | C-187 | E-195 | M-223 | V-235 |
| AD073143 | A-107 | E-141 | V-144 | D-156 | C-169 | M-177 | V-205 | V-217 |
| CCA60311 | R-117 | Y-151 | A-154 | D-166 | C-179 | N-187 | A-215 | V-228 |
| ADI15206 | G-118 | Y-152 | A-155 | D-167 | C-180 | T-188 | L-216 | H-228 |
| ADI12494 | G-111 | E-145 | A-148 | D-160 | C-173 | E-181 | T-209 | V-221 |
| ACU35736 | T-106 | H-140 | A-143 | D-155 | C-168 | G-176 | T-204 | V-216 |
| BAC69512 | G-109 | E-143 | A-146 | D-158 | C-171 | E-179 | A-207 | V-219 |
| AEN08263 | G-111 | E-145 | A-148 | D-160 | C-173 | E-181 | T-209 | I-221 |
| CCA53915 | G-130 | E-169 | A-172 | D-184 | C-197 | E-205 | T-233 | V-245 |
| CAJ88063 | G-111 | E-145 | A-148 | D-160 | C-173 | E-181 | T-209 | I-221 |
| ABF87202 | G-105 | S-139 | V-142 | D-154 | C-167 | N-175 | V-203 | V-215 |
| BAJ30040 | G-108 | E-142 | A-145 | D-157 | C-170 | E-178 | V-206 | I-218 |
| ACO44852 | G-109 | H-143 | A-146 | D-158 | C-171 | I-179 | V-207 | L-219 |
| AF059750 | S-111 | E-145 | A-148 | D-160 | C-173 | S-181 | V-209 | L-220 |
| BAJ31549 | E-132 | Y-166 | A-169 | D-181 | C-194 | N-202 | T-230 | V-243 |
| ACY97307 | A-123 | L-157 | A-160 | D-172 | C-185 | S-193 | L-221 | I-233 |
| ACM06095 | A-107 | E-141 | A-144 | D-156 | V-169 | L-177 | A-205 | V-217 |
| AEI64652 | G-105 | N-139 | V-142 | D-154 | C-167 | N-175 | V-203 | V-215 |
| BAG17581 | G-125 | E-159 | A-162 | D-174 | C-187 | E-195 | V-223 | I-235 |
| AEM83530 | G-111 | E-145 | A-148 | D-160 | C-173 | E-181 | A-209 | I-221 |
| ADG89307 | E-115 | E-149 | A-152 | D-164 | C-177 | E-185 | T-213 | V-224 |
| ABG04991 | G-104 | E-138 | A-141 | G-153 | V-166 | L-174 | L-202 | L-214 |
| ABP54026 | Q-122 | A-156 | A-159 | D-171 | C-184 | S-192 | V-220 | I-233 |
| ABV97405 | Q-122 | A-156 | A-159 | D-171 | C-184 | S-192 | V-220 | V-233 |
| ADH60167 | E-104 | K-139 | A-142 | D-154 | C-167 | I-175 | V-203 | V-216 |
| ADV80493 | E-104 | K-139 | V-142 | D-154 | C-167 | I-175 | V-203 | I-216 |
| AEM77729 | E-104 | K-139 | V-142 | D-154 | C-167 | I-175 | V-203 | I-216 |
| ABK71329 | E-111 | Q-145 | A-148 | D-160 | C-173 | A-181 | V-209 | L-221 |
| ACZ89864 | K-119 | H-153 | A-156 | D-168 | V-181 | N-189 | T-217 | I-228 |
| BAC72965 | E-117 | Y-151 | A-154 | D-166 | C-179 | S-187 | T-215 | V-228 |
| ADD25173 | E-105 | K-140 | V-143 | D-155 | C-168 | I-176 | V-204 | I-217 |
| ABF44291 | G-117 | E-151 | A-154 | D-166 | C-179 | I-187 | L-215 | A-227 |
| AEY93261 | E-114 | E-148 | A-151 | D-163 | C-176 | V-184 | V-212 | V-223 |
| 1NP2 | R-105 | F-139 | A-142 | D-154 | C-167 | T-175 | V-203 | V-214 |
| AEG34643 | R-105 | F-139 | A-142 | D-154 | C-167 | T-175 | V-203 | V-214 |
| AFR07907 | A-108 | L-142 | A-145 | D-157 | C-170 | N-178 | V-206 | V-218 |
| AEB43702 | D-122 | A-156 | A-159 | D-171 | C-184 | S-192 | V-220 | L-232 |
| BAL92882 | E-120 | E-154 | A-157 | D-169 | C-182 | S-190 | A-218 | I-229 |
| CCA59876 | E-116 | E-150 | A-153 | D-165 | C-178 | S-186 | V-214 | L-227 |
| ACU74192 | E-107 | Y-141 | A-144 | D-156 | C-169 | S-177 | L-205 | V-218 |
| ABV96319 | A-107 | E-141 | A-144 | D-156 | C-169 | N-177 | A-205 | V-216 |
| AF053528 | E-118 | E-152 | A-155 | D-167 | C-180 | S-188 | V-216 | C-229 |
| AFH40090 | R-105 | F-139 | A-142 | D-154 | C-167 | T-175 | V-203 | V-214 |
| ADL49193 | A-107 | E-141 | A-144 | D-156 | C-169 | S-177 | A-205 | L-216 |
| AFE08200 | S-105 | D-139 | V-142 | D-154 | C-167 | N-175 | V-203 | V-215 |
| AAN05441 | R-105 | F-139 | A-142 | D-154 | C-167 | T-175 | V-203 | V-214 |
| CAJ90043 | E-108 | E-142 | A-145 | D-157 | C-170 | I-178 | V-206 | V-217 |
| AEY89575 | E-110 | F-144 | A-147 | D-159 | C-172 | S-180 | V-208 | I-221 |
| AAF37730 | C-123 | K-157 | A-160 | D-172 | C-185 | S-193 | A-221 | I-237 |
| BAL98072 | A-107 | E-141 | V-144 | D-156 | C-169 | V-177 | V-205 | V-217 |
| AAN05440 | R-105 | F-139 | A-142 | D-154 | C-167 | T-175 | V-203 | V-214 |
| AEM77895 | E-104 | K-139 | V-142 | D-154 | C-167 | I-175 | V-203 | I-216 |
| ADB34272 | E-116 | E-150 | A-153 | D-165 | C-178 | S-186 | A-214 | V-224 |
| ABW87307 | R-105 | F-139 | A-142 | D-154 | C-167 | T-175 | V-203 | V-214 |
| AF322365 1 | R-105 | F-139 | A-142 | D-154 | C-167 | T-175 | V-203 | V-214 |
| CAC10107 | E-114 | Y-148 | A-151 | D-163 | C-176 | S-184 | T-212 | V-225 |
| AEB47478 | A-107 | E-141 | A-144 | D-156 | C-169 | N-177 | T-205 | V-216 |
| ADL45220 | E-122 | A-156 | A-159 | D-171 | C-184 | S-192 | V-220 | L-232 |
| ABK51908 | E-119 | Q-153 | A-156 | D-168 | C-181 | A-189 | V-217 | M-230 |
| CAN94460 | G-107 | D-142 | V-145 | D-157 | C-170 | K-178 | V-206 | V-218 |
| AEF18219 | E-104 | Y-138 | S-141 | D-153 | C-166 | I-174 | V-202 | I-215 |
| ADD39191 | E-128 | E-162 | A-165 | D-177 | C-190 | S-198 | T-226 | V-239 |
| ABP52811 | A-107 | E-141 | A-144 | D-156 | C-169 | N-177 | A-205 | V-216 |
| AEV88819 | E-135 | E-169 | G-172 | D-184 | C-197 | S-205 | A-233 | V-244 |
| AEY89570 | E-119 | H-153 | A-156 | D-168 | C-181 | S-189 | V-217 | T-230 |
| ADD01635 | E-104 | K-139 | V-142 | D-154 | C-167 | I-175 | V-203 | I-216 |
| CAB95278 | E-108 | E-142 | A-145 | D-157 | C-170 | I-178 | V-206 | V-217 |
| AEN13042 | S-108 | E-142 | G-145 | D-157 | C-170 | V-178 | M-206 | A-217 |
| ACY97750 | A-109 | F-143 | A-146 | D-158 | C-171 | S-179 | V-207 | C-219 |
| ADI10010 | E-106 | E-140 | A-143 | D-155 | C-168 | S-176 | A-204 | I-217 |
| CCB72805 | D-114 | Y-148 | A-151 | D-163 | C-176 | S-184 | V-212 | V-225 |
| ACY14034 | E-119 | Y-153 | A-156 | D-168 | C-181 | T-189 | T-217 | L-230 |
| AAZ55664 | A-110 | E-144 | A-147 | D-159 | C-172 | V-180 | V-208 | V-219 |
| ADW02698 | S-108 | E-142 | G-145 | D-157 | C-170 | V-178 | M-206 | A-217 |
| ABC33525 | A-106 | H-140 | A-143 | D-155 | C-168 | T-176 | L-204 | V-216 |
| ADB34282 | D-108 | Y-142 | A-145 | D-157 | C-170 | E-178 | V-206 | V-219 |
| AAM23648 | E-107 | K-142 | V-145 | D-157 | C-170 | I-178 | V-206 | I-219 |
| CCH86028 | E-117 | L-151 | A-154 | D-166 | C-179 | A-187 | T-215 | L-226 |
| ADV67544 | G-114 | L-148 | A-151 | G-163 | C-176 | I-184 | M-212 | L-224 |
| ACZ89862 | E-120 | E-154 | A-157 | D-169 | C-182 | S-190 | V-218 | I-229 |
| ADH66252 | T-108 | Y-142 | R-145 | D-157 | C-170 | N-178 | A-206 | V-218 |
| ADV80605 | E-104 | K-139 | V-142 | D-154 | C-167 | I-175 | V-203 | I-216 |
| ADW05507 | E-119 | D-153 | A-156 | D-168 | C-181 | S-189 | I-217 | T-230 |
| BAA86923 | R-105 | F-139 | A-142 | D-154 | C-167 | T-175 | V-203 | V-214 |
| AAN05438 | R-105 | F-139 | A-142 | D-154 | C-167 | T-175 | V-203 | V-214 |
| CAC16438 | E-119 | E-153 | A-156 | D-168 | C-181 | S-189 | V-217 | V-230 |
| CAJ89567 | D-119 | E-153 | A-156 | D-168 | C-181 | S-189 | V-217 | C-230 |
| AFD27167 | G-125 | H-159 | A-162 | D-174 | C-187 | I-195 | V-223 | V-235 |
| BAG20044 | E-126 | H-160 | A-163 | D-175 | C-188 | N-196 | A-224 | I-237 |
| ACU71435 | E-106 | Y-140 | A-143 | D-155 | C-168 | E-176 | T-204 | L-220 |
| ADG87563 | E-142 | Y-176 | A-179 | D-191 | V-204 | T-212 | A-240 | L-251 |
| ABI35984 | R-105 | F-139 | A-142 | D-154 | C-167 | T-175 | V-203 | V-214 |
| CAB42553 | R-105 | F-139 | A-142 | D-154 | C-167 | T-175 | V-203 | V-214 |
| 1UG6 | R-105 | F-139 | A-142 | D-154 | C-167 | T-175 | V-203 | V-214 |
| CAA91220 | E-107 | K-142 | V-145 | D-157 | C-170 | I-178 | V-206 | I-219 |
| AFK85369 | E-104 | Y-138 | S-141 | N-153 | C-166 | I-174 | V-202 | I-215 |
| AEN10177 | E-120 | D-154 | A-157 | D-169 | C-182 | S-190 | I-218 | T-231 |
| AEM87460 | E-106 | E-140 | A-143 | D-155 | C-168 | S-176 | A-204 | I-217 |
| AAZ81839 | A-107 | S-141 | L-144 | D-156 | C-169 | I-177 | V-205 | I-217 |
| ADU50085 | A-129 | D-163 | V-166 | D-178 | C-191 | S-199 | V-227 | L-239 |
| AEK47062 | E-119 | Y-153 | A-156 | D-168 | C-181 | H-189 | V-217 | F-230 |
| ABD68852 | G-119 | D-153 | V-156 | D-168 | C-181 | E-189 | I-217 | V-229 |
| ACL38401 | E-108 | Y-142 | A-145 | D-157 | C-170 | A-178 | A-206 | L-218 |
| ABS05424 | E-125 | E-159 | A-162 | D-174 | C-187 | S-195 | A-223 | V-235 |
| AEK43773 | C-102 | Y-136 | A-139 | D-151 | C-164 | G-172 | M-200 | A-212 |
| AEV87561 | E-96 | Y-130 | A-133 | D-145 | C-158 | H-166 | Y-194 | V-204 |
| ACZ86244 | E-116 | K-150 | A-153 | D-165 | C-178 | S-186 | T-214 | I-226 |
| AEB46173 | R-107 | A-141 | A-144 | D-156 | C-169 | S-177 | G-205 | L-217 |
| BAG21567 | E-121 | E-155 | A-158 | D-170 | C-183 | S-191 | T-219 | T-232 |
| ABX05041 | E-107 | E-141 | V-144 | D-156 | C-169 | I-177 | V-205 | V-217 |
| AFR09943 | A-124 | H-158 | A-161 | D-173 | C-186 | V-194 | A-222 | V-238 |
| ACZ89285 | A-115 | E-149 | A-152 | D-163 | C-176 | E-184 | A-212 | V-225 |
| ACV58907 | E-104 | S-138 | L-141 | D-153 | C-166 | I-174 | V-202 | I-214 |
| ADG89462 | A-99 | A-133 | A-136 | D-148 | V-161 | L-169 | V-197 | V-208 |
| ADU09756 | A-124 | E-158 | A-161 | D-173 | C-186 | E-194 | V-222 | V-235 |
| ACZ90607 | A-99 | Y-133 | A-136 | D-148 | V-161 | M-169 | A-197 | V-208 |
| ADG73989 | R-113 | E-147 | A-150 | D-162 | C-175 | S-183 | A-211 | V-224 |
| AEG45154 | E-138 | Y-172 | A-175 | D-187 | C-200 | S-208 | V-236 | L-248 |
| AEJ43907 | K-104 | S-138 | L-141 | D-153 | C-166 | I-174 | V-202 | I-214 |
| ACZ20790 | S-123 | Y-157 | A-160 | D-172 | C-185 | A-193 | T-221 | L-233 |
| CCB77455 | A-109 | H-148 | A-151 | D-163 | V-176 | L-184 | A-212 | V-222 |
| ADL46625 | E-121 | Y-155 | A-158 | D-170 | C-183 | Y-191 | T-219 | L-232 |
| ADU10772 | E-121 | Y-155 | A-158 | D-170 | C-183 | Y-191 | T-219 | L-232 |
| ADD45899 | R-109 | L-143 | A-146 | D-158 | C-171 | S-179 | T-207 | I-219 |
| CCA53920 | E-120 | H-154 | A-157 | D-169 | C-182 | E-190 | V-218 | L-231 |
| BAJ28512 | T-120 | H-154 | A-157 | D-169 | C-182 | S-190 | V-218 | V-231 |
| ACL70277 | N-107 | K-141 | A-144 | G-156 | V-169 | F-177 | V-205 | I-217 |
| CBG72797 | E-114 | H-148 | A-151 | D-163 | C-176 | S-184 | V-212 | I-225 |
| AEW05616 | E-107 | E-141 | N-144 | R-156 | C-169 | L-177 | V-205 | I-217 |
| AEB46623 | G-105 | H-139 | A-142 | D-154 | I-167 | T-175 | V-203 | V-214 |
| 1GNX | E-119 | E-153 | A-156 | D-168 | C-181 | S-189 | V-217 | C-230 |
| ACV76621 | G-109 | L-143 | A-146 | D-158 | C-171 | S-179 | G-207 | L-220 |
| ACZ31628 | E-139 | Y-173 | A-176 | D-188 | C-201 | G-209 | T-237 | V-249 |
| ADH67953 | T-107 | Y-141 | A-144 | D-156 | C-169 | V-177 | T-205 | V-216 |
| ADB34290 | E-120 | Y-154 | A-157 | D-169 | C-182 | Y-190 | T-218 | I-231 |
| ADU09106 | G-134 | A-168 | A-171 | D-183 | I-196 | M-204 | V-232 | V-243 |
| CAA82733 | E-119 | E-153 | A-156 | D-168 | C-181 | S-189 | V-217 | C-230 |
| CBG67455 | A-136 | E-170 | A-173 | D-185 | C-198 | S-206 | V-234 | V-247 |
| CAN00920 | G-126 | Y-160 | A-163 | D-175 | C-188 | A-196 | V-224 | F-237 |
| ADX71280 | E-108 | Y-142 | A-145 | D-157 | C-170 | A-178 | A-206 | L-218 |
| ACQ81085 | A-122 | Q-156 | V-159 | D-171 | C-184 | G-192 | I-220 | L-232 |
| 3AHX | E-107 | D-141 | V-144 | D-156 | V-169 | L-177 | V-205 | I-217 |
| ACU35632 | E-118 | H-152 | A-155 | D-167 | C-180 | V-188 | T-216 | F-229 |
| AEV86556 | A-108 | D-142 | A-145 | D-157 | C-170 | E-178 | V-206 | V-218 |
| AEG45006 | A-118 | L-152 | A-155 | D-167 | C-180 | S-188 | V-216 | T-228 |
| BAG18801 | E-120 | L-154 | A-157 | D-169 | C-182 | H-190 | A-218 | L-232 |
| ADI03707 | A-118 | E-152 | A-155 | D-167 | C-180 | E-188 | V-216 | V-227 |
| CAQ00266 | G-126 | Y-160 | A-163 | D-175 | C-188 | A-196 | V-224 | F-237 |
| ADD43929 | A-108 | A-142 | A-145 | D-157 | E-170 | L-178 | T-206 | V-217 |
| ADJ49823 | E-103 | S-137 | A-140 | D-152 | C-165 | S-173 | A-201 | F-214 |
| ABX05062 | G-109 | L-143 | A-146 | E-158 | V-171 | Q-179 | T-207 | I-219 |
| ADL48215 | G-110 | A-144 | A-147 | D-159 | I-172 | M-180 | V-208 | V-219 |
| ACU70272 | S-111 | H-145 | A-148 | D-160 | I-173 | F-181 | V-209 | V-220 |
| ADG88606 | E-116 | K-150 | A-153 | D-165 | C-178 | S-186 | V-214 | I-226 |
| AEW47954 | E-108 | G-142 | A-145 | D-157 | C-170 | S-178 | M-206 | A-219 |
| ACD20223 | R-132 | Y-166 | A-169 | G-181 | C-194 | N-202 | V-230 | V-242 |
| AFC28171 | Q-106 | A-140 | E-143 | D-155 | C-168 | M-176 | V-204 | I-216 |
| ADW03239 | E-119 | L-153 | A-156 | D-168 | C-181 | Y-189 | A-217 | L-231 |
| CBT74727 | E-111 | Y-145 | V-148 | D-160 | C-173 | A-181 | T-209 | I-220 |
| CAM04686 | E-109 | A-143 | A-146 | D-158 | C-171 | S-179 | M-207 | F-220 |
| ACZ20966 | G-112 | E-146 | A-149 | D-161 | C-174 | S-182 | A-210 | V-223 |
| AEV38153 | L-122 | D-157 | V-160 | A-172 | T-185 | L-193 | V-222 | I-234 |
| BAJ26623 | G-108 | H-142 | A-145 | D-157 | V-170 | L-178 | A-206 | V-216 |
| BAL91665 | E-96 | Y-130 | A-133 | D-145 | C-158 | H-166 | A-194 | V-204 |
| ACV09397 | G-110 | Y-144 | A-147 | T-159 | C-172 | S-180 | G-208 | V-221 |
| ADG20157 | G-107 | Y-142 | A-145 | D-157 | C-170 | T-178 | L-206 | L-218 |
| ADC61565 | A-106 | H-140 | A-143 | D-155 | V-168 | D-176 | V-204 | I-215 |
| ACM66669 | E-111 | Y-145 | L-148 | D-160 | C-173 | A-181 | T-209 | I-220 |
| ABD68843 | G-114 | Q-148 | V-151 | D-163 | V-176 | I-184 | L-212 | L-224 |
| ABS61373 | E-107 | M-141 | R-144 | D-156 | C-169 | T-177 | V-205 | I-217 |
| CBA30283 | G-121 | Y-156 | A-159 | D-171 | C-184 | Q-192 | L-220 | L-232 |
| AAA22266 | E-107 | D-141 | A-144 | G-156 | C-169 | L-177 | V-205 | I-217 |
| CAA42814 | L-107 | D-141 | T-144 | D-156 | V-169 | L-177 | V-205 | I-217 |
| ABN51453 | L-130 | D-164 | T-167 | D-179 | V-192 | L-200 | V-228 | I-240 |
| ACZ00292 | G-104 | H-138 | A-141 | D-153 | V-166 | F-174 | V-202 | I-214 |
| AEI12946 | R-113 | E-147 | A-150 | D-162 | C-175 | S-183 | A-211 | I-224 |
| ABS15474 | D-122 | D-156 | A-159 | D-171 | T-184 | S-192 | V-221 | V-233 |
| ADL51094 | E-107 | D-141 | V-144 | D-156 | V-169 | L-177 | V-205 | I-217 |
| AFG34202 | E-107 | L-141 | R-144 | D-156 | C-169 | T-177 | V-205 | V-217 |
| AAN60220 | E-107 | L-141 | R-144 | D-156 | C-169 | T-177 | V-205 | V-217 |

TABLE 9B

| Corresp. | Sequence Name | | | | | | | | |
|---|---|---|---|---|---|---|---|---|---|
| Sequence Pos. | T219 | K231 | D243 | M246 | F292 | S296 | T297 | D302 | H315 |
| SEQ ID NO. | T-219 | K231 | D-243 | M-246 | F-292 | S-296 | T-297 | D-302 | H-315 |
| 379/380 | | | | | | | | | |
| SEQ ID NO. 378 | T-222 | — | D-245 | F-248 | I-293 | S-297 | R-298 | H-303 | H-313 |
| US8101393-0094 | T-219 | K-231 | D-243 | M-246 | F-292 | S-296 | T-297 | D-302 | H-315 |
| US8101393-0388 | T-219 | K-231 | D-243 | M-246 | F-292 | S-296 | T-297 | D-302 | H-315 |
| US8101393-0172 | T-222 | — | D-245 | F-248 | I-292 | S-296 | R-297 | H-302 | R-314 |
| JP2011205992- | V-221 | — | D-244 | V-247 | V-292 | E-296 | R-297 | D-302 | D-316 |
| 0018 | | | | | | | | | |
| JP2011205992- | T-220 | — | D-243 | Q-246 | I-290 | S-294 | R-295 | N-300 | R-312 |
| 0023 | | | | | | | | | |
| JP2011205992- | T-222 | K-234 | D-245 | F-248 | V-293 | S-297 | R-298 | A-303 | Q-317 |
| 0022 | | | | | | | | | |
| US20110214199- | V-222 | R-234 | D-245 | V-248 | L-292 | F-296 | P-297 | D-302 | R-316 |
| 58656 | | | | | | | | | |
| US20110214199- | V-238 | R-250 | D-261 | T-264 | L-308 | F-312 | P-313 | A-318 | R-332 |
| 62406 | | | | | | | | | |
| US20110214199- | T-218 | — | D-241 | F-244 | I-296 | S-300 | R-301 | S-306 | H-320 |
| 47919 | | | | | | | | | |
| US20110214199- | T-217 | — | D-240 | A-243 | V-290 | M-294 | R-295 | A-300 | G-319 |
| 60662 | | | | | | | | | |
| US20110214199- | T-224 | — | D-247 | S-250 | V-297 | F-301 | R-302 | F-307 | D-326 |
| 36660 | | | | | | | | | |
| US20110214199- | S-231 | — | D-254 | A-257 | L-304 | T-308 | P-309 | A-314 | D-340 |
| 17908 | | | | | | | | | |
| US20110214199- | V-217 | — | D-235 | H-238 | V-281 | A-285 | P-286 | P-291 | P-302 |
| 25308 | | | | | | | | | |
| US20110214199- | V-217 | — | D-235 | H-238 | V-281 | A-285 | P-286 | P-291 | P-302 |
| 64004 | | | | | | | | | |
| US20110214199- | A-240 | — | D-263 | R-266 | V-313 | N-317 | P-318 | G-323 | H-346 |
| 25023 | | | | | | | | | |
| US20110214199- | V-217 | — | D-235 | H-238 | V-281 | A-285 | P-286 | P-291 | P-302 |
| 52644 | | | | | | | | | |
| US20110214199- | S-228 | — | D-251 | S-254 | L-301 | T-305 | P-306 | A-311 | D-337 |
| 13862 | | | | | | | | | |
| US20110214199- | T-222 | K-234 | D-245 | A-248 | V-296 | T-300 | R-301 | Y-306 | N-316 |
| 29446 | | | | | | | | | |
| US20110214199- | V-217 | — | D-235 | H-238 | V-281 | A-285 | P-286 | P-291 | P-302 |
| 5988 | | | | | | | | | |
| US20110214199- | V-217 | — | D-235 | H-238 | V-281 | A-285 | P-286 | P-291 | P-302 |
| 23246 | | | | | | | | | |
| US20110214199- | T-220 | D-232 | D-243 | N-246 | I-293 | T-297 | R-298 | N-303 | S-313 |
| 12000 | | | | | | | | | |
| US20110214199- | T-220 | K-232 | D-243 | V-246 | V-291 | T-295 | T-297 | D-302 | Y-311 |
| 56949 | | | | | | | | | |
| US8101393-0272 | T-231 | — | D-254 | G-257 | I-304 | S-308 | P-309 | A-314 | R-328 |
| US8101393-0014 | T-224 | S-236 | D-247 | Y-250 | V-301 | T-305 | R-306 | A-311 | F-323 |
| US8101393-0398 | T-235 | — | D-258 | A-261 | V-308 | S-312 | R-313 | A-318 | E-340 |
| US8101393-0492 | T-235 | — | D-258 | A-261 | V-308 | S-312 | R-313 | A-318 | E-340 |
| US8101393-0400 | T-235 | — | D-258 | A-261 | I-308 | S-312 | R-313 | A-318 | D-340 |
| US8101393-0266 | S-230 | — | D-253 | G-256 | I-303 | T-307 | P-308 | H-313 | M-339 |
| US8101393-0366 | V-215 | — | D-233 | H-236 | V-279 | T-283 | R-284 | R-289 | P-300 |
| US8101393-0342 | T-220 | S-232 | D-243 | F-246 | V-298 | T-302 | R-303 | S-308 | L-322 |
| US8101393-0356 | V-220 | S-232 | H-243 | F-246 | V-291 | S-295 | R-296 | D-301 | N-312 |
| US8101393-0320 | A-220 | K-232 | F-243 | A-246 | F-293 | S-297 | S-298 | Y-303 | S-316 |
| US7314974-19451 | T-216 | S-228 | D-239 | F-242 | I-294 | S-298 | R-299 | S-304 | H-318 |
| US7314974-8251 | A-215 | — | D-238 | R-241 | V-288 | N-292 | P-293 | G-298 | H-321 |
| US7630836-9340 | V-222 | R-234 | D-245 | V-248 | L-292 | F-296 | P-297 | D-302 | R-316 |
| US7630836-12787 | S-231 | — | D-254 | A-257 | L-304 | T-308 | P-309 | A-314 | D-340 |
| US20090220480- | V-218 | S-230 | D-241 | F-244 | I-296 | S-300 | R-301 | S-306 | M-320 |
| 0018 | | | | | | | | | |
| US20120034253- | V-217 | — | D-235 | H-238 | V-281 | A-285 | P-286 | P-291 | P-302 |
| 0080 | | | | | | | | | |
| US20120034253- | V-217 | — | D-235 | H-238 | V-281 | A-285 | P-286 | P-291 | P-302 |
| 0077 | | | | | | | | | |
| US20120034253- | V-217 | — | D-235 | H-238 | V-281 | A-285 | P-286 | P-291 | P-302 |
| 0078 | | | | | | | | | |
| WO2012016960- | T-247 | — | D-271 | A-274 | I-321 | N-325 | P-326 | A-331 | G-346 |
| 11369 | | | | | | | | | |
| US20120015408- | T-222 | K-234 | D-245 | A-248 | V-296 | T-300 | R-301 | Y-306 | N-316 |
| 0002 | | | | | | | | | |
| US20120015408- | T-222 | K-234 | D-245 | A-248 | V-296 | T-300 | R-301 | Y-306 | N-316 |
| 0004 | | | | | | | | | |
| US20120015408- | T-222 | K-234 | D-245 | A-248 | V-296 | T-300 | R-301 | Y-306 | N-316 |
| 0006 | | | | | | | | | |
| US20120015408- | T-222 | K-234 | D-245 | A-248 | V-296 | T-300 | R-301 | Y-306 | N-316 |
| 0008 | | | | | | | | | |
| US20120015408- | T-222 | K-234 | D-245 | A-248 | V-296 | T-300 | R-301 | Y-306 | N-316 |
| 0010 | | | | | | | | | |
| US20120015408- | T-222 | K-234 | D-245 | A-248 | V-296 | T-300 | R-301 | Y-306 | N-316 |
| 0012 | | | | | | | | | |
| US20120015408- | T-222 | K-234 | D-245 | A-248 | V-296 | T-300 | R-301 | Y-306 | N-316 |
| 0014 | | | | | | | | | |
| US20120015408- | T-222 | K-234 | D-245 | A-248 | V-296 | T-300 | R-301 | Y-306 | N-316 |
| 0016 | | | | | | | | | |
| US20120015408- | T-222 | K-234 | D-245 | A-248 | V-296 | T-300 | R-301 | Y-306 | N-316 |
| 0018 | | | | | | | | | |
| US20120015408- | T-222 | K-234 | D-245 | A-248 | V-296 | T-300 | R-301 | Y-306 | N-316 |
| 0020 | | | | | | | | | |
| US20120015408- | T-222 | K-234 | D-245 | A-248 | V-296 | T-300 | R-301 | Y-306 | N-316 |
| 0022 | | | | | | | | | |
| US20120015408- | T-222 | K-234 | D-245 | A-248 | V-296 | T-300 | R-301 | Y-306 | N-316 |
| 0026 | | | | | | | | | |
| US20120015408- | T-222 | K-234 | D-245 | A-248 | V-296 | T-300 | R-301 | Y-306 | N-316 |
| 0028 | | | | | | | | | |
| US20120015408- | T-222 | K-234 | D-245 | A-248 | V-296 | T-300 | R-301 | Y-306 | N-316 |
| 0030 | | | | | | | | | |
| US20120015408- | T-222 | K-234 | D-245 | A-248 | V-296 | T-300 | R-301 | Y-306 | N-316 |
| 0032 | | | | | | | | | |
| US20120015408- | T-222 | K-234 | D-245 | A-248 | V-296 | T-300 | R-301 | Y-306 | N-316 |
| 0034 | | | | | | | | | |
| US20120015408- | T-222 | K-234 | D-245 | A-248 | V-296 | T-300 | R-301 | Y-306 | N-316 |
| 0036 | | | | | | | | | |
| US20120015408- | T-222 | K-234 | D-245 | A-248 | V-296 | T-300 | R-301 | Y-306 | N-316 |
| 0038 | | | | | | | | | |
| US20120015408- | T-222 | K-234 | D-245 | A-248 | V-296 | T-300 | R-301 | Y-306 | N-316 |
| 0040 | | | | | | | | | |
| US20120015408- | T-222 | K-234 | D-245 | A-248 | V-296 | T-300 | R-301 | Y-306 | N-316 |
| 0042 | | | | | | | | | |
| US20120015408- | T-222 | K-234 | D-245 | A-248 | V-296 | T-300 | R-301 | Y-306 | N-316 |
| 0044 | | | | | | | | | |
| US20120015408- | T-222 | K-234 | D-245 | A-248 | V-296 | T-300 | R-301 | Y-306 | N-316 |
| 0046 | | | | | | | | | |
| US20120015408- | T-222 | K-234 | D-245 | A-248 | V-296 | T-300 | R-301 | Y-306 | N-316 |
| 0048 | | | | | | | | | |
| US20120015408- | T-222 | K-234 | D-245 | A-248 | V-296 | T-300 | R-301 | Y-306 | N-316 |
| 0050 | | | | | | | | | |
| US20120015408- | T-222 | K-234 | D-245 | A-248 | V-296 | T-300 | R-301 | Y-306 | N-316 |
| 0052 | | | | | | | | | |
| US20120015408- | T-222 | K-234 | D-245 | A-248 | V-296 | T-300 | R-301 | Y-306 | N-316 |
| 0054 | | | | | | | | | |
| US20120015408- | T-222 | K-234 | D-245 | A-248 | V-296 | T-300 | R-301 | Y-306 | N-316 |
| 0056 | | | | | | | | | |
| US20120015408- | T-222 | K-234 | D-245 | A-248 | V-296 | T-300 | R-301 | Y-306 | N-316 |
| 0058 | | | | | | | | | |
| US20120015408- | T-222 | K-234 | D-245 | A-248 | V-296 | T-300 | R-301 | Y-306 | N-316 |
| 0060 | | | | | | | | | |
| US20120015408- | T-222 | K-234 | D-245 | A-248 | V-296 | T-300 | R-301 | Y-306 | N-316 |
| 0062 | | | | | | | | | |
| US20120015408- | T-222 | K-234 | D-245 | A-248 | V-296 | T-300 | R-301 | Y-306 | N-316 |
| 0064 | | | | | | | | | |
| US20120015408- | T-222 | K-234 | D-245 | A-248 | V-296 | T-300 | R-301 | Y-306 | N-316 |
| 0066 | | | | | | | | | |
| US20120015408- | T-222 | K-234 | D-245 | A-248 | V-296 | T-300 | R-301 | Y-306 | N-316 |
| 0068 | | | | | | | | | |
| US20120015408- | T-222 | K-234 | D-245 | A-248 | V-296 | T-300 | R-301 | Y-306 | N-316 |
| 0070 | | | | | | | | | |
| US20120015408- | T-222 | K-234 | D-245 | A-248 | V-296 | T-300 | R-301 | Y-306 | N-316 |
| 0072 | | | | | | | | | |
| US20120015408- | T-222 | K-234 | D-245 | A-248 | V-296 | T-300 | R-301 | Y-306 | N-316 |
| 0074 | | | | | | | | | |
| US20120015408- | T-222 | K-234 | D-245 | A-248 | V-296 | T-300 | R-301 | Y-306 | N-316 |
| 0076 | | | | | | | | | |
| US20120015408- | T-222 | K-234 | D-245 | A-248 | V-296 | T-300 | R-301 | Y-306 | N-316 |
| 0078 | | | | | | | | | |
| US20120015408- | T-222 | K-234 | D-245 | A-248 | V-296 | T-300 | R-301 | Y-306 | N-316 |
| 0080 | | | | | | | | | |
| US20120015408- | T-222 | K-234 | D-245 | A-248 | V-296 | T-300 | R-301 | Y-306 | N-316 |
| 0082 | | | | | | | | | |
| US20120015408- | A-222 | K-234 | D-245 | A-248 | V-296 | T-300 | R-301 | Y-306 | N-316 |
| 0086 | | | | | | | | | |
| US20120015408- | T-222 | K-234 | D-245 | A-248 | V-296 | T-300 | R-301 | Y-306 | N-316 |
| 0084 | | | | | | | | | |
| US20120015408- | T-222 | K-234 | D-245 | A-248 | V-296 | T-300 | R-301 | Y-306 | N-316 |
| 0088 | | | | | | | | | |
| US20120015408- | T-222 | K-234 | D-245 | A-248 | V-296 | T-300 | R-301 | Y-306 | N-316 |
| 0090 | | | | | | | | | |
| US20120015408- | T-222 | K-234 | D-245 | A-248 | V-296 | T-300 | R-301 | Y-306 | N-316 |
| 0092 | | | | | | | | | |
| US20120015408- | T-222 | K-234 | D-245 | A-248 | V-296 | T-300 | R-301 | Y-306 | N-316 |
| 0094 | | | | | | | | | |
| US20120015408- | T-222 | K-234 | D-245 | A-248 | V-296 | T-300 | R-301 | Y-306 | N-316 |
| 0096 | | | | | | | | | |
| US20120015408- | T-222 | K-234 | D-245 | A-248 | V-296 | T-300 | R-301 | Y-306 | N-316 |
| 0098 | | | | | | | | | |
| US20120015408- | T-222 | K-234 | D-245 | A-248 | V-296 | T-300 | R-301 | Y-306 | N-316 |
| 0100 | | | | | | | | | |
| US20120015408- | T-222 | K-234 | D-245 | A-248 | V-296 | T-300 | R-301 | Y-306 | N-316 |
| 0102 | | | | | | | | | |
| US20120015408- | T-222 | K-234 | D-245 | A-248 | V-296 | T-300 | R-301 | Y-306 | N-316 |
| 0104 | | | | | | | | | |
| US20120015408- | T-222 | K-234 | D-245 | A-248 | V-296 | T-300 | R-301 | Y-306 | N-316 |
| 0106 | | | | | | | | | |
| US20120015408- | T-222 | K-234 | D-245 | A-248 | V-296 | T-300 | R-301 | Y-306 | N-316 |
| 0108 | | | | | | | | | |
| US20120015408- | T-222 | K-234 | D-245 | A-248 | V-296 | T-300 | R-301 | Y-306 | N-316 |
| 0110 | | | | | | | | | |
| US20120015408- | T-222 | K-234 | D-245 | A-248 | V-296 | T-300 | R-301 | Y-306 | N-316 |
| 0112 | | | | | | | | | |
| US20120015408- | T-222 | K-234 | D-245 | A-248 | V-296 | T-300 | R-301 | Y-306 | N-316 |
| 0114 | | | | | | | | | |
| US20120015408- | T-222 | K-234 | D-245 | A-248 | V-296 | T-300 | R-301 | Y-306 | N-316 |
| 0116 | | | | | | | | | |
| US20120015408- | T-222 | K-234 | D-245 | A-248 | V-296 | T-300 | R-301 | Y-306 | N-316 |
| 0118 | | | | | | | | | |
| US20120015408- | T-222 | K-234 | D-245 | A-248 | V-296 | T-300 | R-301 | Y-306 | N-316 |
| 0120 | | | | | | | | | |
| US20120015408- | T-222 | K-234 | D-245 | A-248 | V-296 | T-300 | R-301 | Y-306 | N-316 |
| 0122 | | | | | | | | | |
| US20120015408- | T-222 | K-234 | D-245 | A-248 | V-296 | T-300 | R-301 | Y-306 | N-316 |
| 0124 | | | | | | | | | |
| US8202716-0464 | T-220 | N-232 | D-243 | Q-246 | V-294 | T-298 | R-299 | D-304 | H-315 |
| US20110262988- | A-220 | K-232 | F-243 | A-246 | F-293 | S-297 | S-298 | Y-303 | S-316 |
| 0002 | | | | | | | | | |
| US20110262988- | A-243 | K-255 | F-266 | A-269 | F-316 | S-320 | S-321 | Y-326 | S-339 |
| 0003 | | | | | | | | | |
| US20110151538- | A-272 | K-284 | F-295 | A-298 | F-345 | S-349 | S-350 | Y-355 | S-368 |
| 0130 | | | | | | | | | |
| US20100003234- | T-221 | E-232 | D-244 | M-247 | I-294 | T-298 | R-299 | A-304 | P-315 |
| 0048 | | | | | | | | | |
| US6184018-0012 | A-220 | K-232 | F-243 | A-246 | F-293 | S-297 | S-298 | Y-303 | S-316 |
| US6377893-0063 | A-215 | K-227 | F-238 | A-241 | F-288 | S-292 | S-293 | Y-298 | S-311 |
| ACJ34717 | T-230 | K-242 | D-254 | M-257 | F-303 | S-307 | T-308 | D-313 | H-326 |
| ADD27066 | T-220 | — | D-243 | Q-246 | I-290 | S-294 | R-295 | N-300 | R-312 |
| ACZ42845 | T-227 | S-239 | D-250 | M-253 | V-298 | S-302 | P-303 | H-308 | R-321 |
| ABQ91969 | T-219 | S-231 | D-242 | F-245 | V-292 | S-296 | R-297 | D-302 | R-315 |
| ABU56651 | T-219 | S-231 | D-242 | F-245 | V-292 | S-296 | R-297 | D-302 | R-315 |
| AEY92801 | V-224 | R-236 | D-247 | V-250 | L-294 | F-298 | P-299 | D-304 | R-318 |
| CAD55382 | V-238 | R-250 | D-261 | T-264 | L-308 | F-312 | P-313 | A-318 | R-332 |
| AD073143 | T-220 | S-232 | D-243 | F-246 | I-298 | N-302 | R-303 | S-308 | F-322 |
| CCA60311 | T-231 | — | D-254 | G-257 | I-304 | S-308 | P-309 | A-314 | R-328 |
| ADI15206 | V-231 | A-243 | D-254 | F-257 | V-302 | S-306 | R-307 | H-312 | P-323 |
| ADI12494 | V-224 | R-236 | D-247 | T-250 | L-294 | F-298 | P-299 | D-304 | R-318 |
| ACU35736 | T-219 | — | D-243 | Q-246 | V-293 | T-297 | E-298 | S-303 | H-327 |
| BAC69512 | V-222 | R-234 | D-245 | V-248 | L-292 | F-296 | P-297 | D-302 | R-316 |
| AEN08263 | V-224 | R-236 | D-247 | T-250 | L-294 | F-298 | P-299 | D-304 | V-318 |
| CCA53915 | V-248 | R-260 | D-271 | T-274 | L-318 | F-322 | P-323 | D-328 | L-342 |
| CAJ88063 | V-224 | R-236 | D-247 | T-250 | L-294 | F-298 | P-299 | D-304 | L-318 |
| ABF87202 | T-218 | S-230 | D-241 | F-244 | I-296 | S-300 | R-301 | S-306 | H-320 |
| BAJ30040 | V-221 | S-233 | D-244 | I-247 | L-291 | F-295 | R-296 | D-301 | G-315 |
| ACO44852 | V-222 | S-234 | D-245 | A-248 | V-293 | T-297 | R-298 | A-303 | P-309 |
| AFO59750 | T-223 | — | D-246 | M-249 | V-296 | S-300 | R-301 | A-306 | D-321 |
| BAJ31549 | T-246 | — | D-269 | A-272 | I-319 | T-323 | P-324 | A-329 | H-354 |
| ACY97307 | A-236 | — | D-259 | Q-262 | I-309 | T-313 | R-314 | G-319 | H-343 |
| ACM06095 | T-220 | Q-232 | D-243 | L-246 | I-290 | A-294 | P-295 | A-300 | E-314 |
| AEI64652 | T-218 | S-230 | D-241 | F-244 | I-296 | S-300 | R-301 | S-306 | H-320 |
| BAG17581 | V-238 | S-250 | D-261 | I-264 | L-308 | F-312 | R-313 | A-318 | V-332 |
| AEM83530 | V-224 | R-236 | D-247 | T-250 | L-294 | F-298 | P-299 | D-304 | R-318 |
| ADG89307 | T-227 | A-239 | D-250 | V-253 | V-300 | Y-304 | R-305 | A-310 | V-332 |
| ABG04991 | T-217 | — | D-240 | A-243 | V-290 | M-294 | R-295 | A-300 | G-319 |
| ABP54026 | T-236 | — | D-259 | A-262 | I-309 | S-313 | R-314 | A-319 | D-341 |
| ABV97405 | T-236 | — | D-259 | A-262 | I-309 | S-313 | R-314 | A-319 | H-341 |
| ADH60167 | T-219 | K-231 | D-242 | S-245 | V-293 | T-297 | R-298 | Y-303 | N-313 |
| ADV80493 | T-219 | K-231 | D-242 | A-245 | V-293 | T-297 | R-298 | Y-303 | N-313 |
| AEM77729 | T-219 | K-231 | D-242 | S-245 | V-293 | T-297 | R-298 | Y-303 | N-313 |
| ABK71329 | T-224 | — | D-247 | S-250 | V-297 | F-301 | R-302 | F-307 | D-326 |
| ACZ89864 | V-231 | V-243 | D-262 | L-265 | I-312 | N-316 | P-317 | S-322 | D-337 |
| BAC72965 | S-231 | — | D-254 | A-257 | L-304 | T-308 | P-309 | A-314 | D-340 |
| ADD25173 | T-220 | K-232 | D-243 | A-246 | V-294 | T-298 | R-299 | Y-304 | N-314 |
| ABF44291 | V-230 | R-242 | D-253 | F-256 | V-301 | S-305 | R-306 | A-311 | P-317 |
| AEY93261 | T-226 | S-238 | D-249 | H-252 | I-300 | R-304 | P-305 | D-310 | G-332 |
| 1NP2 | V-217 | — | D-235 | H-238 | V-281 | A-285 | P-286 | P-291 | P-302 |
| AEG34643 | V-217 | — | D-235 | H-238 | V-281 | A-285 | P-286 | P-291 | P-302 |
| AFR07907 | A-221 | — | D-244 | R-247 | V-294 | T-298 | P-299 | G-304 | E-327 |
| AEB43702 | T-235 | — | D-258 | A-261 | V-308 | S-312 | R-313 | A-318 | D-340 |
| BAL92882 | T-232 | — | D-256 | A-259 | I-306 | T-310 | P-311 | A-316 | G-331 |
| CCA59876 | S-230 | — | D-253 | G-256 | I-303 | T-307 | P-308 | H-313 | M-339 |
| ACU74192 | T-221 | — | D-244 | Q-247 | I-294 | N-298 | P-299 | A-304 | G-332 |
| ABV96319 | T-219 | — | D-243 | Q-246 | V-293 | T-297 | P-298 | G-303 | G-319 |
| AF053528 | T-232 | — | D-255 | A-258 | V-305 | T-309 | P-310 | E-315 | Q-340 |
| AFH40090 | V-217 | — | D-235 | H-238 | V-281 | A-285 | P-286 | P-291 | P-302 |
| ADL49193 | T-219 | — | D-243 | H-246 | L-293 | A-297 | P-298 | G-303 | G-318 |
| AFE08200 | T-218 | S-230 | D-241 | F-244 | V-296 | S-300 | R-301 | S-306 | F-320 |
| AAN05441 | V-217 | — | D-235 | H-238 | V-281 | A-285 | P-286 | P-291 | P-302 |
| CAJ90043 | T-220 | S-232 | D-243 | H-246 | V-294 | R-298 | P-299 | G-304 | R-326 |
| AEY89575 | S-224 | — | D-247 | A-250 | L-297 | T-301 | P-302 | A-307 | D-333 |
| AAF37730 | A-240 | — | D-263 | R-266 | V-313 | N-317 | P-318 | G-323 | H-346 |
| BAL98072 | T-220 | T-232 | D-243 | F-246 | V-298 | S-302 | R-303 | A-308 | P-320 |
| AAN05440 | V-217 | — | D-235 | H-238 | V-281 | A-285 | P-286 | P-291 | P-302 |
| AEM77895 | T-219 | K-231 | D-242 | S-245 | V-293 | T-297 | R-298 | Y-303 | N-313 |
| ADB34272 | T-227 | — | D-250 | Q-253 | V-300 | S-304 | R-305 | G-310 | H-335 |
| ABW87307 | V-217 | — | D-235 | H-238 | V-281 | A-285 | P-286 | P-291 | P-302 |
| AF322365 1 | V-217 | — | D-235 | H-238 | V-281 | A-285 | P-286 | P-291 | P-302 |
| CAC10107 | S-228 | — | D-251 | S-254 | L-301 | T-305 | P-306 | A-311 | D-337 |
| AEB47478 | T-219 | — | D-243 | H-246 | I-293 | A-297 | P-298 | G-303 | A-319 |
| ADL45220 | T-235 | — | D-258 | A-261 | V-308 | S-312 | R-313 | A-318 | D-340 |
| ABK51908 | T-233 | — | D-256 | Q-259 | V-306 | N-310 | P-311 | H-316 | D-341 |
| CAN94460 | T-221 | S-233 | D-244 | F-247 | L-299 | S-303 | R-304 | S-309 | H-323 |
| AEF18219 | T-218 | K-230 | D-241 | S-244 | I-292 | S-296 | R-297 | Y-302 | A-312 |
| ADD39191 | G-242 | — | D-265 | A-268 | L-315 | T-319 | P-320 | A-325 | H-351 |
| ABP52811 | T-219 | — | D-243 | H-246 | V-293 | A-297 | P-298 | G-303 | G-319 |
| AEV88819 | T-247 | — | D-271 | A-274 | I-321 | N-325 | P-326 | A-331 | G-346 |
| AEY89570 | T-233 | — | D-256 | G-259 | V-307 | S-311 | P-312 | L-317 | H-343 |
| ADD01635 | T-219 | K-231 | D-242 | S-245 | V-293 | T-297 | R-298 | Y-303 | N-313 |
| CAB95278 | T-220 | S-232 | D-243 | H-246 | V-294 | R-298 | P-299 | D-304 | R-326 |
| AEN13042 | T-220 | S-232 | D-243 | H-246 | I-294 | R-298 | P-299 | A-304 | S-326 |
| ACY97750 | T-222 | — | D-243 | A-246 | V-293 | A-297 | P-298 | R-303 | E-329 |
| ADI10010 | S-220 | — | D-243 | A-246 | I-293 | T-297 | P-298 | A-303 | R-329 |
| CCB72805 | C-228 | — | D-251 | G-254 | V-301 | T-305 | P-306 | A-311 | R-337 |
| ACY14034 | T-233 | — | D-256 | G-259 | I-306 | F-310 | G-311 | G-316 | R-333 |
| AAZ55664 | T-222 | S-234 | E-245 | H-248 | I-295 | R-299 | P-300 | D-305 | R-327 |
| ADW02698 | T-220 | S-232 | D-243 | H-246 | I-294 | R-298 | P-299 | A-304 | Q-326 |
| ABC33525 | V-219 | E-231 | D-243 | N-246 | V-291 | T-295 | R-296 | Q-301 | A-310 |
| ADB34282 | V-222 | S-234 | D-245 | I-248 | L-292 | F-296 | R-297 | A-302 | G-316 |
| AAM23648 | T-222 | K-234 | D-245 | A-248 | V-296 | T-300 | R-301 | Y-306 | N-316 |
| CCH86028 | T-229 | — | D-252 | Q-255 | V-302 | F-306 | R-307 | A-312 | Q-330 |
| ADV67544 | V-227 | R-239 | D-250 | F-253 | V-298 | S-302 | R-303 | A-308 | P-314 |
| ACZ89862 | T-232 | — | D-250 | Q-253 | V-300 | H-304 | P-305 | A-310 | G-325 |
| ADH66252 | A-221 | — | D-244 | R-247 | V-294 | S-298 | P-299 | A-304 | E-327 |
| ADV80605 | T-219 | K-231 | D-242 | A-245 | V-293 | T-297 | R-298 | Y-303 | N-313 |
| ADW05507 | T-233 | — | D-256 | G-259 | I-307 | T-311 | P-312 | T-317 | H-343 |
| BAA86923 | V-217 | — | D-235 | H-238 | V-281 | A-285 | P-286 | P-291 | P-302 |
| AAN05438 | V-217 | — | D-235 | H-238 | V-281 | A-285 | P-286 | P-291 | P-302 |
| CAC16438 | T-233 | — | D-256 | A-259 | V-306 | T-310 | P-311 | E-316 | R-341 |
| CAJ89567 | T-233 | — | D-256 | A-259 | V-306 | T-310 | P-311 | E-316 | R-341 |
| AFD27167 | V-238 | A-250 | D-261 | A-264 | V-309 | S-313 | R-314 | E-319 | P-325 |
| BAG20044 | T-240 | — | D-263 | A-266 | I-313 | S-317 | P-318 | A-323 | R-337 |
| ACU71435 | T-223 | — | D-246 | H-249 | V-296 | R-300 | S-301 | G-306 | R-323 |
| ADG87563 | T-254 | — | D-275 | A-278 | V-325 | S-329 | P-330 | A-335 | G-350 |
| ABI35984 | V-217 | — | D-235 | H-238 | V-281 | A-285 | P-286 | P-291 | P-302 |
| CAB42553 | V-217 | — | D-235 | H-238 | V-281 | A-285 | P-286 | P-291 | P-302 |
| 1UG6 | V-217 | — | D-235 | H-238 | V-281 | A-285 | P-286 | P-291 | P-302 |
| CAA91220 | T-222 | K-234 | D-245 | A-248 | V-296 | T-300 | R-301 | Y-306 | N-316 |
| AFK85369 | T-218 | R-230 | D-241 | S-244 | I-292 | S-296 | R-297 | Y-302 | G-312 |
| AEN10177 | T-234 | — | D-257 | G-260 | I-308 | T-312 | P-313 | T-318 | H-344 |
| AEM87460 | S-220 | — | D-243 | A-246 | I-293 | T-297 | P-298 | A-303 | Q-330 |
| AAZ81839 | T-220 | N-232 | D-243 | Q-246 | V-294 | T-298 | R-299 | D-304 | H-315 |
| ADU50085 | T-242 | A-254 | D-266 | M-269 | V-316 | N-320 | G-321 | H-326 | D-351 |
| AEK47062 | T-233 | — | D-256 | G-259 | V-306 | F-310 | G-311 | G-316 | R-333 |
| ABD68852 | A-232 | S-244 | D-255 | R-258 | V-303 | F-307 | P-308 | D-313 | S-327 |
| ACL38401 | T-221 | G-233 | D-245 | F-248 | V-295 | H-299 | G-300 | K-305 | D-337 |
| ABS05424 | T-238 | — | D-261 | Q-264 | V-311 | S-315 | P-316 | A-321 | D-347 |
| AEK43773 | T-215 | — | D-239 | Q-242 | V-289 | R-293 | D-294 | G-299 | D-317 |
| AEV87561 | A-207 | — | D-230 | A-233 | V-280 | F-284 | G-285 | G-290 | R-307 |
| ACZ86244 | S-229 | — | D-252 | Q-255 | I-300 | S-304 | R-305 | G-310 | D-334 |
| AEB46173 | T-220 | — | D-243 | A-246 | V-293 | S-297 | T-298 | A-303 | Q-329 |
| BAG21567 | T-235 | — | D-258 | G-261 | V-309 | T-313 | P-314 | T-319 | H-345 |
| ABX05041 | T-220 | S-232 | D-243 | F-246 | V-297 | S-301 | R-302 | D-307 | S-318 |
| AFR09943 | T-241 | S-253 | E-264 | H-267 | L-314 | R-318 | P-319 | D-324 | P-344 |
| ACZ89285 | T-228 | S-240 | D-251 | V-254 | V-301 | Y-305 | P-306 | A-311 | A-333 |
| ACV58907 | T-217 | N-229 | D-240 | Q-243 | V-291 | T-295 | R-296 | D-301 | H-312 |
| ADG89462 | T-211 | S-223 | D-234 | H-237 | I-279 | T-283 | P-284 | G-289 | E-301 |
| ADU09756 | T-238 | T-250 | D-261 | L-264 | V-311 | Y-315 | R-316 | D-321 | R-343 |
| ACZ90607 | T-211 | — | D-234 | H-237 | I-280 | N-284 | P-285 | A-290 | G-302 |
| ADG73989 | T-227 | — | D-250 | A-253 | V-300 | S-304 | T-305 | H-310 | N-338 |
| AEG45154 | T-251 | S-263 | D-275 | F-278 | V-325 | N-329 | G-330 | A-335 | D-373 |
| AEJ43907 | T-217 | S-229 | D-240 | Q-243 | V-291 | T-295 | R-296 | D-301 | H-312 |
| ACZ20790 | T-236 | T-248 | D-260 | F-263 | V-314 | H-318 | G-319 | K-324 | D-351 |
| CCB77455 | A-225 | A-237 | D-248 | H-251 | I-297 | N-301 | P-302 | A-307 | P-320 |
| ADL46625 | T-235 | — | D-258 | A-261 | V-308 | F-312 | G-313 | G-318 | R-335 |
| ADU10772 | T-235 | — | D-258 | A-261 | V-308 | F-312 | G-313 | G-318 | R-335 |
| ADD45899 | G-222 | — | D-245 | Q-248 | V-295 | S-299 | R-300 | G-305 | H-329 |
| CCA53920 | T-234 | — | D-257 | G-260 | V-307 | R-311 | S-312 | G-317 | R-334 |
| BAJ28512 | S-234 | — | E-257 | G-260 | L-308 | N-312 | P-313 | A-318 | D-348 |
| ACL70277 | T-220 | — | D-243 | I-246 | I-293 | S-297 | R-298 | H-303 | V-314 |
| CBG72797 | S-228 | — | D-251 | A-254 | L-301 | T-305 | P-306 | A-311 | D-337 |
| AEW05616 | T-220 | Q-232 | D-243 | A-246 | V-294 | T-298 | R-299 | D-304 | Y-315 |
| AEB46623 | A-217 | G-229 | E-240 | H-243 | V-284 | N-288 | P-289 | A-294 | P-307 |
| 1GNX | T-233 | — | D-256 | A-259 | V-306 | S-310 | P-311 | E-316 | R-341 |
| ACV76621 | T-223 | — | D-247 | G-250 | I-297 | S-301 | T-302 | R-307 | D-333 |
| ACZ31628 | T-252 | — | D-276 | F-279 | V-326 | N-330 | G-331 | G-336 | E-371 |
| ADH67953 | T-219 | S-231 | R-242 | H-245 | V-292 | R-296 | P-297 | D-302 | R-324 |
| ADB34290 | T-234 | — | D-257 | G-260 | I-307 | F-311 | S-312 | G-317 | R-334 |
| ADU09106 | A-246 | T-258 | D-269 | H-272 | V-313 | N-317 | P-318 | A-323 | P-336 |
| CAA82733 | T-233 | — | D-256 | A-259 | V-306 | S-310 | P-311 | E-316 | R-341 |
| CBG67455 | T-250 | — | D-273 | A-276 | V-323 | S-327 | P-328 | E-333 | R-358 |
| CAN00920 | T-240 | — | G-261 | G-264 | V-311 | N-315 | T-316 | M-321 | D-347 |
| ADX71280 | T-221 | — | D-245 | F-248 | V-295 | H-299 | G-300 | K-305 | D-335 |
| ACQ81085 | T-235 | — | D-259 | Q-262 | V-308 | T-312 | A-313 | A-318 | D-356 |
| 3AHX | T-220 | — | D-243 | N-246 | I-293 | T-297 | R-298 | N-303 | S-313 |
| ACU35632 | T-232 | — | D-255 | G-258 | I-305 | T-309 | S-310 | G-315 | R-332 |
| AEV86556 | T-221 | S-233 | D-244 | I-247 | V-294 | Y-298 | R-299 | D-304 | A-326 |
| AEG45006 | T-231 | — | D-255 | A-258 | V-305 | S-309 | T-310 | K-315 | D-341 |
| BAG18801 | T-235 | — | D-258 | G-261 | V-308 | R-312 | G-313 | G-318 | R-335 |
| ADI03707 | T-230 | S-242 | E-253 | H-256 | L-303 | R-307 | P-308 | D-313 | D-335 |
| CAQ00266 | T-240 | — | D-261 | G-264 | V-311 | N-315 | T-316 | M-321 | D-347 |
| ADD43929 | T-220 | S-232 | D-243 | H-246 | V-290 | A-294 | P-295 | A-300 | E-312 |
| ADJ49823 | A-217 | — | D-240 | H-243 | V-292 | A-296 | P-297 | P-302 | R-318 |
| ABX05062 | T-222 | — | D-245 | G-248 | I-297 | F-301 | P-302 | A-307 | V-317 |
| ADL48215 | A-222 | T-234 | D-245 | H-248 | V-289 | N-293 | P-294 | A-299 | P-312 |
| ACU70272 | A-223 | A-235 | D-246 | H-249 | V-295 | N-299 | P-300 | A-305 | P-318 |
| ADG88606 | C-229 | — | D-252 | Q-255 | I-302 | S-306 | R-307 | G-312 | H-336 |
| AEW47954 | T-222 | S-234 | I-245 | N-248 | L-293 | T-297 | P-298 | A-303 | R-314 |
| ACD20223 | V-245 | S-257 | E-268 | H-271 | I-316 | F-320 | R-321 | S-326 | P-336 |
| AFC28171 | T-219 | S-231 | D-242 | T-245 | I-293 | T-297 | R-298 | A-303 | H-314 |
| ADW03239 | T-234 | — | D-257 | G-260 | V-307 | R-311 | G-312 | G-317 | R-334 |
| CBT74727 | T-223 | T-235 | D-247 | W-250 | V-297 | H-301 | D-302 | G-307 | E-333 |
| CAM04686 | A-223 | — | D-246 | H-249 | V-297 | A-301 | P-302 | P-307 | R-323 |
| ACZ20966 | T-226 | — | D-250 | A-253 | V-300 | S-304 | T-305 | H-310 | D-336 |
| AEV38153 | V-237 | A-249 | E-260 | Q-263 | I-308 | R-312 | R-313 | D-318 | R-329 |
| BAJ26623 | A-219 | A-231 | D-242 | H-245 | V-292 | N-296 | P-297 | A-302 | P-314 |
| BAL91665 | A-207 | — | D-230 | G-233 | V-280 | F-284 | G-285 | G-290 | R-307 |
| ACV09397 | T-224 | — | D-248 | A-251 | V-298 | S-302 | T-303 | E-308 | D-334 |
| ADG20157 | V-221 | D-232 | Y-244 | F-247 | I-294 | T-298 | R-299 | A-304 | G-312 |
| ADC61565 | A-218 | — | D-241 | I-244 | V-289 | S-293 | R-294 | A-299 | D-307 |
| ACM66669 | T-223 | G-235 | D-247 | W-250 | V-297 | H-301 | D-302 | G-307 | E-333 |
| ABD68843 | V-227 | D-238 | D-250 | G-253 | I-298 | M-302 | R-303 | A-308 | S-317 |
| ABS61373 | T-220 | R-232 | D-243 | I-246 | V-291 | T-295 | T-297 | D-302 | Y-311 |
| CBA30283 | V-235 | D-246 | Y-258 | F-261 | I-303 | T-307 | R-308 | T-313 | N-322 |
| AAA22266 | A-220 | T-232 | N-243 | S-246 | I-293 | T-297 | S-298 | Y-303 | A-316 |
| CAA42814 | A-220 | K-232 | F-243 | A-246 | F-293 | S-297 | S-298 | Y-303 | S-316 |
| ABN51453 | A-243 | K-255 | F-266 | A-269 | F-316 | S-320 | S-321 | Y-326 | S-339 |
| ACZ00292 | A-217 | S-229 | D-241 | M-244 | V-289 | Q-293 | P-294 | A-299 | P-312 |
| AEI12946 | T-227 | — | D-250 | A-253 | V-300 | A-304 | T-305 | H-310 | Q-338 |
| ABS15474 | V-236 | D-248 | D-259 | Q-262 | V-307 | R-311 | R-312 | A-317 | R-328 |
| ADL51094 | T-220 | D-232 | D-243 | N-246 | I-293 | T-297 | R-298 | N-303 | S-313 |
| AFG34202 | T-220 | K-232 | D-243 | V-246 | V-291 | T-295 | T-297 | D-302 | Y-311 |
| AAN60220 | T-220 | K-232 | D-243 | V-246 | V-291 | T-295 | T-297 | D-302 | Y-311 |

| Corresp. | Sequence Name | | | | | | | |
|---|---|---|---|---|---|---|---|---|
| Sequence Pos. | S317 | M325 | N326 | N332 | E365 | Q366 | I378 | Y399 |
| SEQ ID NO. | S-317 | M-325 | N-326 | N-332 | E-365 | Q-366 | I-378 | Y-399 |
| 379/380 | | | | | | | | |
| SEQ ID NO. 378 | R-315 | M-323 | N-324 | D-330 | D-363 | D-364 | Y-376 | F-397 |
| US8101393-0094 | S-317 | M-325 | N-326 | N-332 | E-365 | Q-366 | I-378 | Y-399 |
| US8101393-0388 | S-317 | M-325 | N-326 | N-332 | E-365 | Q-366 | I-378 | Y-399 |
| US8101393-0172 | — | M-322 | N-323 | D-329 | D-362 | D-363 | Y-375 | F-396 |
| JP2011205992- | — | A-323 | D-324 | E-330 | E-363 | D-364 | F-376 | F-397 |
| 0018 | | | | | | | | |
| JP2011205992- | — | M-320 | D-321 | E-327 | D-360 | D-361 | F-373 | F-394 |
| 0023 | | | | | | | | |
| JP2011205992- | — | M-325 | G-326 | R-332 | P-365 | E-366 | L-378 | F-399 |
| 0022 | | | | | | | | |
| US20110214199- | — | M-324 | D-325 | T-331 | P-364 | D-365 | L-377 | F-398 |
| 58656 | | | | | | | | |
| US20110214199- | — | M-340 | D-341 | G-347 | P-380 | D-381 | L-393 | F-414 |
| 62406 | | | | | | | | |
| US20110214199- | — | M-328 | D-329 | P-335 | E-368 | D-369 | L-381 | F-402 |
| 47919 | | | | | | | | |
| US20110214199- | P-325 | M-333 | G-334 | D-340 | P-373 | E-374 | L-386 | M-407 |
| 60662 | | | | | | | | |
| US20110214199- | T-332 | M-339 | G-340 | T-346 | A-379 | D-380 | L-392 | F-413 |
| 36660 | | | | | | | | |
| US20110214199- | T-346 | M-353 | G-354 | T-360 | P-393 | E-394 | L-406 | Y-427 |
| 17908 | | | | | | | | |
| US20110214199- | — | M-310 | G-311 | E-317 | G-349 | E-350 | L-362 | F-383 |
| 25308 | | | | | | | | |
| US20110214199- | — | M-310 | G-311 | E-317 | G-349 | E-350 | L-362 | F-383 |
| 64004 | | | | | | | | |
| US20110214199- | P-352 | M-359 | G-360 | T-366 | V-398 | D-399 | L-411 | F-432 |
| 25023 | | | | | | | | |
| US20110214199- | — | M-310 | G-311 | E-317 | G-349 | E-350 | L-362 | F-383 |
| 52644 | | | | | | | | |
| US20110214199- | P-343 | M-350 | G-351 | T-357 | A-390 | D-391 | L-403 | Y-424 |
| 13862 | | | | | | | | |
| US20110214199- | G-319 | M-326 | G-327 | E-333 | E-366 | D-367 | I-379 | F-400 |
| 29446 | | | | | | | | |
| US20110214199- | — | M-310 | G-311 | E-317 | G-349 | E-350 | L-362 | F-383 |
| 5988 | | | | | | | | |
| US20110214199- | — | M-310 | G-311 | E-317 | G-349 | E-350 | L-362 | F-383 |
| 23246 | | | | | | | | |
| US20110214199- | A-315 | M-323 | G-324 | Q-330 | R-363 | D-364 | L-376 | Y-397 |
| 12000 | | | | | | | | |
| US20110214199- | Q-313 | M-321 | G-322 | Q-328 | E-359 | N-360 | L-372 | F-393 |
| 56949 | | | | | | | | |
| US8101393-0272 | P-334 | M-342 | D-343 | D-349 | P-382 | S-383 | L-395 | F-416 |
| US8101393-0014 | — | M-333 | G-334 | D-340 | A-373 | E-374 | L-386 | F-407 |
| US8101393-0398 | R-346 | M-353 | G-354 | P-360 | V-392 | D-393 | F-405 | F-426 |
| US8101393-0492 | R-346 | M-353 | G-354 | P-360 | V-392 | D-393 | F-405 | F-426 |
| US8101393-0400 | R-346 | M-353 | D-354 | P-360 | V-392 | D-393 | F-405 | F-426 |
| US8101393-0266 | A-345 | M-352 | G-353 | S-359 | P-392 | D-393 | V-405 | F-426 |
| US8101393-0366 | — | M-308 | G-309 | E-315 | G-347 | E-348 | L-360 | F-381 |
| US8101393-0342 | — | M-332 | G-333 | E-339 | P-372 | N-373 | L-385 | F-406 |
| US8101393-0356 | — | R-322 | D-323 | Q-329 | P-362 | D-363 | L-375 | F-396 |
| US8101393-0320 | L-318 | M-326 | G-327 | E-333 | S-366 | N-367 | L-379 | Y-400 |
| US7314974-19451 | — | M-326 | D-327 | P-333 | E-366 | D-367 | L-379 | F-400 |
| US7314974-8251 | P-327 | M-334 | G-335 | T-341 | V-373 | D-374 | L-386 | F-407 |
| US7630836-9340 | — | M-324 | D-325 | T-331 | P-364 | D-365 | L-377 | F-398 |
| US7630836-12787 | T-346 | M-353 | G-354 | T-360 | P-393 | E-394 | L-406 | Y-427 |
| US20090220480- | — | M-328 | G-329 | S-335 | A-368 | N-369 | L-381 | F-402 |
| 0018 | | | | | | | | |
| US20120034253- | — | M-310 | G-311 | E-317 | G-349 | E-350 | L-362 | F-383 |
| 0080 | | | | | | | | |
| US20120034253- | — | M-310 | G-311 | E-317 | G-349 | E-350 | L-362 | F-383 |
| 0077 | | | | | | | | |
| US20120034253- | — | M-310 | G-311 | E-317 | G-349 | E-350 | L-362 | F-383 |
| 0078 | | | | | | | | |
| WO2012016960- | P-351 | M-359 | N-360 | A-366 | A-399 | D-400 | V-412 | F-433 |
| 11369 | | | | | | | | |
| US20120015408- | G-319 | M-326 | G-327 | E-333 | E-366 | D-367 | I-379 | F-400 |
| 0002 | | | | | | | | |
| US20120015408- | G-319 | M-326 | G-327 | E-333 | E-366 | D-367 | I-379 | F-400 |
| 0004 | | | | | | | | |
| US20120015408- | G-319 | M-326 | G-327 | E-333 | E-366 | D-367 | I-379 | F-400 |
| 0006 | | | | | | | | |
| US20120015408- | G-319 | M-326 | G-327 | E-333 | E-366 | D-367 | I-379 | F-400 |
| 0008 | | | | | | | | |
| US20120015408- | G-319 | M-326 | G-327 | E-333 | E-366 | D-367 | I-379 | F-400 |
| 0010 | | | | | | | | |
| US20120015408- | G-319 | M-326 | G-327 | E-333 | E-366 | D-367 | I-379 | F-400 |
| 0012 | | | | | | | | |
| US20120015408- | G-319 | M-326 | G-327 | E-333 | E-366 | D-367 | I-379 | F-400 |
| 0014 | | | | | | | | |
| US20120015408- | G-319 | M-326 | G-327 | E-333 | E-366 | D-367 | I-379 | F-400 |
| 0016 | | | | | | | | |
| US20120015408- | G-319 | M-326 | G-327 | E-333 | E-366 | D-367 | I-379 | F-400 |
| 0018 | | | | | | | | |
| US20120015408- | G-319 | M-326 | G-327 | E-333 | E-366 | D-367 | I-379 | F-400 |
| 0020 | | | | | | | | |
| US20120015408- | G-319 | M-326 | G-327 | E-333 | E-366 | D-367 | I-379 | F-400 |
| 0022 | | | | | | | | |
| US20120015408- | G-319 | M-326 | G-327 | E-333 | E-366 | D-367 | I-379 | F-400 |
| 0026 | | | | | | | | |
| US20120015408- | G-319 | M-326 | G-327 | E-333 | E-366 | D-367 | I-379 | F-400 |
| 0028 | | | | | | | | |
| US20120015408- | G-319 | M-326 | G-327 | E-333 | E-366 | D-367 | I-379 | F-400 |
| 0030 | | | | | | | | |
| US20120015408- | G-319 | M-326 | G-327 | E-333 | E-366 | D-367 | I-379 | F-400 |
| 0032 | | | | | | | | |
| US20120015408- | G-319 | M-326 | G-327 | E-333 | E-366 | D-367 | I-379 | F-400 |
| 0034 | | | | | | | | |
| US20120015408- | G-319 | M-326 | G-327 | E-333 | E-366 | D-367 | I-379 | F-400 |
| 0036 | | | | | | | | |
| US20120015408- | G-319 | M-326 | G-327 | E-333 | E-366 | D-367 | I-379 | F-400 |
| 0038 | | | | | | | | |
| US20120015408- | G-319 | M-326 | G-327 | E-333 | E-366 | D-367 | I-379 | F-400 |
| 0040 | | | | | | | | |
| US20120015408- | G-319 | M-326 | G-327 | E-333 | E-366 | D-367 | I-379 | F-400 |
| 0042 | | | | | | | | |
| US20120015408- | G-319 | M-326 | G-327 | E-333 | E-366 | D-367 | I-379 | F-400 |
| 0044 | | | | | | | | |
| US20120015408- | G-319 | M-326 | G-327 | E-333 | E-366 | D-367 | I-379 | F-400 |
| 0046 | | | | | | | | |
| US20120015408- | G-319 | M-326 | G-327 | E-333 | E-366 | D-367 | I-379 | F-400 |
| 0048 | | | | | | | | |
| US20120015408- | G-319 | M-326 | G-327 | E-333 | E-366 | D-367 | I-379 | F-400 |
| 0050 | | | | | | | | |
| US20120015408- | G-319 | M-326 | G-327 | E-333 | E-366 | D-367 | I-379 | F-400 |
| 0052 | | | | | | | | |
| US20120015408- | G-319 | M-326 | G-327 | E-333 | E-366 | D-367 | I-379 | F-400 |
| 0054 | | | | | | | | |
| US20120015408- | G-319 | M-326 | G-327 | E-333 | E-366 | D-367 | I-379 | F-400 |
| 0056 | | | | | | | | |
| US20120015408- | G-319 | M-326 | G-327 | E-333 | E-366 | D-367 | I-379 | F-400 |
| 0058 | | | | | | | | |
| US20120015408- | G-319 | M-326 | G-327 | E-333 | E-366 | D-367 | I-379 | F-400 |
| 0060 | | | | | | | | |
| US20120015408- | G-319 | M-326 | G-327 | E-333 | E-366 | D-367 | I-379 | F-400 |
| 0062 | | | | | | | | |
| US20120015408- | G-319 | M-326 | G-327 | E-333 | E-366 | D-367 | I-379 | F-400 |
| 0064 | | | | | | | | |
| US20120015408- | G-319 | M-326 | G-327 | E-333 | E-366 | D-367 | I-379 | F-400 |
| 0066 | | | | | | | | |
| US20120015408- | G-319 | M-326 | G-327 | E-333 | E-366 | D-367 | I-379 | F-400 |
| 0068 | | | | | | | | |
| US20120015408- | G-319 | M-326 | G-327 | E-333 | E-366 | D-367 | I-379 | F-400 |
| 0070 | | | | | | | | |
| US20120015408- | G-319 | M-326 | G-327 | E-333 | E-366 | D-367 | I-379 | F-400 |
| 0072 | | | | | | | | |
| US20120015408- | G-319 | M-326 | G-327 | E-333 | E-366 | D-367 | I-379 | F-400 |
| 0074 | | | | | | | | |
| US20120015408- | G-319 | M-326 | G-327 | E-333 | E-366 | D-367 | I-379 | F-400 |
| 0076 | | | | | | | | |
| US20120015408- | G-319 | M-326 | G-327 | E-333 | E-366 | D-367 | I-379 | F-400 |
| 0078 | | | | | | | | |
| US20120015408- | G-319 | M-326 | G-327 | E-333 | E-366 | D-367 | I-379 | F-400 |
| 0080 | | | | | | | | |
| US20120015408- | G-319 | M-326 | G-327 | E-333 | E-366 | D-367 | I-379 | F-400 |
| 0082 | | | | | | | | |
| US20120015408- | G-319 | M-326 | G-327 | E-333 | E-366 | D-367 | I-379 | F-400 |
| 0086 | | | | | | | | |
| US20120015408- | G-319 | M-326 | G-327 | E-333 | E-366 | D-367 | I-379 | F-400 |
| 0084 | | | | | | | | |
| US20120015408- | G-319 | M-326 | G-327 | E-333 | E-366 | D-367 | I-379 | F-400 |
| 0088 | | | | | | | | |
| US20120015408- | G-319 | M-326 | G-327 | E-333 | E-366 | D-367 | I-379 | F-400 |
| 0090 | | | | | | | | |
| US20120015408- | G-319 | M-326 | G-327 | E-333 | E-366 | D-367 | I-379 | F-400 |
| 0092 | | | | | | | | |
| US20120015408- | G-319 | M-326 | G-327 | E-333 | E-366 | D-367 | I-379 | F-400 |
| 0094 | | | | | | | | |
| US20120015408- | G-319 | M-326 | G-327 | E-333 | E-366 | D-367 | I-379 | F-400 |
| 0096 | | | | | | | | |
| US20120015408- | G-319 | M-326 | G-327 | E-333 | E-366 | D-367 | I-379 | F-400 |
| 0098 | | | | | | | | |
| US20120015408- | G-319 | M-326 | G-327 | E-333 | E-366 | D-367 | I-379 | F-400 |
| 0100 | | | | | | | | |
| US20120015408- | G-319 | M-326 | G-327 | E-333 | E-366 | D-367 | I-379 | F-400 |
| 0102 | | | | | | | | |
| US20120015408- | G-319 | M-326 | G-327 | E-333 | E-366 | D-367 | I-379 | F-400 |
| 0104 | | | | | | | | |
| US20120015408- | G-319 | M-326 | G-327 | E-333 | E-366 | D-367 | I-379 | F-400 |
| 0106 | | | | | | | | |
| US20120015408- | G-319 | M-326 | G-327 | E-333 | E-366 | D-367 | I-379 | F-400 |
| 0108 | | | | | | | | |
| US20120015408- | G-319 | M-326 | G-327 | E-333 | E-366 | D-367 | I-379 | F-400 |
| 0110 | | | | | | | | |
| US20120015408- | G-319 | M-326 | G-327 | E-333 | E-366 | D-367 | I-379 | F-400 |
| 0112 | | | | | | | | |
| US20120015408- | G-319 | M-326 | G-327 | E-333 | E-366 | D-367 | I-379 | F-400 |
| 0114 | | | | | | | | |
| US20120015408- | G-319 | M-326 | G-327 | E-333 | E-366 | D-367 | I-379 | F-400 |
| 0116 | | | | | | | | |
| US20120015408- | G-319 | M-326 | G-327 | E-333 | E-366 | D-367 | I-379 | F-400 |
| 0118 | | | | | | | | |
| US20120015408- | G-319 | M-326 | G-327 | E-333 | E-366 | D-367 | I-379 | F-400 |
| 0120 | | | | | | | | |
| US20120015408- | G-319 | M-326 | G-327 | E-333 | E-366 | D-367 | I-379 | F-400 |
| 0122 | | | | | | | | |
| US20120015408- | G-319 | M-326 | G-327 | E-333 | E-366 | D-367 | I-379 | F-400 |
| 0124 | | | | | | | | |
| US8202716-0464 | G-318 | M-325 | D-326 | D-332 | — | D-365 | L-377 | Y-398 |
| US20110262988- | L-318 | M-326 | G-327 | E-333 | S-366 | N-367 | L-379 | Y-400 |
| 0002 | | | | | | | | |
| US20110262988- | L-341 | M-349 | G-350 | E-356 | S-389 | N-390 | L-402 | Y-423 |
| 0003 | | | | | | | | |
| US20110151538- | L-370 | M-378 | G-379 | E-385 | S-418 | N-419 | L-431 | Y-452 |
| 0130 | | | | | | | | |
| US20100003234- | P-318 | M-326 | G-327 | Q-333 | S-366 | N-367 | L-379 | F-400 |
| 0048 | | | | | | | | |
| US6184018-0012 | L-318 | M-326 | G-327 | E-333 | S-366 | N-367 | L-379 | Y-400 |
| US6377893-0063 | L-313 | M-321 | G-322 | E-328 | S-361 | N-362 | L-374 | Y-395 |
| ACJ34717 | S-328 | M-336 | N-337 | N-343 | E-376 | D-377 | I-389 | Y-410 |
| ADD27066 | — | M-320 | D-321 | E-327 | D-360 | D-361 | F-373 | F-394 |
| ACZ42845 | — | M-329 | G-330 | Q-336 | V-369 | E-370 | Y-382 | F-403 |
| ABQ91969 | — | M-323 | D-324 | D-330 | P-363 | D-364 | I-376 | F-397 |
| ABU56651 | — | M-323 | D-324 | A-330 | S-363 | D-364 | I-376 | F-397 |
| AEY92801 | — | M-326 | D-327 | S-333 | P-366 | D-367 | L-379 | F-400 |
| CAD55382 | — | M-340 | D-341 | G-347 | P-380 | D-381 | L-393 | F-414 |
| AD073143 | — | M-332 | G-333 | D-339 | E-372 | D-373 | L-385 | F-406 |
| CCA60311 | P-334 | M-342 | D-343 | D-349 | P-382 | S-383 | L-395 | F-416 |
| ADI15206 | — | M-331 | G-332 | Q-338 | E-370 | G-371 | Y-383 | Y-404 |
| ADI12494 | — | M-326 | D-327 | T-333 | P-366 | D-367 | L-379 | F-400 |
| ACU35736 | R-333 | M-341 | E-342 | R-348 | D-381 | D-382 | I-394 | F-415 |
| BAC69512 | — | M-324 | D-325 | T-331 | P-364 | D-365 | L-377 | F-398 |
| AEN08263 | — | M-326 | D-327 | G-333 | P-366 | D-367 | L-379 | Y-400 |
| CCA53915 | — | M-350 | D-351 | G-357 | P-390 | D-391 | L-403 | Y-424 |
| CAJ88063 | — | M-326 | D-327 | G-333 | P-366 | D-367 | L-379 | Y-400 |
| ABF87202 | — | M-328 | D-329 | P-335 | E-368 | D-369 | L-381 | F-402 |
| BAJ30040 | — | M-323 | D-324 | P-330 | A-363 | D-364 | L-376 | F-397 |
| ACO44852 | — | M-317 | H-318 | Q-324 | H-357 | A-358 | Y-370 | F-391 |
| AFO59750 | R-327 | M-334 | G-335 | D-341 | R-374 | E-375 | L-387 | F-408 |
| BAJ31549 | P-360 | M-368 | G-369 | D-375 | P-408 | S-409 | L-421 | F-442 |
| ACY97307 | R-349 | M-356 | G-357 | E-363 | P-396 | D-397 | L-409 | F-430 |
| ACM06095 | D-320 | M-328 | G-329 | E-335 | A-368 | — | L-380 | F-401 |
| AEI64652 | — | M-328 | G-329 | P-335 | E-368 | D-369 | L-381 | F-402 |
| BAG17581 | — | M-340 | D-341 | A-347 | A-380 | D-381 | L-393 | F-414 |
| AEM83530 | — | M-326 | D-327 | T-333 | P-366 | D-367 | L-379 | F-400 |
| ADG89307 | — | L-341 | G-342 | E-348 | — | D-377 | L-389 | F-410 |
| ABG04991 | P-325 | M-333 | G-334 | D-340 | P-373 | E-374 | L-386 | M-407 |
| ABP54026 | R-347 | M-354 | D-355 | S-361 | V-393 | D-394 | F-406 | F-427 |
| ABV97405 | R-347 | M-354 | D-355 | P-361 | V-393 | E-394 | F-406 | F-427 |
| ADH60167 | G-316 | M-323 | G-324 | E-330 | E-363 | D-364 | I-376 | F-397 |
| ADV80493 | G-316 | M-323 | G-324 | E-330 | E-363 | D-364 | I-376 | F-397 |
| AEM77729 | G-316 | M-323 | G-324 | E-330 | E-363 | D-364 | I-376 | F-397 |
| ABK71329 | T-332 | M-339 | G-340 | T-346 | A-379 | D-380 | L-392 | F-413 |
| ACZ89864 | A-342 | M-350 | G-351 | N-357 | E-389 | D-390 | L-402 | L-423 |
| BAC72965 | T-346 | M-353 | G-354 | T-360 | P-393 | E-394 | L-406 | Y-427 |
| ADD25173 | G-317 | M-324 | G-325 | E-331 | E-364 | D-365 | I-377 | F-398 |
| ABF44291 | — | M-325 | G-326 | Q-332 | E-365 | D-366 | L-378 | F-399 |
| AEY93261 | — | M-341 | G-342 | G-348 | A-383 | D-384 | L-396 | Y-417 |
| 1NP2 | — | M-310 | G-311 | E-317 | G-349 | E-350 | L-362 | F-383 |
| AEG34643 | — | M-310 | G-311 | E-317 | G-349 | E-350 | L-362 | F-383 |
| AFR07907 | Q-333 | M-340 | G-341 | R-347 | S-379 | E-380 | Y-393 | F-414 |
| AEB43702 | R-346 | M-353 | D-354 | P-360 | I-392 | D-393 | F-405 | F-426 |
| BAL92882 | P-336 | M-344 | N-345 | A-351 | E-384 | N-385 | I-397 | F-418 |
| CCA59876 | A-345 | M-352 | G-353 | S-359 | P-392 | D-393 | V-405 | F-426 |
| ACU74192 | R-338 | M-346 | D-347 | S-353 | P-386 | D-387 | L-399 | F-420 |
| ABV96319 | P-325 | M-333 | G-334 | A-340 | G-385 | A-386 | L-398 | L-419 |
| AF053528 | P-346 | M-353 | G-354 | T-360 | P-393 | E-394 | V-406 | F-427 |
| AFH40090 | — | M-310 | G-311 | E-317 | G-349 | E-350 | L-362 | F-383 |
| ADL49193 | P-324 | M-332 | G-333 | A-339 | E-373 | Q-374 | L-386 | L-407 |
| AFE08200 | — | M-328 | D-329 | A-335 | A-368 | D-369 | L-381 | F-402 |
| AAN05441 | — | M-310 | G-311 | E-317 | G-349 | E-350 | L-362 | F-383 |
| CAJ90043 | — | M-335 | G-336 | D-342 | A-377 | D-378 | L-390 | Y-411 |
| AEY89575 | T-339 | M-346 | G-347 | T-353 | P-386 | D-387 | L-399 | Y-420 |
| AAF37730 | P-352 | M-359 | G-360 | T-366 | V-398 | D-399 | L-411 | F-432 |
| BAL98072 | — | M-330 | G-331 | Q-337 | A-370 | D-371 | L-383 | F-404 |
| AAN05440 | — | M-310 | G-311 | E-317 | G-349 | E-350 | L-362 | F-383 |
| AEM77895 | G-316 | M-323 | G-324 | E-330 | E-363 | D-364 | I-376 | F-397 |
| ADB34272 | G-341 | M-348 | G-349 | D-355 | A-388 | D-389 | L-401 | F-422 |
| ABW87307 | — | M-310 | G-311 | E-317 | G-349 | E-350 | L-362 | F-383 |
| AF322365 1 | — | M-310 | G-311 | E-317 | G-349 | E-350 | L-362 | F-383 |
| CAC10107 | P-343 | M-350 | G-351 | T-357 | A-390 | D-391 | L-403 | Y-424 |
| AEB47478 | P-325 | M-333 | G-334 | A-340 | G-375 | T-376 | L-388 | L-409 |
| ADL45220 | R-346 | M-353 | D-354 | P-360 | V-392 | D-393 | F-405 | F-426 |
| ABK51908 | W-347 | M-354 | G-355 | S-361 | D-393 | N-394 | I-406 | Y-427 |
| CAN94460 | — | M-333 | Q-334 | E-340 | K-373 | D-374 | L-386 | F-407 |
| AEF18219 | G-315 | M-322 | G-323 | E-329 | K-362 | D-363 | V-374 | F-395 |
| ADD39191 | P-357 | M-364 | G-365 | E-371 | A-404 | A-405 | V-417 | F-438 |
| ABP52811 | S-325 | M-333 | G-334 | A-340 | G-384 | V-385 | L-397 | L-418 |
| AEV88819 | P-351 | M-359 | N-360 | A-366 | A-399 | D-400 | V-412 | F-433 |
| AEY89570 | A-349 | M-356 | N-357 | S-363 | P-396 | E-397 | L-409 | F-430 |
| ADD01635 | G-316 | M-323 | G-324 | E-330 | E-363 | D-364 | I-376 | F-397 |
| CAB95278 | — | M-335 | N-336 | D-342 | A-377 | D-378 | L-390 | Y-411 |
| AEN13042 | — | M-335 | G-336 | H-342 | A-377 | D-378 | L-390 | Y-411 |
| ACY97750 | M-335 | M-342 | G-343 | A-349 | A-382 | D-383 | L-395 | F-416 |
| ADI10010 | T-335 | M-342 | G-343 | T-349 | A-382 | D-383 | L-395 | F-416 |
| CCB72805 | P-343 | M-350 | G-351 | S-357 | A-390 | S-391 | I-403 | F-424 |
| ACY14034 | L-337 | M-344 | G-345 | D-351 | A-384 | D-385 | V-397 | L-418 |
| AAZ55664 | — | M-336 | D-337 | E-343 | P-377 | D-378 | L-390 | F-411 |
| ADW02698 | — | M-335 | G-336 | E-342 | A-377 | D-378 | L-390 | Y-411 |
| ABC33525 | — | M-317 | G-318 | Q-324 | — | K-357 | L-369 | F-390 |
| ADB34282 | — | I-324 | D-325 | A-331 | A-364 | E-365 | L-377 | F-398 |
| AAM23648 | G-319 | M-326 | G-327 | E-333 | E-366 | D-367 | I-379 | F-400 |
| CCH86028 | P-334 | M-341 | G-342 | E-348 | A-381 | D-382 | L-394 | F-415 |
| ADV67544 | — | M-322 | N-323 | Q-329 | E-365 | H-366 | F-378 | F-399 |
| ACZ89862 | R-330 | M-338 | G-339 | F-345 | E-377 | D-378 | L-390 | L-411 |
| ADH66252 | Q-333 | M-340 | G-341 | T-347 | T-379 | E-380 | Y-393 | F-414 |
| ADV80605 | G-316 | M-323 | G-324 | E-330 | E-363 | D-364 | I-376 | F-397 |
| ADW05507 | P-349 | M-357 | D-358 | E-364 | P-397 | E-398 | L-410 | F-431 |
| BAA86923 | — | M-310 | G-311 | E-317 | G-349 | E-350 | L-362 | F-383 |
| AAN05438 | — | M-310 | G-311 | E-317 | G-349 | E-350 | L-362 | F-383 |
| CAC16438 | P-347 | M-354 | G-355 | T-361 | P-394 | E-395 | V-407 | F-428 |
| CAJ89567 | P-347 | M-354 | G-355 | T-361 | P-394 | A-395 | V-407 | F-428 |
| AFD27167 | — | M-333 | G-334 | Q-340 | E-373 | N-374 | L-386 | F-407 |
| BAG20044 | P-343 | M-351 | D-352 | D-358 | P-391 | E-392 | L-404 | F-425 |
| ACU71435 | F-327 | I-335 | D-336 | E-342 | A-375 | D-376 | I-388 | F-409 |
| ADG87563 | R-355 | M-363 | G-364 | D-370 | D-402 | G-403 | L-415 | F-436 |
| ABI35984 | — | M-310 | G-311 | E-317 | G-349 | E-350 | L-362 | F-383 |
| CAB42553 | — | M-310 | G-311 | E-317 | G-349 | E-350 | L-362 | F-383 |
| 1UG6 | — | M-310 | G-311 | E-317 | G-349 | E-350 | L-362 | F-383 |
| CAA91220 | G-319 | M-326 | G-327 | E-333 | E-366 | D-367 | I-379 | F-400 |
| AFK85369 | G-315 | M-322 | G-323 | E-329 | E-362 | D-363 | I-375 | F-396 |
| AEN10177 | A-350 | M-358 | D-359 | D-365 | P-398 | E-399 | L-411 | F-432 |
| AEM87460 | T-336 | M-343 | G-344 | T-350 | A-383 | E-384 | L-396 | F-417 |
| AAZ81839 | G-318 | M-325 | D-326 | D-332 | — | D-365 | L-377 | Y-398 |
| ADU50085 | P-357 | M-365 | D-366 | E-372 | E-408 | Q-409 | V-421 | F-442 |
| AEK47062 | F-337 | M-344 | G-345 | G-351 | S-384 | S-385 | F-397 | F-418 |
| ABD68852 | — | F-335 | G-336 | E-342 | P-375 | E-376 | L-388 | F-409 |
| ACL38401 | R-343 | M-351 | G-352 | E-358 | A-394 | T-395 | F-407 | L-428 |
| ABS05424 | L-353 | M-360 | G-361 | R-367 | A-400 | G-401 | L-412 | Y-433 |
| AEK43773 | D-323 | S-331 | G-332 | A-338 | D-371 | G-372 | L-384 | F-405 |
| AEV87561 | P-311 | M-318 | G-319 | D-325 | P-357 | D-358 | L-370 | F-391 |
| ACZ86244 | G-340 | M-347 | G-348 | S-354 | A-387 | D-388 | I-400 | F-421 |
| AEB46173 | Q-335 | M-342 | G-343 | V-349 | P-382 | D-383 | L-395 | F-416 |
| BAG21567 | A-351 | M-359 | N-360 | E-366 | A-399 | D-400 | L-412 | F-433 |
| ABX05041 | — | I-323 | G-324 | Q-330 | A-363 | H-364 | L-376 | F-397 |
| AFR09943 | — | M-353 | G-354 | S-360 | P-394 | D-395 | L-407 | F-428 |
| ACZ89285 | — | L-342 | G-343 | R-349 | — | R-378 | L-390 | F-411 |
| ACV58907 | G-315 | M-322 | D-323 | D-329 | — | D-362 | L-374 | Y-395 |
| ADG89462 | — | F-310 | G-311 | D-317 | Y-346 | D-347 | L-358 | F-377 |
| ADU09756 | — | L-352 | G-353 | Q-359 | R-389 | D-390 | V-402 | F-425 |
| ACZ90607 | — | F-311 | G-312 | D-318 | P-353 | D-354 | L-366 | Y-387 |
| ADG73989 | Q-344 | M-351 | G-352 | Q-358 | D-391 | D-392 | L-404 | F-425 |
| AEG45154 | S-379 | M-387 | G-388 | E-394 | P-430 | D-431 | I-443 | F-464 |
| AEJ43907 | G-315 | M-322 | D-323 | D-329 | — | D-362 | L-374 | Y-395 |
| ACZ20790 | P-357 | M-365 | D-366 | E-372 | P-408 | D-409 | Y-421 | F-442 |
| CCB77455 | — | F-329 | D-330 | E-336 | — | P-366 | L-377 | F-398 |
| ADL46625 | R-339 | M-346 | D-347 | E-353 | A-386 | D-387 | L-399 | F-420 |
| ADU10772 | R-339 | M-346 | D-347 | E-353 | A-386 | D-387 | L-399 | F-420 |
| ADD45899 | A-335 | M-342 | G-343 | D-349 | D-382 | D-383 | L-396 | F-417 |
| CCA53920 | H-338 | L-346 | G-347 | H-353 | A-386 | D-387 | L-399 | F-420 |
| BAJ28512 | A-354 | M-361 | G-362 | T-368 | P-400 | D-401 | V-413 | Y-434 |
| ACL70277 | M-317 | M-325 | G-326 | Q-332 | E-365 | E-366 | L-378 | Y-399 |
| CBG72797 | T-343 | M-350 | G-351 | T-357 | A-390 | D-391 | L-403 | Y-424 |
| AEW05616 | E-319 | M-326 | G-327 | E-333 | V-367 | D-368 | L-380 | Y-401 |
| AEB46623 | — | F-316 | D-317 | D-323 | A-358 | D-359 | L-371 | F-392 |
| 1GNX | P-347 | M-354 | G-355 | S-361 | P-394 | E-395 | V-407 | F-428 |
| ACV76621 | Q-339 | M-346 | G-347 | S-353 | E-386 | D-387 | L-399 | F-420 |
| ACZ31628 | S-377 | M-385 | G-386 | E-392 | E-428 | S-429 | I-442 | F-463 |
| ADH67953 | — | M-333 | D-334 | A-340 | E-374 | T-375 | L-387 | F-408 |
| ADB34290 | L-338 | M-345 | D-346 | E-352 | E-385 | N-386 | F-398 | L-419 |
| ADU09106 | — | F-345 | D-346 | D-352 | A-387 | D-388 | L-400 | F-421 |
| CAA82733 | P-347 | M-354 | G-355 | S-361 | P-394 | E-395 | V-407 | F-428 |
| CBG67455 | P-364 | M-371 | G-372 | S-378 | P-411 | A-412 | V-424 | F-445 |
| CAN00920 | Q-353 | M-360 | G-361 | Q-367 | V-400 | E-401 | L-416 | Q-437 |
| ADX71280 | P-341 | M-349 | G-350 | E-356 | E-392 | A-393 | F-405 | L-426 |
| ACQ81085 | D-361 | M-369 | D-370 | D-376 | S-412 | D-413 | V-425 | F-446 |
| 3AHX | A-315 | M-323 | G-324 | Q-330 | R-363 | D-364 | L-376 | Y-397 |
| ACU35632 | Y-336 | M-343 | D-344 | E-350 | P-384 | D-385 | L-397 | F-418 |
| AEV86556 | — | L-335 | G-336 | Q-342 | R-372 | E-373 | V-385 | F-407 |
| AEG45006 | Q-347 | M-354 | G-355 | A-361 | V-394 | D-395 | L-411 | F-432 |
| BAG18801 | R-339 | M-346 | D-347 | T-353 | A-385 | D-386 | L-398 | F-419 |
| ADI03707 | — | M-344 | G-345 | A-351 | P-385 | D-386 | L-398 | Y-419 |
| CAQ00266 | Q-353 | M-360 | G-361 | Q-367 | V-400 | D-401 | L-417 | Q-438 |
| ADD43929 | — | F-321 | D-322 | D-328 | D-363 | D-364 | L-376 | L-397 |
| ADJ49823 | P-324 | F-332 | G-333 | S-339 | V-370 | D-371 | F-383 | F-404 |
| ABX05062 | P-320 | R-328 | G-329 | A-335 | A-369 | D-370 | L-382 | Y-403 |
| ADL48215 | — | F-321 | D-322 | D-328 | A-363 | D-364 | L-376 | F-397 |
| ACU70272 | — | F-327 | D-328 | D-334 | V-368 | D-369 | L-381 | L-402 |
| ADG88606 | G-342 | M-349 | G-350 | S-356 | E-389 | D-390 | L-402 | F-423 |
| AEW47954 | — | M-323 | D-324 | D-330 | P-364 | D-365 | L-377 | F-398 |
| ACD20223 | — | M-345 | G-346 | D-352 | I-385 | D-386 | L-398 | F-419 |
| AFC28171 | G-317 | M-324 | D-325 | D-331 | — | D-364 | Y-376 | Y-397 |
| ADW03239 | R-338 | M-345 | D-346 | T-352 | A-384 | D-385 | L-397 | F-418 |
| CBT74727 | A-339 | M-347 | G-348 | E-354 | E-388 | E-389 | I-401 | F-422 |
| CAM04686 | P-329 | F-337 | G-338 | S-344 | S-377 | D-378 | F-390 | L-411 |
| ACZ20966 | Q-342 | M-349 | G-350 | A-356 | D-389 | D-390 | L-402 | F-423 |
| AEV38153 | L-331 | M-339 | G-340 | Q-346 | A-379 | D-380 | V-392 | F-413 |
| BAJ26623 | — | F-323 | G-324 | E-330 | — | D-360 | L-372 | Y-393 |
| BAL91665 | P-311 | L-318 | G-319 | D-325 | A-357 | G-358 | L-370 | F-391 |
| ACV09397 | M-340 | M-347 | G-348 | A-354 | P-387 | D-388 | L-400 | F-421 |
| ADG20157 | — | M-319 | D-320 | Q-326 | A-358 | N-359 | Y-371 | F-392 |
| ADC61565 | P-313 | M-321 | D-322 | E-328 | R-361 | D-362 | L-374 | C-395 |
| ACM66669 | A-339 | M-347 | G-348 | E-354 | E-388 | A-389 | I-401 | F-422 |
| ABD68843 | — | M-324 | G-325 | Q-331 | V-363 | D-364 | I-376 | F-397 |
| ABS61373 | S-313 | M-321 | G-322 | Q-328 | E-359 | N-360 | L-372 | F-393 |
| CBA30283 | — | M-329 | G-330 | Q-336 | V-368 | D-369 | M-381 | F-402 |
| AAA22266 | S-318 | I-326 | G-327 | E-333 | L-366 | D-367 | L-379 | M-400 |
| CAA42814 | L-318 | M-326 | G-327 | E-333 | S-366 | N-367 | L-379 | Y-400 |
| ABN51453 | L-341 | M-349 | G-350 | E-356 | S-389 | N-390 | L-402 | Y-423 |
| ACZ00292 | — | M-321 | G-322 | D-328 | G-362 | P-363 | L-376 | F-397 |
| AEI12946 | L-344 | M-351 | G-352 | E-358 | E-391 | D-392 | L-404 | F-425 |
| ABS15474 | Q-330 | V-338 | G-339 | R-345 | P-379 | D-380 | Y-392 | F-413 |
| ADL51094 | A-315 | M-323 | G-324 | Q-330 | R-363 | D-364 | L-376 | Y-397 |
| AFG34202 | Q-313 | M-321 | G-322 | Q-328 | E-359 | N-360 | L-372 | F-393 |
| AAN60220 | Q-313 | M-321 | G-322 | Q-328 | E-359 | N-360 | L-372 | F-393 |

TABLE 9C

| Corresp. | Sequence name | | | | | | | |
|---|---|---|---|---|---|---|---|---|
| Sequence Pos. | V400 | W401 | S402 | R410 | D414 | K415 | R416 | V420 |
| SEQ ID NO. 379/380 | V-400 | W-401 | S-402 | R-410 | D-414 | K-415 | R-416 | V-420 |
| SEQ ID NO. 378 | A-398 | W-399 | S-400 | A-408 | D-412 | K-413 | R-414 | F-418 |
| US8101393-0094 | V-400 | W-401 | S-402 | A-410 | D-414 | K-415 | R-416 | V-420 |
| US8101393-0388 | V-400 | W-401 | S-402 | A-410 | D-414 | K-415 | R-416 | V-420 |
| US8101393-0172 | A-397 | W-398 | S-399 | A-407 | D-411 | K-412 | R-413 | F-417 |
| JP2011205992-0018 | A-398 | W-399 | S-400 | A-408 | S-412 | M-413 | R-414 | C-418 |
| JP2011205992-0023 | A-395 | W-396 | S-397 | A-405 | A-409 | K-410 | R-411 | V-415 |
| JP2011205992-0022 | A-400 | W-401 | S-402 | S-410 | T-414 | K-415 | R-416 | H-420 |
| US20110214199-58656 | A-399 | W-400 | S-401 | A-409 | D-413 | K-414 | R-415 | V-419 |
| US20110214199-62406 | A-415 | W-416 | S-417 | A-425 | D-429 | K-430 | R-431 | V-435 |
| US20110214199-47919 | A-403 | W-404 | S-405 | A-413 | Q-417 | K-418 | R-419 | V-423 |
| US20110214199-60662 | V-408 | W-409 | S-410 | A-418 | S-422 | K-423 | R-424 | V-428 |
| US20110214199-36660 | V-414 | W-415 | S-416 | A-424 | A-428 | K-429 | R-430 | I-434 |
| US20110214199-17908 | L-428 | W-429 | S-430 | A-438 | G-442 | K-443 | R-444 | V-448 |
| US20110214199-25308 | V-384 | W-385 | S-386 | A-394 | T-398 | R-399 | R-400 | Y-404 |
| US20110214199-64004 | V-384 | W-385 | S-386 | A-394 | T-398 | R-399 | R-400 | Y-404 |
| US20110214199-25023 | A-433 | W-434 | S-435 | A-443 | G-447 | K-448 | R-449 | V-453 |
| US20110214199-52644 | V-384 | W-385 | S-386 | A-394 | T-398 | R-399 | R-400 | Y-404 |
| US20110214199-13862 | L-425 | W-426 | S-427 | A-435 | E-439 | K-440 | R-441 | V-445 |
| US20110214199-29446 | V-401 | W-402 | S-403 | A-411 | S-415 | K-416 | R-417 | V-421 |
| US20110214199-5988 | V-384 | W-385 | S-386 | A-394 | T-398 | R-399 | R-400 | Y-404 |
| US20110214199-23246 | V-384 | W-385 | S-386 | A-394 | T-398 | R-399 | R-400 | Y-404 |
| US20110214199-12000 | I-398 | W-399 | S-400 | A-408 | E-412 | K-413 | R-414 | V-418 |
| US20110214199-56949 | I-394 | W-395 | S-396 | A-404 | S-408 | K-409 | R-410 | I-414 |
| US8101393-0272 | L-417 | W-418 | S-419 | A-427 | S-431 | K-432 | R-433 | V-437 |
| US8101393-0014 | Q-408 | W-409 | S-410 | A-418 | T-422 | Q-423 | R-424 | V-428 |
| US8101393-0398 | A-427 | W-428 | S-429 | A-437 | T-441 | K-442 | R-443 | V-447 |
| US8101393-0492 | A-427 | W-428 | S-429 | A-437 | T-441 | K-442 | R-443 | V-447 |
| US8101393-0400 | A-427 | W-428 | S-429 | A-437 | T-441 | K-442 | R-443 | V-447 |
| US8101393-0266 | L-427 | W-428 | S-429 | A-437 | A-441 | K-442 | R-443 | V-447 |
| US8101393-0366 | A-382 | W-383 | S-384 | A-392 | T-396 | K-397 | R-398 | L-402 |
| US8101393-0342 | V-407 | W-408 | S-409 | A-417 | T-421 | Q-422 | R-423 | V-427 |
| US8101393-0356 | C-397 | W-398 | S-399 | A-407 | S-411 | S-412 | R-413 | A-417 |
| US8101393-0320 | L-401 | W-402 | S-403 | A-411 | N-415 | K-416 | R-417 | V-421 |
| US7314974-19451 | A-401 | W-402 | S-403 | A-411 | Q-415 | K-416 | R-417 | V-421 |
| US7314974-8251 | A-408 | W-409 | S-410 | A-418 | G-422 | K-423 | R-424 | V-428 |
| US7630836-9340 | A-399 | W-400 | S-401 | A-409 | D-413 | K-414 | R-415 | V-419 |
| US7630836-12787 | L-428 | W-429 | S-430 | A-438 | G-442 | K-443 | R-444 | V-448 |
| US20090220480-0018 | L-403 | W-404 | S-405 | A-413 | S-417 | K-418 | R-419 | F-423 |
| US20120034253-0080 | V-384 | W-385 | S-386 | A-394 | T-398 | R-399 | R-400 | Y-404 |
| US20120034253-0077 | V-384 | W-385 | S-386 | A-394 | T-398 | R-399 | R-400 | Y-404 |
| US20120034253-0078 | V-384 | W-385 | S-386 | A-394 | T-398 | R-399 | R-400 | Y-404 |
| WO2012016960-11369 | L-434 | W-435 | S-436 | A-444 | A-448 | K-449 | R-450 | V-454 |
| US20120015408-0002 | V-401 | W-402 | S-403 | A-411 | S-415 | K-416 | R-417 | V-421 |
| US20120015408-0004 | V-401 | W-402 | S-403 | A-411 | S-415 | K-416 | R-417 | V-421 |
| US20120015408-0006 | V-401 | W-402 | S-403 | A-411 | S-415 | K-416 | R-417 | V-421 |
| US20120015408-0008 | V-401 | W-402 | S-403 | A-411 | S-415 | K-416 | R-417 | V-421 |
| US20120015408-0010 | V-401 | W-402 | S-403 | A-411 | S-415 | K-416 | R-417 | V-421 |
| US20120015408-0012 | V-401 | W-402 | S-403 | A-411 | S-415 | K-416 | R-417 | V-421 |
| US20120015408-0014 | V-401 | W-402 | S-403 | A-411 | S-415 | K-416 | R-417 | V-421 |
| US20120015408-0016 | V-401 | W-402 | S-403 | A-411 | S-415 | K-416 | R-417 | V-421 |
| US20120015408-0018 | V-401 | W-402 | S-403 | A-411 | S-415 | K-416 | R-417 | V-421 |
| US20120015408-0020 | V-401 | W-402 | S-403 | A-411 | S-415 | K-416 | R-417 | V-421 |
| US20120015408-0022 | V-401 | W-402 | S-403 | A-411 | S-415 | K-416 | R-417 | V-421 |
| US20120015408-0026 | V-401 | W-402 | S-403 | A-411 | S-415 | K-416 | R-417 | V-421 |
| US20120015408-0028 | V-401 | W-402 | S-403 | A-411 | S-415 | K-416 | R-417 | V-421 |
| US20120015408-0030 | V-401 | W-402 | S-403 | A-411 | S-415 | K-416 | R-417 | V-421 |
| US20120015408-0032 | V-401 | W-402 | S-403 | A-411 | S-415 | K-416 | R-417 | V-421 |
| US20120015408-0034 | V-401 | W-402 | S-403 | A-411 | S-415 | K-416 | R-417 | V-421 |
| US20120015408-0036 | V-401 | W-402 | S-403 | A-411 | S-415 | K-416 | R-417 | V-421 |
| US20120015408-0038 | V-401 | W-402 | S-403 | A-411 | S-415 | K-416 | R-417 | V-421 |
| US20120015408-0040 | V-401 | W-402 | S-403 | A-411 | S-415 | K-416 | R-417 | V-421 |
| US20120015408-0042 | V-401 | W-402 | S-403 | A-411 | S-415 | K-416 | R-417 | V-421 |
| US20120015408-0044 | V-401 | W-402 | S-403 | A-411 | S-415 | K-416 | R-417 | V-421 |
| US20120015408-0046 | V-401 | W-402 | S-403 | A-411 | S-415 | K-416 | R-417 | V-421 |
| US20120015408-0048 | V-401 | W-402 | S-403 | A-411 | S-415 | K-416 | R-417 | V-421 |
| US20120015408-0050 | V-401 | W-402 | S-403 | A-411 | S-415 | K-416 | R-417 | V-421 |
| US20120015408-0052 | V-401 | W-402 | S-403 | A-411 | S-415 | K-416 | R-417 | V-421 |
| US20120015408-0054 | V-401 | W-402 | S-403 | A-411 | S-415 | K-416 | R-417 | V-421 |
| US20120015408-0056 | V-401 | W-402 | S-403 | A-411 | S-415 | K-416 | R-417 | V-421 |
| US20120015408-0058 | V-401 | W-402 | S-403 | A-411 | S-415 | K-416 | R-417 | V-421 |
| US20120015408-0060 | V-401 | W-402 | S-403 | A-411 | S-415 | K-416 | R-417 | V-421 |
| US20120015408-0062 | V-401 | W-402 | S-403 | A-411 | S-415 | K-416 | R-417 | V-421 |
| US20120015408-0064 | V-401 | W-402 | S-403 | A-411 | S-415 | K-416 | R-417 | V-421 |
| US20120015408-0066 | V-401 | W-402 | S-403 | A-411 | S-415 | K-416 | R-417 | V-421 |
| US20120015408-0068 | V-401 | W-402 | S-403 | A-411 | S-415 | K-416 | R-417 | V-421 |
| US20120015408-0070 | V-401 | W-402 | S-403 | A-411 | S-415 | K-416 | R-417 | V-421 |
| US20120015408-0072 | V-401 | W-402 | S-403 | A-411 | S-415 | K-416 | R-417 | V-421 |
| US20120015408-0074 | V-401 | W-402 | S-403 | A-411 | S-415 | K-416 | R-417 | V-421 |
| US20120015408-0076 | V-401 | W-402 | S-403 | A-411 | S-415 | K-416 | R-417 | V-421 |
| US20120015408-0078 | V-401 | W-402 | S-403 | A-411 | S-415 | K-416 | R-417 | V-421 |
| US20120015408-0080 | V-401 | W-402 | S-403 | A-411 | S-415 | K-416 | R-417 | V-421 |
| US20120015408-0082 | V-401 | W-402 | S-403 | A-411 | S-415 | K-416 | R-417 | V-421 |
| US20120015408-0086 | V-401 | W-402 | S-403 | A-411 | S-415 | K-416 | R-417 | V-421 |
| US20120015408-0084 | V-401 | W-402 | S-403 | A-411 | S-415 | K-416 | R-417 | V-421 |
| US20120015408-0088 | V-401 | W-402 | S-403 | A-411 | S-415 | K-416 | R-417 | V-421 |
| US20120015408-0090 | V-401 | W-402 | S-403 | A-411 | S-415 | K-416 | R-417 | V-421 |
| US20120015408-0092 | V-401 | W-402 | S-403 | A-411 | S-415 | K-416 | R-417 | V-421 |
| US20120015408-0094 | V-401 | W-402 | S-403 | A-411 | S-415 | K-416 | R-417 | V-421 |
| US20120015408-0096 | V-401 | W-402 | S-403 | A-411 | S-415 | K-416 | R-417 | V-421 |
| US20120015408-0098 | V-401 | W-402 | S-403 | A-411 | S-415 | K-416 | R-417 | V-421 |
| US20120015408-0100 | V-401 | W-402 | S-403 | A-411 | S-415 | K-416 | R-417 | V-421 |
| US20120015408-0102 | V-401 | W-402 | S-403 | A-411 | S-415 | K-416 | R-417 | V-421 |
| US20120015408-0104 | V-401 | W-402 | S-403 | A-411 | S-415 | K-416 | R-417 | V-421 |
| US20120015408-0106 | V-401 | W-402 | S-403 | A-411 | S-415 | K-416 | R-417 | V-421 |
| US20120015408-0108 | V-401 | W-402 | S-403 | A-411 | S-415 | K-416 | R-417 | V-421 |
| US20120015408-0110 | V-401 | W-402 | S-403 | A-411 | S-415 | K-416 | R-417 | V-421 |
| US20120015408-0112 | V-401 | W-402 | S-403 | A-411 | S-415 | K-416 | R-417 | V-421 |
| US20120015408-0114 | V-401 | W-402 | S-403 | A-411 | S-415 | K-416 | R-417 | V-421 |
| US20120015408-0116 | V-401 | W-402 | S-403 | A-411 | S-415 | K-416 | R-417 | V-421 |
| US20120015408-0118 | V-401 | W-402 | S-403 | A-411 | S-415 | K-416 | R-417 | V-421 |
| US20120015408-0120 | V-401 | W-402 | S-403 | A-411 | S-415 | K-416 | R-417 | V-421 |
| US20120015408-0122 | V-401 | W-402 | S-403 | A-411 | S-415 | K-416 | R-417 | V-421 |
| US20120015408-0124 | V-401 | W-402 | S-403 | A-411 | S-415 | K-416 | R-417 | V-421 |
| US8202716-0464 | V-399 | W-400 | S-401 | A-409 | T-413 | K-414 | R-415 | V-419 |
| US20110262988-0002 | L-401 | W-402 | S-403 | A-411 | N-415 | K-416 | R-417 | V-421 |
| US20110262988-0003 | L-424 | W-425 | S-426 | A-434 | N-438 | K-439 | R-440 | V-444 |
| US20110151538-0130 | L-453 | W-454 | S-455 | A-463 | N-467 | K-468 | R-469 | V-473 |
| US20100003234-0048 | V-401 | W-402 | T-403 | A-411 | S-415 | K-416 | R-417 | I-421 |
| US6184018-0012 | L-401 | W-402 | S-403 | A-411 | N-415 | K-416 | R-417 | V-421 |
| US6377893-0063 | L-396 | W-397 | S-398 | A-406 | N-410 | K-411 | R-412 | V-416 |
| ACJ34717 | V-411 | W-412 | S-413 | A-421 | D-425 | K-426 | R-427 | V-431 |
| ADD27066 | A-395 | W-396 | S-397 | A-405 | A-409 | K-410 | R-411 | V-415 |
| ACZ42845 | A-404 | W-405 | S-406 | A-414 | S-418 | K-419 | R-420 | Y-424 |
| ABQ91969 | V-398 | W-399 | S-400 | A-408 | S-412 | R-413 | R-414 | I-418 |
| ABTJ56651 | V-398 | W-399 | S-400 | A-408 | S-412 | R-413 | R-414 | V-418 |
| AEY92801 | A-401 | W-402 | S-403 | A-411 | D-415 | K-416 | R-417 | V-421 |
| CAD55382 | A-415 | W-416 | S-417 | A-425 | D-429 | K-430 | R-431 | V-435 |
| AD073143 | V-407 | W-408 | S-409 | D-417 | S-421 | Q-422 | R-423 | V-427 |
| CCA60311 | L-417 | W-418 | S-419 | A-427 | S-431 | K-432 | R-433 | V-437 |
| ADI15206 | A-405 | W-406 | S-407 | A-415 | S-419 | K-420 | R-421 | F-425 |
| ADI12494 | A-401 | W-402 | S-403 | A-411 | G-415 | K-416 | R-417 | V-421 |
| ACU35736 | A-416 | W-417 | S-418 | A-426 | A-430 | K-431 | R-432 | V-436 |
| BAC69512 | A-399 | W-400 | S-401 | A-409 | D-413 | K-414 | R-415 | V-419 |
| AEN08263 | A-401 | W-402 | S-403 | A-411 | D-415 | K-416 | R-417 | V-421 |
| CCA53915 | A-425 | W-426 | S-427 | A-435 | D-439 | K-440 | R-441 | V-445 |
| CAJ88063 | A-401 | W-402 | S-403 | A-411 | D-415 | K-416 | R-417 | V-421 |
| ABF87202 | A-403 | W-404 | S-405 | A-413 | Q-417 | K-418 | R-419 | V-423 |
| BAJ30040 | A-398 | W-399 | S-400 | A-408 | D-412 | K-413 | R-414 | I-418 |
| ACO44852 | A-392 | W-393 | S-394 | A-402 | S-406 | R-407 | R-408 | F-412 |
| AFO59750 | V-409 | W-410 | T-411 | A-419 | S-423 | K-424 | R-425 | V-429 |
| BAJ31549 | L-443 | W-444 | S-445 | A-453 | S-457 | K-458 | R-459 | V-463 |
| ACY97307 | T-431 | W-432 | S-433 | A-441 | S-445 | R-446 | R-447 | V-451 |
| ACM06095 | V-402 | W-403 | S-404 | A-412 | S-416 | K-417 | R-418 | V-422 |
| AEI64652 | A-403 | W-404 | S-405 | A-413 | Q-417 | K-418 | R-419 | V-423 |
| BAG17581 | A-415 | W-416 | S-417 | A-425 | D-429 | K-430 | R-431 | V-435 |
| AEM83530 | A-401 | W-402 | S-403 | A-411 | D-415 | K-416 | R-417 | V-421 |
| ADG89307 | C-411 | W-412 | S-413 | A-421 | A-425 | A-426 | R-427 | V-431 |
| ABG04991 | V-408 | W-409 | S-410 | A-418 | S-422 | K-423 | R-424 | V-428 |
| ABP54026 | A-428 | W-429 | S-430 | A-438 | T-442 | K-443 | R-444 | I-448 |
| ABV97405 | A-428 | W-429 | S-430 | A-438 | T-442 | K-443 | R-444 | I-448 |
| ADH60167 | V-398 | W-399 | S-400 | A-408 | S-412 | K-413 | R-414 | V-418 |
| ADV80493 | V-398 | W-399 | S-400 | A-408 | S-412 | K-413 | R-414 | V-418 |
| AEM77729 | V-398 | W-399 | S-400 | V-408 | S-412 | K-413 | R-414 | V-418 |
| ABK71329 | V-414 | W-415 | S-416 | A-424 | A-428 | K-429 | R-430 | I-434 |
| ACZ89864 | V-424 | W-425 | S-426 | A-434 | R-438 | R-439 | R-440 | V-444 |
| BAC72965 | L-428 | W-429 | S-430 | A-438 | G-442 | K-443 | R-444 | V-448 |
| ADD25173 | V-399 | W-400 | S-401 | A-409 | S-413 | K-414 | R-415 | V-419 |
| ABF44291 | A-400 | W-401 | S-402 | A-410 | E-414 | K-415 | R-416 | V-420 |
| AEY93261 | V-418 | W-419 | S-420 | A-428 | D-432 | K-433 | R-434 | V-438 |
| 1NP2 | V-384 | W-385 | S-386 | A-394 | T-398 | R-399 | R-400 | Y-404 |
| AEG34643 | V-384 | W-385 | S-386 | A-394 | T-398 | R-399 | R-400 | Y-404 |
| AFR07907 | A-415 | W-416 | S-417 | A-425 | S-429 | R-430 | R-431 | V-435 |
| AEB43702 | A-427 | W-428 | S-429 | A-437 | T-441 | K-442 | R-443 | I-447 |
| BAL92882 | A-419 | W-420 | S-421 | A-429 | A-433 | K-434 | R-435 | V-439 |
| CCA59876 | L-427 | W-428 | S-429 | A-437 | A-441 | K-442 | R-443 | V-447 |
| ACU74192 | V-421 | W-422 | S-423 | S-431 | S-435 | K-436 | R-437 | V-441 |
| ABV96319 | V-420 | W-421 | S-422 | A-430 | R-434 | K-435 | R-436 | V-440 |
| AF053528 | L-428 | W-429 | S-430 | A-438 | S-442 | K-443 | R-444 | V-448 |
| AFH40090 | V-384 | W-385 | S-386 | A-394 | T-398 | R-399 | R-400 | Y-404 |
| ADL49193 | V-408 | W-409 | S-410 | A-418 | R-422 | K-423 | R-424 | V-428 |
| AFE08200 | A-403 | W-404 | S-405 | A-413 | T-417 | K-418 | R-419 | V-423 |
| AAN05441 | V-384 | W-385 | S-386 | A-394 | T-398 | R-399 | R-400 | Y-404 |
| CAJ90043 | V-412 | W-413 | S-414 | A-422 | D-426 | K-427 | R-428 | V-432 |
| AEY89575 | L-421 | W-422 | S-423 | A-431 | G-435 | K-436 | R-437 | V-441 |
| AAF37730 | A-433 | W-434 | S-435 | A-443 | G-447 | K-448 | R-449 | V-453 |
| BAL98072 | I-405 | W-406 | S-407 | A-415 | T-419 | Q-420 | R-421 | V-425 |
| AAN05440 | V-384 | W-385 | S-386 | A-394 | T-398 | R-399 | R-400 | Y-404 |
| AEM77895 | V-398 | W-399 | S-400 | A-408 | S-412 | K-413 | R-414 | V-418 |
| ADB34272 | C-423 | W-424 | S-425 | S-433 | A-437 | R-438 | R-439 | V-443 |
| ABW87307 | V-384 | W-385 | S-386 | A-394 | T-398 | R-399 | R-400 | Y-404 |
| AF322365_1 | V-384 | W-385 | S-386 | A-394 | T-398 | R-399 | R-400 | Y-404 |
| CAC10107 | L-425 | W-426 | S-427 | A-435 | E-439 | K-440 | R-441 | V-445 |
| AEB47478 | V-410 | W-411 | S-412 | A-420 | R-424 | K-425 | R-426 | V-430 |
| ADL45220 | A-427 | W-428 | S-429 | A-437 | T-441 | K-442 | R-443 | V-447 |
| ABK51908 | L-428 | W-429 | S-430 | A-438 | S-442 | R-443 | R-444 | V-448 |
| CAN94460 | V-408 | W-409 | S-410 | D-418 | S-422 | Q-423 | R-424 | V-428 |
| AEF18219 | L-396 | W-397 | S-398 | A-406 | S-410 | K-411 | R-412 | V-416 |
| ADD39191 | L-439 | W-440 | S-441 | A-449 | A-453 | K-454 | R-455 | V-459 |
| ABP52811 | V-419 | W-420 | S-421 | A-429 | R-433 | K-434 | R-435 | V-439 |
| AEV88819 | L-434 | W-435 | S-436 | A-444 | A-448 | K-449 | R-450 | V-454 |
| AEY89570 | L-431 | W-432 | S-433 | S-441 | S-445 | K-446 | R-447 | V-451 |
| ADD01635 | V-398 | W-399 | S-400 | A-408 | S-412 | K-413 | R-414 | V-418 |
| CAB95278 | V-412 | W-413 | S-414 | A-422 | D-426 | K-427 | R-428 | V-432 |
| AEN13042 | V-412 | W-413 | S-414 | A-422 | A-426 | K-427 | R-428 | I-432 |
| ACY97750 | V-417 | W-418 | S-419 | A-427 | S-431 | K-432 | R-433 | V-437 |
| ADI10010 | L-417 | W-418 | S-419 | A-427 | S-431 | K-432 | R-433 | V-437 |
| CCB72805 | V-425 | W-426 | S-427 | S-435 | G-439 | K-440 | R-441 | V-445 |
| ACY14034 | L-419 | W-420 | S-421 | A-429 | A-433 | K-434 | R-435 | V-439 |
| AAZ55664 | V-412 | W-413 | S-414 | A-422 | E-426 | R-427 | R-428 | V-432 |
| ADW02698 | V-412 | W-413 | S-414 | A-422 | A-426 | K-427 | R-428 | I-432 |
| ABC33525 | Y-391 | W-392 | S-393 | N-401 | S-405 | K-406 | R-407 | T-411 |
| ADB34282 | A-399 | W-400 | S-401 | A-409 | A-413 | P-414 | R-415 | A-419 |
| AAM23648 | V-401 | W-402 | S-403 | A-411 | S-415 | K-416 | R-417 | V-421 |
| CCH86028 | A-416 | W-417 | S-418 | A-426 | D-430 | Q-431 | R-432 | V-436 |
| ADV67544 | A-400 | W-401 | S-402 | A-410 | K-414 | K-415 | R-416 | F-420 |
| ACZ89862 | V-412 | W-413 | S-414 | A-422 | D-426 | K-427 | R-428 | V-432 |
| ADH66252 | A-415 | W-416 | S-417 | A-425 | S-429 | R-430 | R-431 | V-435 |
| ADV80605 | V-398 | W-399 | S-400 | A-408 | S-412 | K-413 | R-414 | V-418 |
| ADW05507 | L-432 | W-433 | S-434 | A-442 | S-446 | K-447 | R-448 | V-452 |
| BAA86923 | V-384 | W-385 | S-386 | A-394 | T-398 | R-399 | R-400 | Y-404 |
| AAN05438 | V-384 | W-385 | S-386 | A-394 | T-398 | R-399 | R-400 | Y-404 |
| CAC16438 | L-429 | W-430 | S-431 | A-439 | S-443 | K-444 | R-445 | V-449 |
| CAJ89567 | L-429 | W-430 | S-431 | A-439 | S-443 | K-444 | R-445 | V-449 |
| AFD27167 | A-408 | W-409 | S-410 | A-418 | E-422 | K-423 | R-424 | V-428 |
| BAG20044 | L-426 | W-427 | S-428 | A-436 | S-440 | K-441 | R-442 | V-446 |
| ACU71435 | A-410 | W-411 | S-412 | A-420 | D-424 | K-425 | R-426 | I-430 |
| ADG87563 | V-437 | W-438 | S-439 | A-447 | H-451 | K-452 | R-453 | V-457 |
| ABI35984 | V-384 | W-385 | S-386 | A-394 | T-398 | R-399 | R-400 | Y-404 |
| CAB42553 | V-384 | W-385 | S-386 | A-394 | T-398 | R-399 | R-400 | Y-404 |
| 1UG6 | V-384 | W-385 | S-386 | A-394 | T-398 | R-399 | R-400 | Y-404 |
| CAA91220 | V-401 | W-402 | S-403 | A-411 | S-415 | K-416 | R-417 | V-421 |
| AFK85369 | L-397 | W-398 | S-399 | A-407 | S-411 | K-412 | R-413 | V-417 |
| AEN10177 | L-433 | W-434 | S-435 | A-443 | S-447 | K-448 | R-449 | V-453 |
| AEM87460 | L-418 | W-419 | S-420 | A-428 | S-432 | K-433 | R-434 | V-438 |
| AAZ81839 | V-399 | W-400 | S-401 | A-409 | T-413 | K-414 | R-415 | V-419 |
| ADU50085 | Y-443 | W-444 | S-445 | A-453 | A-457 | K-458 | R-459 | V-463 |
| AEK47062 | A-419 | W-420 | S-421 | A-429 | A-433 | K-434 | R-435 | I-439 |
| ABD68852 | A-410 | W-411 | S-412 | A-420 | I-424 | R-425 | R-426 | T-430 |
| ACL38401 | A-429 | W-430 | S-431 | S-439 | H-443 | Q-444 | R-445 | V-449 |
| ABS05424 | L-434 | W-435 | S-436 | G-444 | S-448 | K-449 | R-450 | V-454 |
| AEK43773 | Y-406 | W-407 | S-408 | A-416 | A-420 | K-421 | R-422 | V-426 |
| AEV87561 | A-392 | W-393 | S-394 | A-402 | Q-406 | R-407 | R-408 | V-412 |
| ACZ86244 | A-422 | W-423 | S-424 | A-432 | G-436 | K-437 | R-438 | V-442 |
| AEB46173 | V-417 | W-418 | S-419 | A-427 | D-431 | R-432 | R-433 | I-437 |
| BAG21567 | L-434 | W-435 | S-436 | G-444 | A-448 | K-449 | R-450 | V-454 |
| ABX05041 | V-398 | W-399 | S-400 | A-408 | S-412 | Q-413 | R-414 | I-418 |
| AFR09943 | V-429 | W-430 | S-431 | A-439 | E-443 | R-444 | R-445 | V-449 |
| ACZ89285 | C-412 | W-413 | S-414 | A-422 | D-426 | A-427 | R-428 | V-432 |
| ACV58907 | V-396 | W-397 | S-398 | A-406 | T-410 | K-411 | R-412 | V-416 |
| ADG89462 | V-378 | W-379 | S-380 | A-388 | H-392 | Q-393 | R-394 | V-398 |
| ADU09756 | Y-426 | W-427 | S-428 | A-436 | G-440 | P-441 | R-442 | A-446 |
| ACZ90607 | V-388 | W-389 | S-390 | A-398 | H-402 | Q-403 | R-404 | V-408 |
| ADG73989 | V-426 | W-427 | S-428 | A-436 | D-440 | R-441 | R-442 | V-446 |
| AEG45154 | A-465 | W-466 | S-467 | A-475 | S-479 | K-480 | R-481 | V-485 |
| AEJ43907 | V-396 | W-397 | S-398 | A-406 | T-410 | K-411 | R-412 | V-416 |
| ACZ20790 | A-443 | W-444 | S-445 | A-453 | N-457 | K-458 | R-459 | V-463 |
| CCB77455 | V-399 | W-400 | T-401 | A-409 | H-413 | Q-414 | R-415 | V-419 |
| ADL46625 | A-421 | W-422 | S-423 | A-431 | D-435 | K-436 | R-437 | V-441 |
| ADU10772 | A-421 | W-422 | S-423 | A-431 | D-435 | K-436 | R-437 | V-441 |
| ADD45899 | V-418 | W-419 | S-420 | A-428 | T-432 | K-433 | R-434 | V-438 |
| CCA53920 | A-421 | W-422 | S-423 | A-431 | D-435 | K-436 | R-437 | V-441 |
| BAJ28512 | L-435 | W-436 | S-437 | A-445 | S-449 | K-450 | R-451 | V-455 |
| ACL70277 | V-400 | W-401 | S-402 | A-410 | S-414 | K-415 | R-416 | I-420 |
| CBG72797 | L-425 | W-426 | S-427 | A-435 | G-439 | K-440 | R-441 | V-445 |
| AEW05616 | V-402 | W-403 | S-404 | A-412 | S-416 | K-417 | R-418 | I-422 |
| AEB46623 | V-393 | W-394 | S-395 | A-403 | T-407 | K-408 | R-409 | V-413 |
| 1GNX | L-429 | W-430 | S-431 | A-439 | S-443 | K-444 | R-445 | V-449 |
| ACV76621 | L-421 | W-422 | S-423 | A-431 | D-435 | R-436 | R-437 | I-441 |
| ACZ31628 | A-464 | W-465 | S-466 | A-474 | T-478 | K-479 | R-480 | V-484 |
| ADH67953 | V-409 | W-410 | S-411 | A-419 | D-423 | R-424 | R-425 | V-429 |
| ADB34290 | A-420 | W-421 | S-422 | A-430 | E-434 | K-435 | R-436 | V-440 |
| ADU09106 | V-422 | W-423 | S-424 | A-432 | T-436 | K-437 | R-438 | V-442 |
| CAA82733 | L-429 | W-430 | S-431 | A-439 | S-443 | K-444 | R-445 | V-449 |
| CBG67455 | L-446 | W-447 | S-448 | G-456 | S-460 | K-461 | R-462 | V-466 |
| CAN00920 | V-438 | W-439 | S-440 | A-448 | S-452 | K-453 | R-454 | V-458 |
| ADX71280 | A-427 | W-428 | S-429 | S-437 | H-441 | Q-442 | R-443 | V-447 |
| ACQ81085 | L-447 | W-448 | S-449 | A-457 | S-461 | K-462 | R-463 | V-467 |
| 3AHX | I-398 | W-399 | S-400 | A-408 | E-412 | K-413 | R-414 | V-418 |
| ACU35632 | A-419 | W-420 | S-421 | S-429 | E-433 | K-434 | R-435 | V-439 |
| AEV86556 | Y-408 | W-409 | S-410 | A-418 | G-422 | P-423 | R-424 | V-428 |
| AEG45006 | A-433 | W-434 | S-435 | S-443 | S-447 | K-448 | R-449 | L-453 |
| BAG18801 | A-420 | W-421 | S-422 | A-430 | D-434 | K-435 | R-436 | V-440 |
| ADI03707 | C-420 | W-421 | S-422 | A-430 | G-434 | Q-435 | R-436 | V-440 |
| CAQ00266 | V-439 | W-440 | S-441 | A-449 | S-453 | K-454 | R-455 | V-459 |
| ADD43929 | T-398 | W-399 | T-400 | A-408 | H-412 | Q-413 | R-414 | V-418 |
| ADJ49823 | A-405 | W-406 | S-407 | A-415 | T-419 | Q-420 | R-421 | V-425 |
| ABX05062 | A-404 | W-405 | S-406 | A-414 | D-418 | E-419 | R-420 | I-424 |
| ADL48215 | V-398 | W-399 | S-400 | A-408 | T-412 | K-413 | R-414 | V-418 |
| ACU70272 | T-403 | W-404 | T-405 | A-413 | H-417 | Q-418 | R-419 | V-423 |
| ADG88606 | V-424 | W-425 | S-426 | A-434 | G-438 | K-439 | R-440 | I-444 |
| AEW47954 | V-399 | W-400 | S-401 | G-409 | T-413 | Q-414 | F-415 | V-419 |
| ACD20223 | L-420 | W-421 | S-422 | A-430 | E-434 | R-435 | R-436 | V-440 |
| AFC28171 | C-398 | W-399 | S-400 | A-408 | S-412 | K-413 | R-414 | V-418 |
| ADW03239 | A-419 | W-420 | S-421 | A-429 | D-433 | K-434 | R-435 | V-439 |
| CBT74727 | V-423 | W-424 | S-425 | A-433 | A-437 | K-438 | R-439 | I-443 |
| CAM04686 | A-412 | W-413 | S-414 | A-422 | T-426 | Q-427 | R-428 | V-432 |
| ACZ20966 | A-424 | W-425 | S-426 | G-434 | D-438 | R-439 | R-440 | V-444 |
| AEV38153 | C-414 | W-415 | T-416 | A-424 | L-428 | P-429 | R-430 | I-434 |
| BAJ26623 | T-394 | W-395 | S-396 | G-404 | G-408 | E-409 | R-410 | V-414 |
| BAL91665 | A-392 | W-393 | S-394 | A-402 | T-406 | K-407 | R-408 | V-412 |
| ACV09397 | A-422 | W-423 | S-424 | G-432 | D-436 | R-437 | R-438 | I-442 |
| ADG20157 | A-393 | W-394 | S-395 | A-403 | D-407 | K-408 | R-409 | V-413 |
| ADC61565 | V-396 | W-397 | S-398 | A-406 | S-410 | K-411 | R-412 | Y-416 |
| ACM66669 | V-423 | W-424 | S-425 | A-433 | A-437 | K-438 | R-439 | I-443 |
| ABD68843 | V-398 | W-399 | S-400 | A-408 | A-412 | K-413 | R-414 | V-418 |
| ABS61373 | I-394 | W-395 | S-396 | A-404 | S-408 | K-409 | R-410 | V-414 |
| CBA30283 | Y-403 | W-404 | S-405 | A-413 | D-417 | K-418 | R-419 | V-423 |
| AAA22266 | E-401 | W-402 | S-403 | A-411 | G-415 | M-416 | R-417 | V-421 |
| CAA42814 | L-401 | W-402 | S-403 | A-411 | N-415 | K-416 | R-417 | V-421 |
| ABN51453 | L-424 | W-425 | S-426 | A-434 | N-438 | K-439 | R-440 | V-444 |
| ACZ00292 | V-398 | W-399 | S-400 | S-408 | G-412 | P-413 | R-414 | V-418 |
| AEI12946 | V-426 | W-427 | S-428 | S-436 | D-440 | R-441 | R-442 | V-446 |
| ABS15474 | A-414 | W-415 | T-416 | A-424 | T-428 | T-429 | P-430 | T-434 |
| ADL51094 | I-398 | W-399 | S-400 | A-408 | E-412 | K-413 | R-414 | V-418 |
| AFG34202 | I-394 | W-395 | S-396 | A-404 | S-408 | K-409 | R-410 | I-414 |
| AAN60220 | I-394 | W-395 | S-396 | A-404 | S-408 | K-409 | R-410 | I-414 |

| Corresp. | Sequence name | | | | | | |
|---|---|---|---|---|---|---|---|
| Sequence Pos. | L427 | E428 | T441 | L447 | A449 | E450 | K451 |
| SEQ ID NO. 379/380 | L-427 | E-428 | T-441 | L-447 | A-449 | E-450 | K-451 |
| SEQ ID NO. 378 | Q-425 | E-426 | F-439 | — | A-444 | E-445 | A-446 |
| US8101393-0094 | L-427 | E-428 | T-441 | L-447 | V-449 | D-450 | N-451 |
| US8101393-0388 | L-427 | E-428 | T-441 | L-447 | A-449 | E-450 | K-451 |
| US8101393-0172 | Q-424 | E-425 | F-438 | E-444 | G-446 | — | — |
| JP2011205992-0018 | L-425 | E-426 | F-439 | G-445 | — | — | — |
| JP2011205992-0023 | Q-422 | R-423 | F-436 | A-442 | S-444 | — | — |
| JP2011205992-0022 | Q-427 | E-428 | I-441 | — | — | — | — |
| US20110214199-58656 | Q-426 | V-427 | I-440 | G-446 | A-448 | R-449 | R-450 |
| US20110214199-62406 | Q-442 | V-443 | I-456 | G-462 | E-464 | R-465 | K-466 |
| US20110214199-47919 | Q-430 | R-431 | L-444 | — | — | L-450 | D-451 |
| US20110214199-60662 | Q-435 | R-436 | V-449 | P-455 | G-457 | — | — |
| US20110214199-36660 | Q-441 | V-442 | V-455 | V-461 | H-463 | V-464 | F-465 |
| US20110214199-17908 | L-455 | A-456 | A-469 | L-475 | G-477 | A-478 | T-479 |
| US20110214199-25308 | Q-411 | R-412 | R-425 | T-431 | G-433 | S-434 | A-435 |
| US20110214199-64004 | Q-411 | R-412 | R-425 | T-431 | G-433 | S-434 | A-435 |
| US20110214199-25023 | Q-460 | T-461 | V-474 | I-480 | G-482 | Q-483 | E-484 |
| US20110214199-52644 | Q-411 | R-412 | R-425 | L-431 | — | — | — |
| US20110214199-13862 | Q-452 | Q-453 | A-466 | L-472 | P-474 | V-475 | D-476 |
| US20110214199-29446 | Q-428 | K-429 | V-442 | I-448 | — | — | — |
| US20110214199-5988 | Q-411 | R-412 | R-425 | T-431 | — | — | — |
| US20110214199-23246 | Q-411 | R-412 | R-425 | T-431 | — | — | — |
| US20110214199-12000 | Q-425 | E-426 | L-439 | K-445 | — | — | — |
| US20110214199-56949 | Q-421 | K-422 | F-435 | — | — | — | — |
| US8101393-0272 | Q-444 | R-445 | V-458 | L-464 | G-466 | A-467 | — |
| US8101393-0014 | Q-435 | Q-436 | A-449 | — | — | F-455 | A-456 |
| US8101393-0398 | Q-454 | K-455 | V-468 | L-474 | A-476 | Q-477 | — |
| US8101393-0492 | Q-454 | K-455 | V-468 | L-474 | A-476 | Q-477 | — |
| US8101393-0400 | Q-454 | A-455 | V-468 | L-474 | A-476 | Q-477 | — |
| US8101393-0266 | L-454 | E-455 | V-468 | L-474 | A-476 | P-477 | — |
| US8101393-0366 | G-409 | R-410 | — | — | — | — | — |
| US8101393-0342 | Q-434 | R-435 | V-448 | — | — | V-454 | Q-455 |
| US8101393-0356 | Q-424 | K-425 | V-438 | — | L-444 | I-445 | D-446 |
| US8101393-0320 | L-428 | E-429 | V-442 | F-448 | — | — | — |
| US7314974-19451 | Q-428 | R-429 | L-442 | — | — | L-448 | D-449 |
| US7314974-8251 | Q-435 | T-436 | — | — | — | — | — |
| US7630836-9340 | Q-426 | V-427 | I-440 | G-446 | A-448 | R-449 | R-450 |
| US7630836-12787 | L-455 | A-456 | A-469 | L-475 | G-477 | A-478 | T-479 |
| US20090220480-0018 | Q-430 | A-431 | I-444 | L-450 | E-452 | L-453 | E-454 |
| US20120034253-0080 | Q-411 | R-412 | R-425 | L-431 | — | — | — |
| US20120034253-0077 | Q-411 | R-412 | R-425 | T-431 | — | — | — |
| US20120034253-0078 | Q-411 | R-412 | R-425 | T-431 | — | — | — |
| WO2012016960-11369 | Q-461 | Q-462 | V-475 | L-481 | — | — | — |
| US20120015408-0002 | Q-428 | K-429 | V-442 | I-448 | D-450 | — | — |
| US20120015408-0004 | Q-428 | K-429 | V-442 | I-448 | D-450 | — | — |
| US20120015408-0006 | Q-428 | K-429 | V-442 | I-448 | D-450 | — | — |
| US20120015408-0008 | Q-428 | K-429 | V-442 | I-448 | D-450 | — | — |
| US20120015408-0010 | Q-428 | K-429 | V-442 | I-448 | D-450 | — | — |
| US20120015408-0012 | Q-428 | K-429 | V-442 | I-448 | D-450 | — | — |
| US20120015408-0014 | Q-428 | K-429 | V-442 | I-448 | D-450 | — | — |
| US20120015408-0016 | Q-428 | K-429 | V-442 | I-448 | D-450 | — | — |
| US20120015408-0018 | Q-428 | K-429 | V-442 | I-448 | D-450 | — | — |
| US20120015408-0020 | Q-428 | K-429 | V-442 | I-448 | D-450 | — | — |
| US20120015408-0022 | Q-428 | K-429 | V-442 | I-448 | D-450 | — | — |
| US20120015408-0026 | Q-428 | K-429 | V-442 | I-448 | D-450 | — | — |
| US20120015408-0028 | Q-428 | K-429 | V-442 | I-448 | D-450 | — | — |
| US20120015408-0030 | Q-428 | K-429 | V-442 | I-448 | D-450 | Q-451 | — |
| US20120015408-0032 | Q-428 | K-429 | V-442 | I-448 | D-450 | Q-451 | — |
| US20120015408-0034 | Q-428 | K-429 | V-442 | I-448 | D-450 | Q-451 | — |
| US20120015408-0036 | Q-428 | K-429 | V-442 | I-448 | D-450 | — | — |
| US20120015408-0038 | Q-428 | K-429 | V-442 | I-448 | D-450 | Q-451 | — |
| US20120015408-0040 | Q-428 | K-429 | V-442 | I-448 | D-450 | — | — |
| US20120015408-0042 | Q-428 | K-429 | V-442 | I-448 | D-450 | Q-451 | — |
| US20120015408-0044 | Q-428 | K-429 | V-442 | I-448 | D-450 | — | — |
| US20120015408-0046 | Q-428 | K-429 | V-442 | I-448 | D-450 | Q-451 | — |
| US20120015408-0048 | Q-428 | K-429 | V-442 | I-448 | D-450 | Q-451 | — |
| US20120015408-0050 | Q-428 | K-429 | V-442 | I-448 | D-450 | Q-451 | — |
| US20120015408-0052 | Q-428 | N-429 | V-442 | I-448 | D-450 | Q-451 | — |
| US20120015408-0054 | Q-428 | K-429 | V-442 | I-448 | D-450 | — | — |
| US20120015408-0056 | Q-428 | K-429 | V-442 | I-448 | D-450 | Q-451 | — |
| US20120015408-0058 | Q-428 | K-429 | V-442 | I-448 | D-450 | — | — |
| US20120015408-0060 | Q-428 | K-429 | V-442 | I-448 | D-450 | Q-451 | — |
| US20120015408-0062 | Q-428 | K-429 | V-442 | I-448 | D-450 | Q-451 | — |
| US20120015408-0064 | Q-428 | K-429 | V-442 | I-448 | D-450 | — | — |
| US20120015408-0066 | Q-428 | K-429 | V-442 | I-448 | D-450 | — | — |
| US20120015408-0068 | Q-428 | K-429 | V-442 | I-448 | D-450 | Q-451 | — |
| US20120015408-0070 | Q-428 | K-429 | V-442 | I-448 | D-450 | Q-451 | — |
| US20120015408-0072 | Q-428 | K-429 | V-442 | I-448 | D-450 | Q-451 | — |
| US20120015408-0074 | Q-428 | K-429 | V-442 | I-448 | D-450 | Q-451 | — |
| US20120015408-0076 | Q-428 | K-429 | V-442 | I-448 | D-450 | Q-451 | — |
| US20120015408-0078 | Q-428 | K-429 | V-442 | I-448 | D-450 | Q-451 | — |
| US20120015408-0080 | Q-428 | K-429 | V-442 | I-448 | D-450 | — | — |
| US20120015408-0082 | Q-428 | K-429 | V-442 | I-448 | D-450 | Q-451 | — |
| US20120015408-0086 | Q-428 | K-429 | V-442 | I-448 | D-450 | — | — |
| US20120015408-0084 | Q-428 | K-429 | V-442 | I-448 | D-450 | — | — |
| US20120015408-0088 | Q-428 | K-429 | V-442 | I-448 | D-450 | Q-451 | — |
| US20120015408-0090 | Q-428 | K-429 | V-442 | I-448 | D-450 | Q-451 | — |
| US20120015408-0092 | Q-428 | K-429 | V-442 | I-448 | D-450 | Q-451 | — |
| US20120015408-0094 | Q-428 | K-429 | V-442 | I-448 | D-450 | — | — |
| US20120015408-0096 | Q-428 | K-429 | V-442 | I-448 | D-450 | — | — |
| US20120015408-0098 | Q-428 | K-429 | V-442 | I-448 | D-450 | Q-451 | — |
| US20120015408-0100 | Q-428 | K-429 | V-442 | I-448 | D-450 | Q-451 | — |
| US20120015408-0102 | Q-428 | K-429 | V-442 | I-448 | D-450 | Q-451 | — |
| US20120015408-0104 | Q-428 | K-429 | V-442 | I-448 | D-450 | Q-451 | — |
| US20120015408-0106 | Q-428 | K-429 | V-442 | I-448 | D-450 | Q-451 | — |
| US20120015408-0108 | Q-428 | K-429 | V-442 | I-448 | D-450 | Q-451 | — |
| US20120015408-0110 | Q-428 | K-429 | V-442 | I-448 | D-450 | Q-451 | — |
| US20120015408-0112 | Q-428 | K-429 | V-442 | I-448 | D-450 | Q-451 | — |
| US20120015408-0114 | Q-428 | K-429 | V-442 | I-448 | D-450 | Q-451 | — |
| US20120015408-0116 | Q-428 | K-429 | V-442 | I-448 | D-450 | Q-451 | — |
| US20120015408-0118 | Q-428 | K-429 | A-442 | I-448 | D-450 | P-451 | — |
| US20120015408-0120 | Q-428 | K-429 | V-442 | I-448 | D-450 | Q-451 | — |
| US20120015408-0122 | Q-428 | K-429 | V-442 | I-448 | D-450 | Q-451 | — |
| US20120015408-0124 | Q-428 | K-429 | V-442 | I-448 | D-450 | — | — |
| US8202716-0464 | L-426 | A-427 | V-440 | L-446 | P-448 | A-449 | E-450 |
| US20110262988-0002 | L-428 | E-429 | V-442 | F-448 | E-450 | H-451 | H-452 |
| US20110262988-0003 | L-451 | E-452 | V-465 | F-471 | — | — | — |
| US20110151538-0130 | L-480 | E-481 | V-494 | F-500 | E-502 | H-503 | H-504 |
| US20100003234-0048 | Q-428 | K-429 | V-442 | L-448 | L-450 | — | — |
| US6184018-0012 | L-428 | E-429 | V-442 | F-448 | — | — | — |
| US6377893-0063 | L-423 | E-424 | V-437 | — | — | — | — |
| ACJ34717 | L-438 | E-439 | T-452 | L-458 | D-460 | Q-461 | — |
| ADD27066 | Q-422 | R-423 | F-436 | A-442 | S-444 | — | — |
| ACZ42845 | L-431 | E-432 | V-445 | L-451 | P-453 | T-454 | E-455 |
| ABQ91969 | Q-425 | R-426 | V-439 | V-445 | E-447 | G-448 | — |
| ABTJ56651 | Q-425 | R-426 | V-439 | L-445 | E-447 | T-448 | Q-449 |
| AEY92801 | Q-428 | V-429 | I-442 | G-448 | T-450 | R-451 | R-452 |
| CAD55382 | Q-442 | V-443 | I-456 | G-462 | E-464 | R-465 | K-466 |
| AD073143 | Q-434 | Q-435 | V-448 | — | — | L-454 | S-455 |
| CCA60311 | Q-444 | R-445 | V-458 | L-464 | G-466 | A-467 | — |
| ADI15206 | Q-432 | E-433 | V-446 | Q-452 | L-454 | E-455 | V-456 |
| ADI12494 | Q-428 | A-429 | I-442 | D-448 | A-450 | R-451 | H-452 |
| ACU35736 | Q-443 | V-444 | V-457 | L-463 | A-465 | P-466 | S-467 |
| BAC69512 | Q-426 | V-427 | I-440 | G-446 | A-448 | R-449 | R-450 |
| AEN08263 | Q-428 | K-429 | I-442 | — | A-445 | H-446 | R-447 |
| CCA53915 | Q-452 | R-453 | H-466 | G-472 | A-474 | H-475 | R-476 |
| CAJ88063 | Q-428 | A-429 | I-442 | — | A-445 | H-446 | R-447 |
| ABF87202 | Q-430 | R-431 | L-444 | — | — | L-450 | D-451 |
| BAJ30040 | Q-425 | V-426 | L-439 | A-445 | A-447 | — | — |
| ACO44852 | Q-419 | E-420 | L-433 | V-439 | A-441 | D-442 | — |
| AFO59750 | Q-436 | V-437 | I-450 | L-456 | — | — | — |
| BAJ31549 | Q-470 | R-471 | V-484 | L-490 | Q-492 | — | — |
| ACY97307 | Q-458 | R-459 | V-472 | L-478 | D-480 | R-481 | H-482 |
| ACM06095 | L-429 | S-430 | I-443 | L-449 | E-451 | G-452 | — |
| AEI64652 | Q-430 | R-431 | L-444 | — | — | L-450 | D-451 |
| BAG17581 | Q-442 | R-443 | L-456 | E-462 | R-464 | G-465 | G-466 |
| AEM83530 | Q-428 | T-429 | I-442 | G-448 | A-450 | R-451 | R-452 |
| ADG89307 | Q-438 | A-439 | F-452 | P-458 | G-460 | P-461 | D-462 |
| ABG04991 | Q-435 | R-436 | V-449 | P-455 | G-457 | — | — |
| ABP54026 | Q-455 | A-456 | T-469 | L-475 | A-477 | Q-478 | — |
| ABV97405 | Q-455 | V-456 | V-469 | L-475 | A-477 | Q-478 | — |
| ADH60167 | Q-425 | K-426 | V-439 | I-445 | E-447 | — | — |
| ADV80493 | Q-425 | K-426 | V-439 | I-445 | — | — | — |
| AEM77729 | Q-425 | K-426 | V-439 | I-445 | Y-447 | — | — |
| ABK71329 | Q-441 | V-442 | V-455 | V-461 | H-463 | V-464 | F-465 |
| ACZ89864 | Q-451 | R-452 | V-465 | L-471 | G-473 | N-474 | — |
| BAC72965 | L-455 | A-456 | A-469 | L-475 | G-477 | A-478 | T-479 |
| ADD25173 | Q-426 | K-427 | V-440 | M-446 | — | — | — |
| ABF44291 | Q-427 | T-428 | F-441 | — | — | — | — |
| AEY93261 | Q-445 | R-446 | M-459 | G-465 | — | — | — |
| 1NP2 | Q-411 | R-412 | R-425 | T-431 | G-433 | S-434 | A-435 |
| AEG34643 | Q-411 | R-412 | R-425 | T-431 | G-433 | S-434 | A-435 |
| AFR07907 | Q-442 | E-443 | L-456 | F-462 | E-464 | R-465 | — |
| AEB43702 | Q-454 | T-455 | V-468 | L-474 | A-476 | Q-477 | — |
| BAL92882 | Q-446 | Q-447 | V-460 | L-466 | — | — | — |
| CCA59876 | L-454 | E-455 | V-468 | L-474 | A-476 | P-477 | — |
| ACU74192 | G-448 | K-449 | I-462 | S-468 | L-470 | — | — |
| ABV96319 | Q-447 | R-448 | V-461 | L-467 | — | — | — |
| AF053528 | G-455 | T-456 | V-469 | L-475 | G-477 | A-478 | — |
| AFH40090 | Q-411 | R-412 | R-425 | T-431 | G-433 | S-434 | A-435 |
| ADL49193 | Q-435 | R-436 | V-449 | L-455 | — | — | — |
| AFE08200 | Q-430 | Q-431 | V-444 | — | — | L-450 | D-451 |
| AAN05441 | Q-411 | R-412 | R-425 | T-431 | G-433 | S-434 | A-435 |
| CAJ90043 | Q-439 | E-440 | M-453 | G-459 | — | — | — |
| AEY89575 | L-448 | E-449 | A-462 | L-468 | P-470 | V-471 | A-472 |
| AAF37730 | Q-460 | T-461 | V-474 | I-480 | G-482 | Q-483 | E-484 |
| BAL98072 | Q-432 | A-433 | V-446 | — | — | V-452 | E-453 |
| AAN05440 | Q-411 | R-412 | R-425 | L-431 | — | — | — |
| AEM77895 | Q-425 | K-426 | V-439 | I-445 | E-447 | — | — |
| ADB34272 | Q-450 | T-451 | L-464 | L-469 | T-471 | — | — |
| ABW87307 | Q-411 | R-412 | R-425 | T-431 | — | — | — |
| AF322365_1 | Q-411 | R-412 | R-425 | L-431 | — | — | — |
| CAC10107 | Q-452 | Q-453 | A-466 | L-472 | P-474 | V-475 | D-476 |
| AEB47478 | Q-437 | R-438 | V-451 | L-457 | — | — | — |
| ADL45220 | Q-454 | A-455 | V-468 | L-474 | A-476 | Q-477 | — |
| ABK51908 | Q-455 | E-456 | V-469 | I-475 | A-477 | P-478 | — |
| CAN94460 | Q-435 | R-436 | V-449 | — | — | V-455 | D-456 |
| AEF18219 | Q-423 | K-424 | V-437 | I-443 | N-445 | I-446 | — |
| ADD39191 | Q-466 | A-467 | L-480 | L-486 | R-488 | E-489 | — |
| ABP52811 | Q-446 | R-447 | V-460 | L-466 | — | — | — |
| AEV88819 | Q-461 | Q-462 | V-475 | L-481 | — | — | — |
| AEY89570 | Q-458 | R-459 | V-472 | L-478 | A-480 | — | — |
| ADD01635 | Q-425 | K-426 | V-439 | I-445 | E-447 | — | — |
| CAB95278 | Q-439 | R-440 | M-453 | G-459 | — | — | — |
| AEN13042 | L-439 | E-440 | L-453 | A-459 | — | — | — |
| ACY97750 | Q-444 | R-445 | V-458 | R-464 | H-466 | Q-467 | A-468 |
| ADI10010 | Q-444 | T-445 | V-458 | V-464 | D-466 | A-467 | — |
| CCB72805 | Q-452 | V-453 | V-466 | L-472 | P-474 | A-475 | G-476 |
| ACY14034 | Q-446 | K-447 | A-460 | A-466 | — | — | — |
| AAZ55664 | L-439 | E-440 | L-453 | A-459 | H-461 | R-462 | G-463 |
| ADW02698 | L-439 | E-440 | L-453 | A-459 | — | — | — |
| ABC33525 | Q-418 | Q-419 | F-432 | A-438 | — | — | — |
| ADB34282 | G-426 | T-427 | L-440 | E-446 | — | — | — |
| AAM23648 | Q-428 | K-429 | V-442 | I-448 | — | — | — |
| CCH86028 | Q-443 | V-444 | V-457 | D-463 | V-465 | — | — |
| ADV67544 | Q-427 | M-428 | F-441 | A-447 | H-449 | A-450 | P-451 |
| ACZ89862 | Q-439 | N-440 | V-453 | L-459 | — | — | — |
| ADH66252 | Q-442 | R-443 | L-456 | F-462 | E-464 | R-465 | Q-466 |
| ADV80605 | Q-425 | K-426 | V-439 | I-445 | D-447 | — | — |
| ADW05507 | Q-459 | R-460 | V-473 | L-479 | P-481 | — | V-482 |
| BAA86923 | Q-411 | R-412 | — | — | — | — | — |
| AAN05438 | Q-411 | R-412 | R-425 | T-431 | — | — | — |
| CAC16438 | G-456 | T-457 | V-470 | L-476 | G-478 | A-479 | — |
| CAJ89567 | G-456 | T-457 | V-470 | L-476 | G-478 | A-479 | — |
| AFD27167 | Q-435 | E-436 | F-449 | — | — | — | — |
| BAG20044 | Q-453 | R-454 | A-467 | L-473 | R-475 | — | — |
| ACU71435 | Q-437 | T-438 | T-451 | R-457 | R-459 | G-460 | D-461 |
| ADG87563 | M-464 | R-465 | V-478 | L-484 | N-486 | G-487 | E-488 |
| ABI35984 | Q-411 | R-412 | R-425 | T-431 | — | — | — |
| CAB42553 | Q-411 | R-412 | R-425 | T-431 | — | — | — |
| 1UG6 | Q-411 | R-412 | R-425 | T-431 | — | — | — |
| CAA91220 | Q-428 | K-429 | V-442 | I-448 | D-450 | — | — |
| AFK85369 | Q-424 | K-425 | V-438 | I-444 | F-446 | — | — |
| AEN10177 | Q-460 | R-461 | V-474 | L-480 | P-482 | G-483 | D-484 |
| AEM87460 | Q-445 | A-446 | V-459 | L-465 | A-467 | A-468 | — |
| AAZ81839 | L-426 | A-427 | V-440 | L-446 | P-448 | A-449 | E-450 |
| ADU50085 | Q-470 | E-471 | I-484 | L-490 | V-492 | V-493 | S-494 |
| AEK47062 | Q-446 | Q-447 | T-460 | G-466 | — | — | — |
| ABD68852 | Q-437 | Q-438 | F-451 | — | — | — | — |
| ACL38401 | Q-456 | D-457 | V-470 | L-476 | A-478 | G-479 | S-480 |
| ABS05424 | Q-461 | E-462 | V-475 | L-481 | A-483 | A-484 | D-485 |
| AEK43773 | Q-433 | R-434 | V-447 | L-453 | — | — | — |
| AEV87561 | Q-419 | R-420 | R-433 | — | — | — | — |
| ACZ86244 | Q-449 | L-450 | T-463 | L-469 | G-471 | P-472 | A-473 |
| AEB46173 | Q-444 | Q-445 | L-458 | L-464 | — | — | — |
| BAG21567 | Q-461 | L-462 | V-475 | L-481 | P-483 | E-484 | A-485 |
| ABX05041 | Q-425 | Q-426 | V-439 | — | — | V-445 | Q-446 |
| AFR09943 | L-456 | T-457 | L-470 | R-476 | R-478 | A-479 | T-480 |
| ACZ89285 | Q-439 | A-440 | F-453 | — | — | — | — |
| ACV58907 | L-423 | A-424 | V-437 | L-443 | P-445 | A-446 | E-447 |
| ADG89462 | G-405 | A-406 | L-419 | G-425 | G-427 | P-428 | E-429 |
| ADU09756 | G-453 | S-454 | R-467 | K-473 | T-475 | S-476 | T-477 |
| ACZ90607 | G-415 | E-416 | R-429 | G-435 | R-437 | — | — |
| ADG73989 | Q-453 | V-454 | L-467 | I-473 | T-475 | P-476 | E-477 |
| AEG45154 | Q-492 | E-493 | V-506 | L-512 | A-514 | R-515 | T-516 |
| AEJ43907 | L-423 | A-424 | V-437 | L-443 | P-445 | M-446 | E-447 |
| ACZ20790 | Q-470 | E-471 | V-484 | V-490 | P-492 | R-493 | G-494 |
| CCB77455 | Q-426 | E-427 | L-440 | G-446 | R-448 | T-449 | — |
| ADL46625 | Q-448 | R-449 | T-462 | G-468 | R-470 | — | — |
| ADU10772 | Q-448 | R-449 | T-462 | G-468 | R-470 | — | — |
| ADD45899 | Q-445 | R-446 | L-459 | — | — | — | — |
| CCA53920 | Q-448 | T-449 | T-462 | A-468 | G-470 | G-471 | H-472 |
| BAJ28512 | Q-462 | R-463 | V-476 | L-482 | — | — | — |
| ACL70277 | N-428 | R-429 | V-442 | V-448 | A-450 | N-451 | — |
| CBG72797 | L-452 | T-453 | A-466 | L-472 | P-474 | L-475 | A-476 |
| AEW05616 | Q-429 | T-430 | M-443 | — | — | — | — |
| AEB46623 | G-420 | R-421 | V-434 | R-440 | T-442 | A-443 | R-444 |
| 1GNX | G-456 | T-457 | V-470 | L-476 | T-478 | A-479 | — |
| ACV76621 | Q-448 | E-449 | L-462 | L-468 | P-470 | V-471 | D-472 |
| ACZ31628 | Q-491 | E-492 | V-505 | V-511 | A-513 | R-514 | R-515 |
| ADH67953 | L-436 | E-437 | F-450 | A-456 | R-458 | V-459 | Q-460 |
| ADB34290 | Q-447 | A-448 | L-461 | R-467 | — | — | — |
| ADU09106 | Q-449 | R-450 | L-463 | — | — | — | — |
| CAA82733 | G-456 | T-457 | V-470 | L-476 | T-478 | A-479 | — |
| CBG67455 | G-473 | T-474 | V-487 | L-493 | G-495 | A-496 | — |
| CAN00920 | Q-465 | E-466 | L-479 | I-485 | P-487 | V-488 | D-489 |
| ADX71280 | Q-454 | E-455 | V-468 | V-474 | D-476 | A-477 | D-478 |
| ACQ81085 | L-474 | R-475 | V-488 | L-494 | V-496 | D-497 | G-498 |
| 3AHX | Q-425 | E-426 | L-439 | K-445 | E-447 | H-448 | H-449 |
| ACU35632 | Q-446 | V-447 | V-460 | E-466 | — | — | — |
| AEV86556 | G-435 | E-436 | R-449 | G-455 | — | — | — |
| AEG45006 | G-460 | E-461 | L-474 | L-480 | G-482 | L-483 | T-484 |
| BAG18801 | Q-447 | V-448 | T-461 | R-467 | R-469 | A-470 | — |
| ADI03707 | Q-447 | E-448 | L-461 | A-467 | T-469 | Q-470 | E-471 |
| CAQ00266 | Q-466 | E-467 | L-480 | I-486 | A-488 | V-489 | D-490 |
| ADD43929 | Q-425 | K-426 | M-439 | A-445 | — | — | — |
| ADJ49823 | Q-432 | R-433 | V-446 | L-452 | T-454 | P-455 | A-456 |
| ABX05062 | Q-431 | K-432 | I-445 | L-451 | S-453 | L-454 | P-455 |
| ADL48215 | Q-425 | R-426 | L-439 | — | — | — | — |
| ACU70272 | Q-430 | T-431 | R-444 | A-450 | E-452 | G-453 | — |
| ADG88606 | Q-451 | R-452 | L-465 | L-471 | R-473 | D-474 | A-475 |
| AEW47954 | Q-426 | R-427 | V-440 | V-446 | — | — | — |
| ACD20223 | Q-447 | K-448 | F-461 | A-467 | T-469 | E-470 | — |
| AFC28171 | Q-425 | I-426 | L-439 | — | — | A-444 | L-445 |
| ADW03239 | Q-446 | L-447 | T-460 | N-466 | R-468 | T-469 | D-470 |
| CBT74727 | Q-450 | V-451 | L-464 | T-470 | A-472 | — | — |
| CAM04686 | Q-439 | Q-440 | I-453 | — | V-459 | P-460 | A-461 |
| ACZ20966 | F-451 | E-452 | L-465 | L-471 | A-473 | V-474 | D-475 |
| AEV38153 | Q-441 | K-442 | V-455 | G-461 | L-463 | Q-464 | K-465 |
| BAJ26623 | Q-421 | R-422 | L-435 | A-441 | S-443 | A-444 | A-445 |
| BAL91665 | Q-419 | M-420 | R-433 | — | — | — | — |
| ACV09397 | L-449 | E-450 | V-463 | L-469 | G-471 | V-472 | T-473 |
| ADG20157 | Q-420 | E-421 | V-434 | G-440 | A-442 | A-443 | V-444 |
| ADC61565 | R-423 | T-424 | V-437 | A-443 | V-445 | R-446 | P-447 |
| ACM66669 | Q-450 | V-451 | L-464 | T-470 | A-472 | — | — |
| ABD68843 | Q-425 | E-426 | F-439 | R-445 | G-447 | M-448 | — |
| ABS61373 | Q-421 | K-422 | F-435 | — | — | — | — |
| CBA30283 | Q-430 | Q-431 | F-444 | K-450 | — | — | — |
| AAA22266 | L-428 | V-429 | V-442 | L-448 | L-450 | — | — |
| CAA42814 | L-428 | E-429 | V-442 | F-448 | — | — | — |
| ABN51453 | L-451 | E-452 | V-465 | F-471 | — | — | — |
| ACZ00292 | Q-425 | R-426 | H-439 | R-445 | S-447 | — | — |
| AEI12946 | L-453 | E-454 | L-467 | I-473 | P-475 | I-476 | E-477 |
| ABS15474 | Q-441 | E-442 | I-455 | N-461 | E-463 | T-464 | R-465 |
| ADL51094 | Q-425 | E-426 | L-439 | K-445 | — | — | — |
| AFG34202 | Q-421 | K-422 | F-435 | — | — | — | — |
| AAN60220 | Q-421 | K-422 | F-435 | — | — | — | — |

### SPECIFIC EMBODIMENTS AND INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s) described herein.

What is claimed is:

1. A polypeptide comprising the amino acid sequence of a variant β-glucosidase, said variant β-glucosidase comprising one or more substitutions as compared to a reference β-glucosidase polypeptide, said one or more substitutions being selected from:
(a) a substitution at the amino acid position corresponding to I63 of SEQ ID NO:379 (an “I63 substitution”);
(b) a substitution at the amino acid position corresponding to A68 of SEQ ID NO:379 (an “A68 substitution”);
(c) a substitution at the amino acid position corresponding to A73 of SEQ ID NO:379 (an “A73 substitution”);
(d) a substitution at the amino acid position corresponding to Y74 of SEQ ID NO:379 (a “Y74 substitution”);
(e) a substitution at the amino acid position corresponding to V167 of SEQ ID NO:379 (a “V167 substitution”);
(f) a substitution at the amino acid position corresponding to V203 of SEQ ID NO:379 (a “V203 substitution”);
(g) a substitution at the amino acid position corresponding to I216 of SEQ ID NO:379 (a “I216 substitution”);
(h) a substitution at the amino acid position corresponding to T219 of SEQ ID NO:379 (a “T219 substitution”);
(i) a substitution at the amino acid position corresponding to K231 of SEQ ID NO:379 (a “K231 substitution”);
(j) a substitution at the amino acid position corresponding to M246 of SEQ ID NO:379 (a “M246 substitution”);
(k) a substitution at the amino acid position corresponding to F292 of SEQ ID NO:379 (a “F292 substitution”);
(l) a substitution at the amino acid position corresponding to S296 of SEQ ID NO:379 (a “S296 substitution”);
(m) a substitution at the amino acid position corresponding to M325 of SEQ ID NO:379 (an “M325 substitution”);
(n) a substitution at the amino acid position corresponding to N326 of SEQ ID NO:379 (an “N326 substitution”);
(o) a substitution at the amino acid position corresponding to E365 of SEQ ID NO:379 (an “E365 substitution”);
(p) a substitution at the amino acid position corresponding to Q366 of SEQ ID NO:379 (a “Q366 substitution”);
(q) a substitution at the amino acid position corresponding to Y399 SEQ ID NO:379 (a “Y399 substitution”);
(r) a substitution at the amino acid position corresponding to V400 SEQ ID NO:379 (a “V400 substitution”);
(s) a substitution at the amino acid position corresponding to W401 SEQ ID NO:379 (a “W401 substitution”);
(t) a substitution at the amino acid position corresponding to R410 SEQ ID NO:379 (an “R410 substitution”);
(u) a substitution at the amino acid position corresponding to D414 SEQ ID NO:379 (a “D414 substitution”);
(v) a substitution at the amino acid position corresponding to L427 SEQ ID NO:379 (an “L427 substitution”);
(w) a substitution at the amino acid position corresponding to T441 SEQ ID NO:379 (a “T441 substitution”);
(x) a substitution at the amino acid position corresponding to E450 SEQ ID NO:379 (an “E450 substitution”); and
wherein the one or more substitutions increases thermotolerance as compared to the reference β-glucosidase polypeptide.

2. The polypeptide of claim 1, wherein each of the one or more substitutions is selected from:
(a) an A73 substitution selected from A73G and A73S;
(b) a Y74 substitution that is Y74L;
(c) a V167 substitution that is V167A;
(d) a T219 substitution selected from T219A and T219S;
(e) a K231 substitution that is K231E;
(f) an M246 substitution selected from M246H and M246K;
(g) an F292 substitution selected from F292I and F292V;
(h) an S296 substitution that is S296T;
(i) an M325 substitution that is M325T;
(j) an N326 substitution that is N326G;
(k) a Y399 substitution that is Y399F;
(l) a W401 substitution that is W401F;
(m) a T441 substitution that is T441V; and
(n) an A449 substitution that is A449C.

3.-7. (canceled)

8. The polypeptide of claim 1, which further comprises one or more, two more, three or more, four or more, five or more, six or more, seven or more, or eight substitutions selected from:
(a) a D7H substitution;
(b) a D154N substitution;
(c) an I216V substitution;
(d) a D243H substitution;
(e) a D302R substitution;
(f) an S317H substitution;
(g) an E365G substitution; and
(h) a V400Y substitution.

9.-18. (canceled)

19. The polypeptide of claim 1, which comprises an amino acid sequence having at least 45%, at least 48%, at least 50%, at least 60%, at least 70%, at least 80%, at least 90%, at least 95%, or at least 97% sequence identity to SEQ ID NOs: 199-379.

20.-21. (canceled)

22. A composition comprising a polypeptide of claim 19.

23.-25. (canceled)

26. The composition of claim 22, wherein the polypeptide is produced by a host cell that recombinantly expresses said polypeptide.

27. A fermentation broth comprising a polypeptide according to claim 19.

28. The fermentation broth of claim 27, which is a filamentous fungal fermentation broth or cell-free fermentation broth.

29. (canceled)

30. A method for saccharifying biomass, comprising: treating biomass with the composition or with the fermentation broth of claim 27.

31. The method of claim 30, further comprising recovering fermentable sugars.

32. The method of claim 31, wherein the fermentable sugars comprise disaccharides, monosaccharides or a combination thereof.

33. (canceled)

34. The method of claim 32, wherein monosaccharides are produced by a β-glucosidase in said composition or said fermentation broth.

35. A method for producing a fermentation product, comprising:
(a) treating biomass with a composition or with a fermentation broth of claim 27, thereby producing fermentable sugars; and
(b) culturing a fermenting microorganism in the presence of the fermentable sugars produced in step (a) under fermentation conditions, thereby producing a fermentation product.

36. The method of claim 35, wherein said fermentable sugars comprise disaccharides or monosaccharides or a combination thereof.

37.-38. (canceled)

39. The method of claim 35, wherein the fermentation product is ethanol.

40. The method of claim 35, further comprising, prior to step (a), pretreating the biomass.

41. (canceled)

42. The method of claim 35, wherein said fermenting microorganism is selected from Zymonionas mobilis, Escherichia coli and Klebsiella oxytoca, Saccharomyces cerevisiae, Saccharomyces uvarum, Kluyveromyces fragilis, Kluyveromyces lactis, Candida pseudotropicalis, Trichoderma sp. and Pachysolen tannophilus.

43. (canceled)

44. The method of claim 35, wherein said biomass is corn stover, bagasses, sorghum, giant reed, elephant grass, miscanthus, Japanese cedar, wheat straw, switchgrass, hardwood pulp, softwood pulp, crushed sugar cane, energy cane, or Napier grass.

45. A nucleic acid comprising a nucleotide sequence encoding the polypeptide of claim 1.

46. The nucleic acid of claim 45 that has at least 50%, at least 60%, at least 70%, at least 80%, at least 90%, at least 95%, or at least 97% sequence identity to the nucleotide sequence of SEQ ID NOS: 1-198 or SEQ ID NO:381.

47-71. (canceled)
